(19) United States
(12) Patent Application Publication
(10) Pub. No.: US 2008/0274948 A1
(43) Pub. Date: Nov. 6, 2008

(54) Conjugates of Hydroxyalkyl Starch and G-Csf

(75) Inventors: Wolfram Eichner, Butzbach (DE); Helmut Knoller, Friedberg (DE); Katherina Lutterbeck, Friedberg (DE); Norbert Zander, Meine (DE); Ronald Frank, Meine-Grassel (DE); Harald Conradt, Braunschweig (DE); Klaus Sommermeyer, Rosbach (DE); Eckard Grabenhorst, Braunschweig (DE)

Correspondence Address: FISH & RICHARDSON P.C., PO BOX 1022, MINNEAPOLIS, MN, 55440-1022, US

(73) Assignee: FRESENIUS KABI DEUTSCHLAND GMBH, Bad Homburg v.d.H (DE)

(21) Appl. No.: 10/567,266

(22) PCT Filed: Aug. 6, 2004
(86) PCT No.: PCT/EP2004/008818

§ 371 Date: May 29, 2008

Related U.S. Application Data
(60) Provisional application No. 60/552,281, filed on Mar. 11, 2004.

(30) Foreign Application Priority Data
Aug. 8, 2003 (EP) PCT/EP03/08829
Aug. 8, 2003 (EP) PCT/EP03/08858
Aug. 8, 2003 (EP) PCT/EP03/08859
Mar. 11, 2004 (EP) 04005874.5

(51) Int. Cl. A61K 47/48; C07K 14/53; A61P 37/00

(52) U.S. Cl. 514/8; 530/397

(57) ABSTRACT

The present invention relates to conjugates of hydroxyalkyl starch and a granulocyte colony stimulating factor protein (G-CSF) wherein these conjugates are formed by a covalent linkage between the hydroxyalkyl starch or a derivative of the hydroxyalkyl starch and the protein. The present invention also relates to the methods of producing these conjugates and the use of these conjugates.

The present invention relates to conjugates of hydroxyalkyl starch and a granulocyte colony stimulating factor protein (G-CSF) wherein these conjugates are formed by a covalent linkage between the hydroxyalkyl starch or a derivative of the hydroxyalkyl starch and the protein. The present invention also relates to the method of producing these conjugates and the use of these conjugates.

It is generally accepted that the stability of proteins can be improved and the immune response against these proteins is reduced when these proteins are coupled to polymeric molecules. WO 94/28024 discloses that physiologically active proteins modified with polyethylene glycol (PEG) exhibit reduced immunogenicity and antigenicity and circulate in the bloodstream considerably longer than unconjugated proteins, i.e. have a reduced clearance rate.

G-CSF is a 21 kDa glycoprotein stabilized by two intrachain disulfide bonds and containing a single O-linked carbohydrate moiety. Mature G-CSF has 174 amino acids. In the animal body, G-CSF is synthesized by bone marrow stromal cells, macrophages and fibroblasts. It main function is to be a growth and differentiation factor for neutrophils and their precursor cells. However, it is also known in the art that G-CSF activates mature neutrophils. In addition, it stimulates growth/differentiation of various other haemopoietic progenitor cells (in synergy with additional haemopoietic growth factors) and promotes proliferation and migration of endothelial cells. Clinically, G-CSF is administered for the treatment of deficiencies in neutrophil levels (caused, e.g. by aplastic anaemia, myelodysplasia, AIDS, or chemotherapy).

WO 02/09766 discloses, among others, biocompatible protein-polymer compounds which are produced by conjugation of biologically active protein with a biocompatible polymer derivative. The biocompatible polymers used are highly reactive branched polymers, and the resulting conjugates contain a long linker between polymer derivative and protein. As biocompatible polymers, polymers of formula (P—OCH₂CO—NH—CHR—CO—)ₙ-L-Qₖ-A are described, wherein P and Q are polymeric residues and k may be 1 or 0. For P and Q, polyethylene glycol, polypropylene glycol, polyoxyethylene, polytrimethylene glycol, polylactic acid and its derivatives, polyacrylic acid and its derivatives, polyamino acid, polyvinyl alcohol, polyurethane, polyphosphazene, poly(L-lysine), polyalkylene oxide, polyacryl amide and water soluble polymers such as dextran or polysaccharide are mentioned. As proteins, among others, alpha, beta and gamma interferons, blood factors, cytokines such as interleukins, G-CSF, GM-CSF are mentioned. In the examples of WO 02/09766, only mono-, di- and tri-polyethyleneglycol derivatives are disclosed which are coupled exclusively to interferon and epidermal growth factor, and human growth hormone.

WO 94/01483 discloses biocompatible polymer conjugates which are formed by covalently binding a biologically inactive polymer or polymer derivative to a pharmaceutically pure, synthetic hydrophilic polymer via specific types of chemical bonds. As naturally occurring polymers and derivatives thereof, polysaccharides such as hyaluronic acid, proteoglycans such as chondroitin sulfates A, B and C, chitin, heparin, heparin sulfate, dextrans such as cyclodextran, hydroxyethyl cellulose, cellulose ether and starch, lipids such as triglycerides and phospholipids are disclosed. As synthetic polymers, among others, polyethylene and derivatives thereof are described having an average molecular weight of from about 100 to about 100,000. As proteins linked to the polymer or the polymer derivative, cytokines and growth factors are described, including interferons, tumor necrosis factors, interleukins, colony stimulating factors, growth factors such as osteogenic factor extract, epidermal growth factor, transforming growth factor, platelet derived growth factor, acidic fibroblast growth factor and others are disclosed. In all working examples of WO 94/01483, polyethylene glycols derivatives are used as polymer.

WO 96/11953 discloses N-terminally chemically modified protein compounds and methods of their production. Specifically, G-CSF compositions are described which result from coupling a water soluble polymer to the N terminus of G-CSF. In the context of WO 96/11953, also consensus interferone N-terminally coupled to water soluble polymers are disclosed. While a wide variety of water polymers are listed in WO 96/11953 (e.g. copolymers of ethylene glycol and propylene glycol, carboxymethyl cellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), poly(n-vinyl pyrrolidone)polyethylene glycol, polypropylene glycol homopolymers, polypropylene oxide/ethylene oxide copolymers or polyoxyethylated polyols), only PEGylated G-CSF or consensus IFN compositions are described in the working examples of WO 96/11953.

U.S. Pat. No. 6,555,660 B2 discloses polypeptide conjugates comprising a polypeptide exhibiting G-CSF activity and having an amino acid sequence that differs from the amino acid sequence of human G-CSF in at least one specified introduced and/or removed amino acid residue, wherein the conjugate comprises an attachment group for a non-polypeptide moiety, and further comprises at least one non-polypeptide moiety attached to the attachment group of the polypeptide. The non-polypeptide moiety may be a polymer such as polyethylene glycol or an oligosaccharide. In U.S. Pat. No. 6,555,660 B2, it is explicitly and unambiguously stated that PEG is by far the most preferred polymer molecule since it has only few reactive groups capable of cross-linking compared to polysaccharides such as dextran.

WO 97/30148 relates to polypeptide conjugates with reduced allergenicity comprising a polymeric carrier molecule having two or more polypeptide molecules coupled thereto. These conjugates are preferably part of compositions used in the personal care market. Said conjugates are produced by activating a polymeric carrier molecule, reacting two or more polypeptide molecules with the activated polymeric carrier molecule and blocking of residual active groups on the conjugate. As polymeric carrier molecule, a vast variety is listed in WO 97/30148, including such different groups of compound like natural or synthetic homopolymers such as polyols, polyamines, polycarboxylic acids and heteropolymers comprising at least two different attachment groups. Examples are given, which comprise star PEGs, branched PEGs, polyvinyl alcohols, polycarboxylates, polyvinylpyrrolidones and poly-D,L-amino acids. Among others, also dextrans such as carboxymethyl dextran, celluloses such as hydroxyethyl cellulose or hydroxypropyl cellulose, hydrolysates of chitosan, starches such as hydroxyethyl starches or hydroxypropyl starches, glycogen, agarose, guar gum, inulin, pullulan, xanthan gum, carrageenin, pectin, alginic acid etc. are disclosed. As polypeptides, only some enzymes are explicitly disclosed.

Baldwin, J. E. et al., Tetrahedron, vol. 27 (1981), pp. 1723-1726 describe the chemical modification of dextran and hydroxyethyl starch to give aldehyde substituted polymers which are allowed to react with hemoglobin to give soluble polymer-bound hemoglobins. These were shown to be capable of binding oxygen, but heart perfusion experiments clearly indicated that the polymer-bound hemoglobins were not suitable for use as blood substitutes.

WO 99/49897 describes conjugates of hemoglobin formed by reacting polysaccharides such as dextrane or hydroxyethyl starch with amino groups of the hemoglobin. As functional groups of the polysaccharide, aldehyde groups produced by oxidative saccharide ring-opening are used. As preferred reducing agent used, borane dimethylamine is disclosed. Moreover, WO 99/49897 is exclusively limited to hemoglobin.

WO 03/074087 relates to a method of coupling proteins to a starch-derived modified polysaccharide. The binding action between the protein and the polysaccharide, hydroxyalkyl starch, is a covalent linkage which is formed between the terminal aldehyde group or a functional group resulting from chemical modification of said terminal aldehyde group of the hydroxy alkyl starch molecule, and a functional group of the protein. As reactive group of the protein, amino groups, thio groups and carboxyl groups are disclosed, and aldehyde groups of the protein are not mentioned. Moreover, while a vast variety of possibilities of different linkages is given in the form of many lists, including different functional groups, theoretically suitable different linker molecules, and different chemical procedures, the working examples describe only two alternatives: first, an oxidized hydroxyethyl starch is used and coupled directly to proteins using ethyldimethylaminopropyl carbodiimide (EDC) activation, or a non-oxidized hydroxyethyl starch is used and coupled directly to a protein forming a Schiff's base which is subsequently reduced to the respective amine. Thus, the working examples of WO 03/074087 neither disclose a single conjugate coupled via a thio group or a carboxy group of the protein, nor describe a conjugate comprising hydroxyethyl starch, the protein, and one or more linker molecules. Additionally, no G-CSF molecule is used in the working examples.

Therefore, it was an object of the present invention to provide conjugates of hydroxyalkyl starch, preferably hydroxy ethyl starch, and G-CSF which are not yet described in the prior art.

It is a further object of the present invention to provide methods of producing these conjugates.

Therefore, the present invention relates to a method for preparing a conjugate comprising a protein and a polymer or a derivative thereof, wherein the polymer is a hydroxyalkyl starch (HAS) and the protein is a granulocyte colony stimulating factor (G-CSF), the method comprising reacting at least one functional group A of the polymer or the derivative thereof with at least one functional group Z of the protein and thereby forming a covalent linkage, wherein Z is selected from the group consisting of an amino group, a thiol group, an aldehyde group and a keto group, and
wherein, in case Z is an aldehyde group or a keto group, A comprises an amino group forming said linkage with Z, or
wherein, in case Z is an amino group, A is selected from the group consisting of a reactive carboxy group and an aldehyde group, a keto group or a hemiacetal group,
   wherein, in case A is an aldehyde group, a keto group or a hemiacetal group, the method further comprises introducing A in the polymer to give a polymer derivative
      by reacting the polymer with an at least bifunctional compound, one functional group of which reacts with the polymer and at least one other functional group of which is an aldehyde group, a keto group or a hemiacetal group, or is a functional group which is further chemically modified to give an aldehyde group, a keto group or a hemiacetal group, or
      by oxidizing the polymer to give at least one, in particular at least two aldehyde groups, or
   wherein, in case A is a reactive carboxy group, the method further comprises introducing A in the polymer to give a polymer derivative
      by selectively oxidizing the polymer at its reducing end and activating the resulting carboxy group, or
      by reacting the polymer at its non-oxidized reducing end with a carbonic diester, or
wherein, in case Z is a thiol group, A comprises
   a maleimido group or
   a halogenacetyl group
forming said linkage with Z.

Accordingly, the present invention also relates to a conjugate as obtainable by a method as described above.

The G-CSF can be produced by chemical synthetic procedures or can be of any human (see e.g. Burgess, A. W. et al. 1977, Stimulation by human placental conditioned medium of hemopoietic colony formation by human marrow cells, Blood 49 (1977), 573-583; Shah, R. G. et al. 1977, Characterization of colony-stimulating activity produced by human monocytes and phytohemagglutinin-stimulated lymphocytes, Blood 50 (1977), 811) or another mammalian source and can be obtained by purification from naturally occurring sources like human placenta, human blood or human urine. In addition, a lot of Epithelial carcinomas, acute myeloid leukemia cells and various tumor cell lines (bladder carcinomas, medulloblastomas), are capable to express this factor.

Furthermore, the expression G-CSF encompasses also a G-CSF variant wherein one or more amino acids (e.g. 1 to 25, preferably 1 to 10, more preferably 1 to 5, most preferred 1 or 2) have been exchanged by another amino acid and which exhibits G-CSF activity (see e.g. Riedhaar-Olson, J. F. et al. 1996, Identification of residues critical to the activity of human granulocyte colony-stimulating factor, Biochemistry 35:9034-9041 1996; U.S. Pat. Nos. 5,581,476; 5,214,132; 5,362,853; 4,904,584). The measurement of G-CSF activity is described in the art (for measurement of G-CSF activity in vitro see e.g. Shirafuji, N. et al. 1989, A new bioassay for human granulocyte colony-stimulating factor (hG-CSF) using murine myeloblastic NFS-60 cells as targets and estimation of its levels in sera from normal healthy persons and patients with infectious and hematological disorders, Exp. Hematol. 1989, 17, 116-119; for measurement of G-CSF activity in vivo see e.g. Tanaka, H. et al. 1991, Pharmacokinetics of recombinant human granulocyte colony-stimulating factor conjugated to polyethylene glycol in rats, Cancer Research 51, 3710-3714, 1991). Further publications where tests for the measurement of the activity of G-CSF are U.S. Pat. No. 6,555,660; Nohynek, G. J. et al. 1997, Comparison of the potency of glycosylated and nonglycosylated recombinant human granulocyte colony-stimulating factors in neutropenic and nonneutropenic CD rats, Cancer Chemother Pharmacol (1997) 39; 259-266.

Preferably, the G-CSF is recombinantly produced. This includes prokaryotic or eukaryotic host expression of exogenous DNA sequences obtained by genomic or cDNA cloning or by DNA synthesis. Suitable prokaryotic hosts include various bacteria such as E. coli. Suitable eukaryotic hosts include yeast such as S. cerevisiae and mammalian cells such as Chinese hamster ovary cells and monkey cells.

The recombinant production of a protein is known in the art. In general, this includes the transfection of host cells with an appropriate expression vector, the cultivation of the host cells under conditions which enable the production of the protein and the purification of the protein from the host cells. For detailed information see e.g. Souza, L. M. et al. 1986, Recombinant human granulocyte colony-stimulating factor: effects on normal and leukemic myeloid cells, Science 1986 232:61-65, 1986; Nagata, S. et. al. 1986, Molecular cloning and expression of cDNA for human granulocyte colony-stimulating factor, Nature 319:415-418, 1986; Komatsu, Y. et al. 1987, Cloning of granulocyte colony-stimulating factor cDNA from human macrophages and its expression in Escherichia coli, Jpn J Cancer Res. 1987 78 (11):1179-1181.

In a preferred embodiment, the G-CSF has the amino acid sequence of human mature G-CSF (see e.g.; Nagata, S. et. al. 1986, Molecular cloning and expression of cDNA for human granulocyte colony-stimulating factor, Nature 319:415-418, 1986), and may further contain a methionin at its amino terminus, which then results in a protein of 175 amino acids. Furthermore, instead of the methionine, G-CSF may contain a serine or a threonine residue.

The G-CSF used in the methods of the present invention and the conjugates according to the present invention may comprise one carbohydrate side chain attached to the G-CSF via O-linked glycosylation at the position Thr 133, i.e. the G-CSF is glycosylated (V. Gervais et al., Eur. J. Biochem. 1997, 247, 386-395). The structure of the carbohydrate side chain may be NeuNAc(alpha2-3)Gal(beta1-3)[NeuNAc(alpha2-6)]GalNAc and (alpha2-3)Gal(beta1-3)GalNAc (NeuNAc=N-acetylneuramic acid, GalNAc=N-acetylgalactosamine).

Modification of G-CSF and other polypeptides so as to introduce at least one additional carbohydrate chain as compared to the native polypeptide has been suggested (U.S. Pat. No. 5,218,092). Depending on the host employed, the G-CSF expression product may be glycosylated with mammalian or other eukaryotic carbohydrates. Usually, when G-CSF is produced in eukaryotic cells, the protein is posttranslationally glycosylated. Consequently, the carbohydrate side chain may have been attached to the G-CSF during biosynthesis in mammalian, especially human, insect or yeast cells.

Recombinant human G-CSF (rhG-CSF) is generally used for treating various forms of leukopenia. Thus, commercial preparations of rhG-CSF are available under the names filgrastim (Gran® and Neupogen®), lenograstim (Neutrogin® and Granocyte®) and nartograstim (Neu-up®). Grand and Neupogen® are non-glycosylated and produced in recombinant E. coli cells. Neutrogin® and Granocyte® are glycosylated and produced in recombinant CHO cells and Neu-up® is non-glycosylated with five amino acids substituted at the N-terminal region of intact rhG-CSF produced in recombinant E. coli cells.

As glycosylated protein, any glycosylated G-CSF such as Granocyte® may be employed. As non-glycosylated G-CSF, any non-glycosylated G-CSF such as Neupogen® may be employed in the methods and conjugate according to the present invention.

Furthermore, at position −1, G-CSF may contain a methionine amino acid residue, a serine residue, or a threonine residue.

In the context of the present invention, the term “hydroxyalkyl starch” (HAS) refers to a starch derivative which has been substituted by at least one hydroxyalkyl group. A preferred hydroxyalkyl starch of the present invention has a constitution according to formula (I)

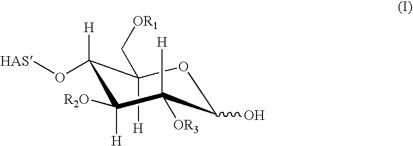

wherein the reducing end of the starch molecule is shown in the non-oxidized form and the terminal saccharide unit is shown in the acetal form which, depending on e.g. the solvent, may be in equilibrium with the aldehyde form.

The term hydroxyalkyl starch as used in the present invention is not limited to compounds where the terminal carbohydrate moiety comprises hydroxyalkyl groups R₁, R₂, and/or R₃ as depicted, for the sake of brevity, in formula (I), but also refers to compounds in which at least one hydroxy group present anywhere, either in the terminal carbohydrate moiety and/or in the remaining part of the starch molecule, HAS′, is substituted by a hydroxyalkyl group R₁, R₂, or R₃.

Hydroxyalkyl starch comprising two or more different hydroxyalkyl groups are also possible.

The at least one hydroxyalkyl group comprised in HAS may contain two or more hydroxy groups. According to a preferred embodiment, the at least one hydroxyalkyl group comprised in HAS contains one hydroxy group.

The expression “hydroxyalkyl starch” also includes derivatives wherein the alkyl group is mono- or polysubstituted. In this context, it is preferred that the alkyl group is substituted with a halogen, especially fluorine, or with an aryl group. Furthermore, the hydroxy group of a hydroxyalkyl group may be esterified or etherified.

Furthermore, instead of alkyl, also linear or branched substituted or unsubstituted alkene groups may be used.

Hydroxyalkyl starch is an ether derivative of starch. Besides of said ether derivatives, also other starch derivatives can be used in the context of the present invention. For example, derivatives are useful which comprise esterified hydroxy groups. These derivatives may be e.g. derivatives of unsubstituted mono- or dicarboxylic acids with 2-12 carbon atoms or of substituted derivatives thereof. Especially useful are derivatives of unsubstituted monocarboxylic acids with 2-6 carbon atoms, especially derivatives of acetic acid. In this context, acetyl starch, butyl starch and propyl starch are preferred.

Furthermore, derivatives of unsubstituted dicarboxylic acids with 2-6 carbon atoms are preferred.

In the case of derivatives of dicarboxylic acids, it is useful that the second carboxy group of the dicarboxylic acid is also esterified. Furthermore, derivatives of monoalkyl esters of dicarboxylic acids are also suitable in the context of the present invention.

For the substituted mono- or dicarboxylic acids, the substitute groups may be preferably the same as mentioned above for substituted alkyl residues.

Techniques for the esterification of starch are known in the art (see e.g. Klemm D. et al, Comprehensive Cellulose Chemistry Vol. 2, 1998, Whiley-VCH, Weinheim, N.Y., especially chapter 4.4, Esterification of Cellulose (ISBN 3-527-29489-9).

According to a preferred embodiment of the present invention, hydroxyalkyl starch according to formula (I) is employed. In formula (I), the saccharide ring described explicitly and the residue denoted as HAS′ together represent the preferred hydroxyalkyl starch molecule. The other saccharide ring structures comprised in HAS′ may be the same as or different from the explicitly described saccharide ring.

As far as the residues R₁, R₂ and R₃ according to formula (I) are concerned there are no specific limitations. According to a preferred embodiment, R₁, R₂ and R₃ are independently hydrogen or a hydroxyalkyl group, a hydroxyaryl group, a hydroxyaralkyl group or a hydroxyalkaryl group having of from 2 to 10 carbon atoms in the respective alkyl residue. Hydrogen and hydroxyalkyl groups having of from 2 to 10 are preferred. More preferably, the hydroxyalkyl group has from 2 to 6 carbon atoms, more preferably from 2 to 4 carbon atoms, and even more preferably from 2 to 4 carbon atoms. “Hydroxyalkyl starch” therefore preferably comprises hydroxyethyl starch, hydroxypropyl starch and hydroxybutyl starch, wherein hydroxyethyl starch and hydroxypropyl starch are particularly preferred and hydroxyethyl starch is most preferred.

The alkyl, aryl, aralkyl and/or alkaryl group may be linear or branched and suitably substituted.

Therefore, the present invention also relates to a method as described above wherein R₁, R₂ and R₃ are independently hydrogen or a linear or branched hydroxyalkyl group with from 1 to 6 carbon atoms.

Thus, R₁, R₂ and R₃ preferably may be hydroxyhexyl, hydroxypentyl, hydroxybutyl, hydroxypropyl such as 2-hydroxypropyl, 3-hydroxypropyl, 2-hydroxyisopropyl, hydroxyethyl such as 2-hydroxyethyl, hydrogen and the 2-hydroxyethyl group being especially preferred.

Therefore, the present invention also relates to a method and a conjugate as described above wherein R₁, R₂ and R₃ are independently hydrogen or a 2-hydroxyethyl group, an embodiment wherein at least one residue R₁, R₂ and R₃ being 2-hydroxyethyl being especially preferred.

Hydroxyethyl starch (HES) is most preferred for all embodiments of the present invention.

Therefore, the present invention relates to the method and the conjugate as described above, wherein the polymer is hydroxyethyl starch and the polymer derivative is a hydroxyethyl starch derivative.

Hydroxyethyl starch (HES) is a derivative of naturally occurring amylopectin and is degraded by alpha-amylase in the body. HES is a substituted derivative of the carbohydrate polymer amylopectin, which is present in corn starch at a concentration of up to 95% by weight. HES exhibits advantageous biological properties and is used as a blood volume replacement agent and in hemodilution therapy in the clinics (Sommermeyer et al., 1987, Krankenhauspharmazie, 8 (8), 271-278; and Weidler et al., 1991, Arzneim.-Forschung/Drug Res., 41, 494-498).

Amylopectin consists of glucose moieties, wherein in the main chain alpha-1,4-glycosidic bonds are present and at the branching sites alpha-1,6-glycosidic bonds are found. The physical-chemical properties of this molecule are mainly determined by the type of glycosidic bonds. Due to the nicked alpha-1,4-glycosidic bond, helical structures with about six glucose-monomers per turn are produced. The physico-chemical as well as the biochemical properties of the polymer can be modified via substitution. The introduction of a hydroxyethyl group can be achieved via alkaline hydroxyethylation. By adapting the reaction conditions it is possible to exploit the different reactivity of the respective hydroxy group in the unsubstituted glucose monomer with respect to a hydroxyethylation. Owing to this fact, the skilled person is able to influence the substitution pattern to a limited extent.

HES is mainly characterized by the molecular weight distribution and the degree of substitution. There are two possibilities of describing the substitution degree:
1. The degree can be described relatively to the portion of substituted glucose monomers with respect to all glucose moieties.
2. The degree of substitution can be described as the molar substitution, wherein the number of hydroxyethyl groups per glucose moiety are described.

In the context of the present invention, the degree of substitution, denoted as DS, relates to the molar substitution, as described above.

HES solutions are present as polydisperse compositions, wherein each molecule differs from the other with respect to the polymerisation degree, the number and pattern of branching sites, and the substitution pattern. HES is therefore a mixture of compounds with different molecular weight. Consequently, a particular HES solution is determined by average molecular weight with the help of statistical means. In this context, Mₙ is calculated as the arithmetic mean depending on the number of molecules. Alternatively, M_{w} (or MW), the weight mean, represents a unit which depends on the mass of the HES.

In the context of the present invention, hydroxyethyl starch may preferably have a mean molecular weight (weight mean) of from 1 to 300 kD. Hydroxyethyl starch can further exhibit a preferred molar degree of substitution of from 0.1 to 0.8 and a preferred ratio between C₂:C₆ substitution in the range of from 2 to 20 with respect to the hydroxyethyl groups.

The term “mean molecular weight” as used in the context of the present invention relates to the weight as determined according to Sommermeyer et al., 1987, Krankenhauspharmazie, 8 (8), 271-278; and Weidler et al., 1991, Arzneim.-Forschung/Drug Res., 41, 494-498.

According to a preferred embodiment of the present invention, the mean molecular weight of hydroxyethyl starch employed is from 1 to 300 kD, more preferably from 2 to 200 kD, more preferably of from 4 to 130 kD, more preferably of from 4 to 70 kD.

An example for HES with a mean molecular weight of about 130 kD is Voluven® from Fresenius. Voluven® is an artificial colloid, employed, e.g., for volume replacement used in the therapeutic indication for therapy and prophylaxis of hypovolaemia. The characteristics of Voluven® are a mean molecular weight of 130,000+/−20,000 D, a molar substitution of 0.4 and a C2:C6 ratio of about 9:1.

Therefore, the present invention also relates to a method and to conjugates as described above wherein the hydroxyalkyl starch is hydroxyethyl starch having a mean molecular weight of from 4 to 70 kD.

Preferred ranges of the mean molecular weight are, e.g., 4 to 70 kD or 10 to 70 kD or 12 to 70 kD or 18 to 70 kD or 50 to 70 kD or 4 to 50 kD or 10 to 50 kD or 12 to 50 kD or 18 to 50 kD or 4 to 18 kD or 10 to 18 kD or 12 to 18 kD or 4 to 12 kD or 10 to 12 kD or 4 to 10 kD.

According to particularly preferred embodiments of the present invention, the mean molecular weight of hydroxyethyl starch employed is in the range of from more than 4 kD and below 70 kD, such as about 10 kD, or in the range of from 9 to 10 kD or from 10 to 11 kD or from 9 to 11 kD, or about 12 kD, or in the range of from 11 to 12 kD or from 12 to 13 kD or from 11 to 13 kD, or about 18 kD, or in the range of from 17 to 18 kD or from 18 to 19 kD or from 17 to 19 kD, or about 50 kD, or in the range of from 49 to 50 kD or from 50 to 51 kD or from 49 to 51 kD.

As far as the degree of substitution (DS) is concerned, DS is preferably at least 0.1, more preferably at least 0.2, and more preferably at least 0.4. Preferred ranges of DS are from 0.1 to 0.8, more preferably from 0.2 to 0.8, more preferably from 0.3 to 0.8 and even more preferably from 0.4 to 0.8, still more preferably from 0.1 to 0.7, more preferably from 0.2 to 0.7, more preferably from 0.3 to 0.7 and more preferably from 0.4 to 0.7. Particularly preferred values of DS are, e.g., 0.1, 0.2, 0.3, 0.4, 0.5, 0.6, 0.7 or 0.8, with 0.2, 0.3, 0.4, 0.5, 0.6, 0.7 or 0.8 being more preferred, 0.3, 0.4, 0.5, 0.6, 0.7 or 0.8 being even more preferred, 0.4, 0.5, 0.6, 0.7 or 0.8 being still more preferred and, e.g. 0.4 and 0.7 being particularly preferred.

Particularly preferred combinations of molecular weight of the hydroxyalkyl starch, preferably hydroxyethyl starch, and its degree of substitution DS are, e.g., 10 kD and 0.4 or 10 kD aid 0.7 or 12 kD and 0.4 or 12 kD and 0.7 or 18 kD and 0.4 or 18 kD and 0.7 or 50 kD and 0.4 or 50 kD and 0.7.

In another preferred embodiment of the present invention, the hydroxyethyl starch (as employed as well as contained in the conjugates described herein) has a molecular weight of from about 20 kD to about 130 kD (i.e. about 40 kD, about 50 kD, about 60 kD, about 70 kD, about 80 kD, about 90 kD, about 100 kD, about 110 kD, about 120 kD, about 130 kD) preferably a mean molecular weight of about 30 kD to about 100 kD, more preferably from about 40 to about 70 kD and a degree of substitution from 0.4 to 0.8, more preferred from 0.5 to 0.8.

In this context the term “about 30 kD” is understood to relate to a mean molecular weight in the range of from 25 kD to 34 kD, i.e. including also starches having a mean molecular weight of 26, 27, 28, 29, 31, 32, 33 or 34 kD.

In this context the term “about 40 kD” is understood to relate to a mean molecular weight in the range of from 35 kD to 44 kD, i.e. including also starches having a mean molecular weight of 36, 37, 38, 39, 41, 42, 43 or 44 kD.

In this context the term “about 50 kD” is understood to relate to a mean molecular weight in the range of from 45 kD to 54 kD, i.e. including also starches having a mean molecular weight of 46, 47, 48, 49, 51, 52, 53 or 54 kD.

In this context the term “about 60 kD” is understood to relate to a mean molecular weight in the range of from 55 kD to 64 kD, i.e. including also starches having a mean molecular weight of 56, 57, 58, 59, 61, 62, 63 or 64 kD.

In this context the term “about 70 kD” is understood to relate to a mean molecular weight in the range of from 65 kD to 74 kD, i.e. including also starches having a mean molecular weight of 66, 67, 68, 69, 71, 72, 73 or 74 kD.

In this context the term “about 80 kD” is understood to relate to a mean molecular weight in the range of from 75 kD to 84 kD, i.e. including also starches having a mean molecular weight of 76, 77, 78, 79, 81, 82, 83 or 84 kD.

In this context the term “about 90 kD” is understood to relate to a mean molecular weight in the range of from 85 kD to 94 kD, i.e. including also starches having a mean molecular weight of 86, 87, 88, 89, 91, 92, 93 or 94 kD.

In this context the term “about 100 kD” is understood to relate to a mean molecular weight in the range of from 95 kD to 104 kD, i.e. including also starches having a mean molecular weight of 96, 97, 98, 99, 101, 102, 103 or 104 kD.

In this context the term “about 110 kD” is understood to relate to a mean molecular weight in the range of from 105 kD to 114 kD, i.e. including also starches having a mean molecular weight of 106, 107, 108, 109, 111, 112, 113 or 114 kD.

In this context the term “about 120 kD” is understood to relate to a mean molecular weight in the range of from 115 kD to 124 kD, i.e. including also starches having a mean molecular weight of 116, 117, 118, 119, 121, 122, 123 or 124 kD.

In this context the term “about 130 kD” is understood to relate to a mean molecular weight in the range of from 125 kD to 134 kD, i.e. including also starches having a mean molecular weight of 126, 127, 128, 129, 131, 132, 133 or 134 kD.

Accordingly the embodiment described above comprises a hydroxethyl starch (and the conjugates described herein comprising the hydroxylethyl starch as well as the methods described herein employing hydroxylethyl starch) that has a mean molecular weight of about 30 kD and a degree of substitution of 0.4 or 0.5 or 0.6 or 0.7 or 0.8, preferably 0.6, 0.7 or 0.8.

Accordingly the embodiment described above also comprises a hydroxethyl starch (and the conjugates described herein comprising the hydroxylethyl starch as well as the methods described herein employing hydroxyethyl starch) that has a mean molecular weight of about 40 kD and a degree of substitution of 0.4 or 0.5 or 0.6 or 0.7 or 0.8, preferably 0.6, 0.7 or 0.8.

Accordingly the embodiment described above also comprises a hydroxethyl starch (and the conjugates described herein comprising the hydroxylethyl starch as well as the methods described herein employing hydroxyethyl starch) that has a mean molecular weight of about 50 kD and a degree of substitution of 0.4 or 0.5 or 0.6 or 0.7 or 0.8, preferably 0.6, 0.7 or 0.8.

Accordingly the embodiment described above also comprises a hydroxethyl starch (and the conjugates described herein comprising the hydroxylethyl starch as well as the methods described herein employing hydroxyethyl starch) that has a mean molecular weight of about 60 kD and a degree of substitution of 0.4 or 0.5 or 0.6 or 0.7 or 0.8, preferably 0.6, 0.7 or 0.8.

Accordingly the embodiment described above also comprises a hydroxethyl starch (and the conjugates described herein comprising the hydroxylethyl starch as well as the methods described herein employing hydroxyethyl starch) that has a mean molecular weight of about 70 kD and a degree of substitution of 0.4 or 0.5 or 0.6 or 0.7 or 0.8, preferably 0.6, 0.7 or 0.8.

Accordingly the embodiment described above also comprises a hydroxethyl starch (and the conjugates described herein comprising the hydroxylethyl starch as well as the methods described herein employing hydroxyethyl starch) that has a mean molecular weight of about 80 kD and a degree of substitution of 0.4 or 0.5 or 0.6 or 0.7 or 0.8, preferably 0.6, 0.7 or 0.8.

Accordingly the embodiment described above also comprises a hydroxethyl starch (and the conjugates described herein comprising the hydroxylethyl starch as well as the methods described herein employing hydroxyethyl starch) that has a mean molecular weight of about 90 kD and a degree of substitution of 0.4 or 0.5 or 0.6 or 0.7 or 0.8, preferably 0.6, 0.7 or 0.8.

Accordingly the embodiment described above also comprises a hydroxethyl starch (and the conjugates described herein comprising the hydroxylethyl starch as well as the methods described herein employing hydroxyethyl starch) that has a mean molecular weight of about 100 kD and a degree of substitution of 0.4 or 0.5 or 0.6 or 0.7 or 0.8, preferably 0.6, 0.7 or 0.8.

Accordingly the embodiment described above also comprises a hydroxethyl starch (and the conjugates described herein comprising the hydroxylethyl starch as well as the methods described herein employing hydroxyethyl starch) that has a mean molecular weight of about 110 kD and a degree of substitution of 0.4 or 0.5 or 0.6 or 0.7 or 0.8, preferably 0.6, 0.7 or 0.8.

Accordingly the embodiment described above comprises a hydroxethyl starch (and the conjugates described herein comprising the hydroxylethyl starch as well as the methods described herein employing hydroxyethyl starch) that has a mean molecular weight of about 120 kD and a degree of substitution of 0.4 or 0.5 or 0.6 or 0.7 or 0.8, preferably 0.6, 0.7 or 0.8.

Accordingly the embodiment described above comprises a hydroxethyl starch (and the conjugates described herein comprising the hydroxylethyl starch as well as the methods described herein employing hydroxyethyl starch) that has a mean molecular weight of about 130 kD and a degree of substitution of 0.4 or 0.5 or 0.6 or 0.7 or 0.8, preferably 0.6, 0.7 or 0.8.

An example of HES having a mean molecular weight of about 130 kD is a HES with a degree of substitution of 0.2 to 0.8 such as 0.2, 0.3, 0.4, 0.5, 0.6, 0.7, or 0.8, preferably of 0.4 to 0.7 such as 0.4, 0.5, 0.6, or 0.7.

As far as the ratio of C₂:C₆ substitution is concerned, said substitution is preferably in the range of from 2 to 20, more preferably in the range of from 2 to 15 and even more preferably in the range of from 3 to 12.

According to a further embodiment of the present invention, also mixtures of hydroxyethyl starches may be employed having different mean molecular weights and/or different degrees of substitution and/or different ratios of C₂:C₆ substitution. Therefore, mixtures of hydroxyethyl starches may be employed having different mean molecular weights and different degrees of substitution and different ratios of C₂:C₆ substitution, or having different mean molecular weights and different degrees of substitution and the same or about the same ratio of C₂:C₆ substitution, or having different mean molecular weights and the same or about the same degree of substitution and different ratios of C₂:C₆ substitution, or having the same or about the same mean molecular weight and different degrees of substitution and different ratios of C₂:C₆ substitution, or having different mean molecular weights and the same or about the same degree of substitution and the same or about the same ratio of C₂:C₆ substitution, or having the same or about the same mean molecular weights and different degrees of substitution and the same or about the same ratio of C₂:C₆ substitution, or having the same or about the same mean molecular weight and the same or about the same degree of substitution and different ratios of C₂:C₆ substitution, or having about the same mean molecular weight and about the same degree of substitution and about the same ratio of C₂:C₆ substitution.

In different conjugates and/or different methods according to the present invention, different hydroxyalkyl starches, preferably different hydroxyethyl starches and/or different hydroxyalkyl starch mixtures, preferably different hydroxyethyl starch mixtures, may be employed.

According to one embodiment of the present invention, the functional group Z of the protein is an aldehyde group or a keto group. Therefore, the present invention relates to a method and conjugates as described above, wherein the functional group Z of the protein is an aldehyde group or a keto group.

While there are no general restrictions as to the location of the aldehyde or keto group within the protein, the aldehyde or keto group is, according to a preferred embodiment of the present invention, located in a carbohydrate side chain of the protein. Therefore, in the context of this embodiment, a glycosylated protein is employed.

As glycosylated protein, any glycosylated G-CSF such as Granocyte® may be employed.

In the context of the present invention, the term “carbohydrate side chain” refers to hydroxyaldehydes or hydroxyketones as well as to chemical modifications thereof (see Römpp Chemielexikon, Thieme Verlag Stuttgart, Germany, 9^{th} edition 1990, Volume 9, pages 2281-2285 and the literature cited therein). Furthermore, it also refers to derivatives of naturally occurring carbohydrate moieties like, galactose, N-acetylneuramic acid, and N-acetylgalactosamine) and the like. In case a mutant of G-CSF is employed being N-glycosylated, a carbohydrate moiety may be mannose.

In an even more preferred embodiment, the aldehyde group or the keto group is part of a galactose residue of the carbohydrate side chain. This galactose residue can be made available for reaction with the functional group A comprised in the polymer or polymer derivative by removal of terminal sialic acids, followed by oxidation, as described hereinunder.

In a still further preferred embodiment, the polymer or polymer derivative comprising functional group A is linked to a sialic acid residue of the carbohydrate side chains, preferably the terminal sialic acid residue of the carbohydrate side chain.

Oxidation of terminal carbohydrate moieties can be performed either chemically or enzymatically.

Methods for the chemical oxidation of carbohydrate moieties of polypeptides are known in the art and include the treatment with periodate (Chamow et al., 1992, J. Biol. Chem., 267, 15916-15922).

By chemically oxidizing, it is in principle possible to oxidize any carbohydrate moiety, being terminally positioned or not. However, by choosing mild reaction conditions it is possible to preferably oxidize the terminal sialic acid of a carbohydrate side chain to give the aldehyde group or the keto group.

According to one embodiment of the present invention, said mild reaction conditions relate to reacting the protein with a suitable aqueous periodate solution, having a preferred periodate concentration in the range of from 1 to 50 mM, more preferably of from 1 to 25 mM and especially preferably of from 1 to 10 mM such as about 1 mM, and at a preferred reaction temperature of from 0 to 40° C. and especially preferably of from 0 to 21° C. such as about 0° C., and for a preferred reaction time of from 5 min to 5 h, more preferably from 10 min to 2 h and especially preferably from 10 min. to 1 h such as about 1 h. The preferred molar ratio of periodate:protein is from 1:200 to 1:1 and more preferably from 1:50 to 1:5. such as about 15:1.

Therefore, the present invention also relates to a method and a conjugate as described above, wherein, prior to the reaction of the protein and the polymer or polymer derivate, a glycosylated protein is reacted with a periodate solution to give a protein having an aldehyde group or a keto group located in the oxidized carbohydrate side chain.

Alternatively, the carbohydrate side chain may be oxidized enzymatically. Enzymes for the oxidation of the individual carbohydrate side chain are known in the art, e.g. in the case of galactose the enzyme is galactose oxidase. If it is intended to oxidize terminal galactose moieties, it will be eventually necessary to remove terminal sialic acids (partially or completely) if the polypeptide has been produced in cells capable of attaching sialic acids to carbohydrate chains, e.g. in mammalian cells or in cells which have been genetically modified to be capable of attaching sialic acids to carbohydrate chains. Chemical or enzymatic methods for the removal of sialic acids are known in the art (Chaplin and Kennedy (eds.), 1996, Carbohydrate Analysis: a practical approach, especially Chapter 5 Montreuill, Glycoproteins, pages 175-177; IRL Press Practical approach series (ISBN 0-947946-44-3)).

According to another preferred embodiment of the present invention, the aldehyde group or keto group may be located at the N terminus of the protein and is accessible by suitable oxidation. Especially in the case that a hydroxy group containing amino acid is located at the N terminus of the protein, such as threonine or serine, oxidation of said N-terminal amino acid can be carried out leading to said keto group or an aldehyde group. Theronine, is the N terminal amino acid in human derived G-CSF. An additional N-terminal serine or threonine may be introduced in any protein showing G-CSF like activity by molecular biological methods. This protein or the protein expressing the human amino acid sequence may be produced by expression in prokaryotic or eukaryotic cells such as bacteria, mammalian, insect or yeast cells, and which may or may not be glycosylated. As method for the chemical oxidation of the suitable N-terminal amino acid, any conceivable method may be applied, with the oxidation with periodate being preferred.

According to a further preferred embodiment of the present invention, said mild reaction conditions relate to reacting the protein with a suitable aqueous periodate solution, having a preferred periodate concentration in the range of from 1 to 50 mM, more preferably of from 1 to 25 mM and especially preferably of from 1 to 10 mM such as about 1 mM, and at a preferred reaction temperature of from 0 to 40° C. and especially preferably of from 0 to 21° C. such as about 0° C.; and for a preferred reaction time of from 5 min to 5 h, more preferably from 10 min to 2 h and especially preferably from 10 min. to 1 h such as about 1 h. The preferred molar ratio of periodate:protein is from 1:200 to 1:1 and more preferably from 1:50 to 1:5. such as about 15:1.

Therefore, the present invention also relates to a method and a conjugate as described above, wherein the aldehyde group or the keto group is located in a carbohydrate side chain of the protein and/or at the N-terminal group of the protein.

The oligosaccharide pattern of proteins produced in eukaryotic cells thus having been posttranslationally glycosylated, are not identical to the human derived proteins. Moreover, many glycosylated proteins do not have the desired number of terminal sialic acid residues masking a further carbohydrate moiety such as a galactose residue. Those further carbohydrate moieties such as a galactose residue, however, if not masked, are possibly responsible for disadvantages such as a shorter plasma half-life of the protein in possible uses of the protein as a medicament. It was surprisingly found that by providing a protein conjugate formed by a hydroxyalkyl starch polymer, preferably a hydroxyethyl starch polymer, which is covalently linked, e.g. via an oxime linkage as disclosed hereinunder, to a carbohydrate moiety of a carbohydrate side chain of the protein, either directly or via at least one linker compounds such as one or two linker compounds, it is possible to overcome at least the above mentioned disadvantage. Hence it is believed that by coupling a hydroxyalkyl starch polymer or derivative thereof, preferably a hydroxyethyl starch polymer or a derivative thereof, to at least one carbohydrate side chain of a glycosylated protein, the lack of suitable terminal carbohydrate residues located at a carbohydrate side chain is compensated. According to another aspect of the invention, providing the respective conjugate with a hydroxyalkyl starch polymer or derivative thereof, preferably a hydroxyethyl starch polymer or a derivative thereof, coupled to the oxidized carbohydrate moiety as described above, does not only compensate the disadvantage but provides a protein conjugate having better characteristics in the desired field of use than the respective naturally occurring protein. Therefore, the respective conjugates according to the invention have a compensational and even a synergistic effect on the protein. It also possible that even proteins which are identical to human proteins or which are human proteins do not have the desired number of suitable masking terminal carbohydrate residues such as silaic acid residues at naturally occurring carbohydrate moieties. In such cases, providing the respective conjugate with a hydroxyalkyl starch polymer or derivative thereof, preferably a hydroxyethyl starch polymer or a derivative thereof, coupled to the oxidized carbohydrate moiety as described above, does not only overcome and compensate a disadvantage of an artificially produced protein, but improves the characteristics of the a natural naturally occurring protein. As to the functional group of the hydroxyalkyl starch, preferably hydroxyethyl starch, or a derivative thereof, which is coupled to the aldehyde group or keto group of the oxidized carbohydrate moiety of the protein, reference is made to the functional groups A as disclosed hereinunder. This general concept is not only applicable to glycosylated G-CSF, but principally to all glycosylated having said lack of terminal carbohydrate residues. Among others, erythropoietin (EPO), interferone beta 1a (IFN beta 1a), ATIII, factor VII, factor VIII, factor IX, alpha1-antitrypsin (A1AT), htPA, or GM-CSF may be mentioned.

Therefore, the present invention also relates to the use of hydroxyalkyl starch, preferably hydroxyethyl starch, or a derivative thereof, for compensating the lack of terminal carbohydrate residues, preferably sialic acid residues, in naturally occurring or posttranslationally attached carbohydrate moieties of a protein, by covalently coupling the starch or derivative thereof to at least one oxidized carbohydrate moiety of a protein having at least one keto or aldehyde group.

Accordingly, the present invention also relates to a method for compensating the lack of terminal carbohydrate residues, preferably sialic acid residues, in naturally occurring or posttranslationally attached carbohydrate moieties of a protein, by covalently coupling hydroxyalkyl starch, preferably hydroxyethyl starch, or a derivative thereof to at least one oxidized carbohydrate moiety of a protein having at least one keto or aldehyde group, preferably via an oxime linkage.

Moreover, the present invention also relates to a conjugate formed by covalent linkage of a hydroxyalkyl starch, preferably hydroxyethyl starch, or a derivative thereof, to at least one oxidized carbohydrate moiety of a protein, said protein being either isolated from natural sources or produced by expression in eukaryotic cells, such as mammalian, insect or yeast cells, said carbohydrate moiety having at least one keto or aldehyde group, wherein the conjugate has in the desired field of use, preferably the use as medicament, the same or better characteristics than the respective unmodified protein.

In case functional group Z of the protein is an aldehyde group or a keto group, functional group A of the polymer or the derivative thereof comprises an amino group according to the structure —NH—.

Therefore, the present invention also relates to a method and a conjugate as described above wherein the functional group A capable of being reacted with the optionally oxidized reducing end of the polymer, comprises an amino group according to structure —NH—.

According to one preferred embodiment of the present invention, this functional group A is a group having the structure R′—NH— where R′ is hydrogen or a alkyl, cycloalkyl, aryl, aralkyl, arylcycloalkyl, alkaryl or cycloalkylaryl residue where the cycloalkyl, aryl, aralkyl, arylcycloalkyl, alkaryl or cycloalkylaryl residue may be linked directly to the NH group or, according to another embodiment, may be linked by an oxygen bridge to the NH group. The alkyl, cycloalkyl, aryl, aralkyl, arylcycloalkyl, alkaryl, or cycloalkylaryl residues may be suitably substituted. As preferred substituents, halogenes such as F, Cl or Br may be mentioned. Especially preferred residues R′ are hydrogen, alkyl and alkoxy groups, and even more preferred are hydrogen and unsubstituted alkyl and alkoxy groups.

Among the alkyl and alkoxy groups, groups with 1, 2, 3, 4, 5, or 6 C atoms are preferred. More preferred are methyl, ethyl, propyl, isopropyl, methoxy, ethoxy, propoxy, and isopropoxy groups. Especially preferred are methyl, ethyl, methoxy, ethoxy, and particular preference is given to methyl or methoxy.

Therefore, the present invention also relates to a method and a conjugate as described above wherein R′ is hydrogen or a methyl or a methoxy group.

According to another preferred embodiment of the present invention, the functional group A has the structure R′—NH—R″— where R″ preferably comprises the structure unit —NH— and/or the structure unit —(C=G)- where G is O or S, and/or the structure unit —SO₂—. According to more preferred embodiments, the functional group R″ is selected from the group consisting of

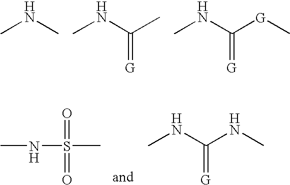

where, if G is present twice, it is independently O or S.

Therefore, preferred functional groups A comprising an amino group —NH₂, are, e.g.,

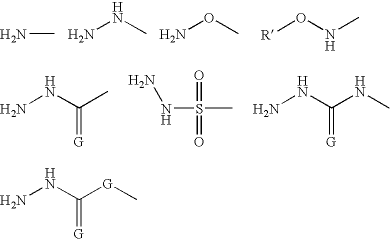

wherein G is O or S and, if present twice, independently O or S, and R′ is methyl.

Especially preferred functional groups A comprising an amino group are aminooxy groups

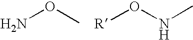

H₂N—O— being particularly preferred, and the hydrazido group

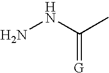

where G is preferably O.

Therefore, the present invention also relates to a method as described above, wherein the functional group Z of the protein is an aldehyde group or a keto group, and the functional group A is an aminooxy group or a hydrazido group. According to an especially preferred embodiment of the present invention, A is an aminooxy group.

Thus, the present invention also relates to a conjugate, as described above, wherein the functional group Z of the protein is an aldehyde group or a keto group, and the functional group A is an aminooxy group or a hydrazido group. According to an especially preferred embodiment of the present invention, A is an aminooxy group.

When reacting the aminooxy group of the polymer or polymer derivative with the aldehyde group or keto group of the protein, an oxime linkage is formed.

Therefore, the present invention also relates to a conjugate as described above, wherein the covalent linkage between the protein and the polymer or polymer derivative is an oxime linkage formed by the reaction of functional group Z of the protein, said functional group Z being an aldehyde group or a keto group, and functional group A of the polymer or polymer derivative, said functional group A being an aminooxy group.

When reacting the hydrazido group of the polymer or polymer derivative with the aldehyde group or keto group of the protein, a hydrazone linkage is formed.

Therefore, the present invention also relates to a conjugate as described above, wherein the covalent linkage between the protein and the polymer or polymer derivative is a hydrazone linkage formed by the reaction of functional group Z of the protein, said functional group Z being an aldehyde group or a keto group, and functional group A of the polymer or polymer derivative, said functional group A being a hydrazido group.

In order to introduce functional group A into the polymer, no specific restrictions exist given that a polymer derivative results comprising functional group A.

According to a preferred embodiment of the present invention, the functional group A is introduced in to the polymer by reacting the polymer with an at least bifunctional compound, one functional group of which is capable of being reacted with at least one functional group of the polymer, and at least one other functional group of the at least bifunctional compound being functional group A or being capable of being chemically modified to give functional group A.

According to a still further preferred embodiment, the polymer is reacted with the at least bifunctional compound at its optionally oxidized reducing end.

In case the polymer is reacted with its non-oxidized reducing end, the polymer preferably has the constitution

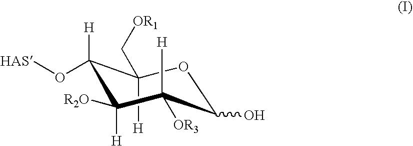

wherein in formula (I), the aldehyde form of the non-oxidized reducing end is included.

In case the polymer is reacted with its oxidized reducing end, the polymer preferably has the constitution according to formula (IIa)

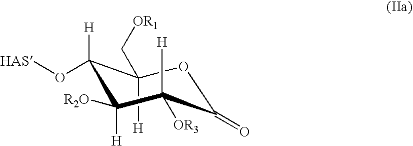

and/or according to formula (IIb)

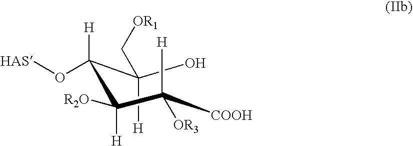

The oxidation of the reducing end of the polymer, preferably hydroxyethyl starch, may be carried out according to each method or combination of methods which result in compounds having the above-mentioned structures (IIa) and/or (IIb).

Although the oxidation may be carried out according to all suitable method or methods resulting in the oxidized reducing end of hydroxyalkyl starch, it is preferably carried out using an alkaline iodine solution as described, e.g., in DE 196 28 705 A1 the respective contents of which (example A, column 9, lines 6 to 24) is incorporated herein by reference.

As functional group of the at least bifunctional compound which is capable of being reacted with the optionally oxidized reducing end of the polymer, each functional group may be used which is capable of forming a chemical linkage with the optionally oxidized reducing end of the hydroxyalkyl starch.

According to a preferred embodiment of the present invention, this functional group comprises the chemical structure —NH—.

Therefore, the present invention also relates to a method and a conjugate as described above wherein the functional group of the at least bifunctional compound, said functional group being capable of being reacted with the optionally oxidized reducing end of the polymer, comprises the structure —NH—.

According to one preferred embodiment of the present invention, this functional group of the at least bifunctional compound is a group having the structure R′—NH— where R′ is hydrogen or a alkyl, cycloalkyl, aryl, aralkyl, arylcycloalkyl, alkaryl or cycloalkylaryl residue where the cycloalkyl, aryl, aralkyl, arylcycloalkyl, alkaryl or cycloalkylaryl residue may be linked directly to the NH group or, according to another embodiment, may be linked by an oxygen bridge to the NH group. The alkyl, cycloalkyl, aryl, aralkyl, arylcycloalkyl, alkaryl, or cycloalkylaryl residues may be suitably substituted. As preferred substituents, halogenes such as F, Cl or Br may be mentioned. Especially preferred residues R′ are hydrogen, alkyl and alkoxy groups, and even more preferred are hydrogen and unsubstituted alkyl and alkoxy groups.

Among the alkyl and alkoxy groups, groups with 1, 2, 3, 4, 5, or 6 C atoms are preferred. More preferred are methyl, ethyl, propyl, isopropyl, methoxy, ethoxy, propoxy, and isopropoxy groups. Especially preferred are methyl, ethyl, methoxy, ethoxy, and particular preference is given to methyl or methoxy.

Therefore, the present invention also relates to a method and a conjugate as described above wherein R′ is hydrogen or a methyl or a methoxy group.

According to another preferred embodiment of the present invention, the functional group of the at least bifunctional compound has the structure R′—NH—R″— where R″ preferably comprises the structure unit —NH— and/or the structure unit —(C=G)- where G is O or S, and/or the structure unit —SO₂—. According to more preferred embodiments, the functional group R″ is selected from the group consisting of

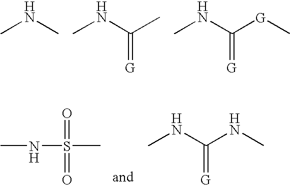

where, if G is present twice, it is independently O or S.

Therefore, the present invention also relates to a method and a conjugate as described above, wherein the functional group of the at least bifunctional compound, said functional group being capable of being reacted with the optionally oxidized reducing end of the polymer, is selected from the group consisting of

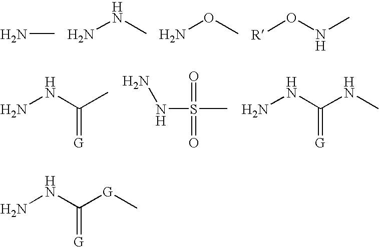

wherein G is O or S and, if present twice, independently O or S, and R′ is methyl.

According to an even more preferred embodiment of the present invention, the functional group of the at least bifunctional compound, said functional group being capable of being reacted with the optionally oxidized reducing end of the polymer and comprising an amino group, is an aminooxy groups

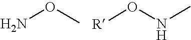

H₂N—O— being particularly preferred, or the hydrazido group

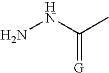

wherein G is preferably O.

Therefore, the present invention also relates to a method and a conjugate as described above, wherein the functional group Z of the protein is an aldehyde group or a keto group, and the functional group of the at least bifunctional compound, said functional group being capable of being reacted with the optionally oxidized reducing end of the polymer, is an aminooxy group or a hydrazido group, preferably an aminooxy group.

Thus, the present invention also relates to a conjugate, as described above, wherein the functional group Z of the protein is an aldehyde group or a keto group, and the functional group of the at least bifunctional compound, said functional group being capable of being reacted with the optionally oxidized reducing end of the polymer, is an aminooxy group or a hydrazido group, preferably an aminooxy group.

According to a still further preferred embodiment of the present invention, the at least bifunctional compound is reacted with the polymer at its non-oxidized reducing end.

According to yet another preferred embodiment of the present invention, the at least bifunctional compound which is reacted with the optionally oxidized reducing end of the polymer, comprises functional group A.

The at least bifunctional compound may be reacted with the polymer first to give a polymer derivative which is subsequently reacted with the protein via functional group A. It is also possible to react the at least bifunctional compound via functional group A with the protein first to give a protein derivative which is subsequently reacted with the polymer via at least one functional group of the at least bifunctional compound residue comprised in the protein derivative.

According to a preferred embodiment of the present invention, the at least bifunctional compound is reacted with the polymer first.

Therefore, the present invention relates to a method and a conjugate as described above, said method further comprising reacting the polymer at its non-oxidized reducing end with an at least bifunctional linking compound comprising a functional group capable of reacting with the non-oxidized reducing end of the polymer and a group A, prior to the reaction of the polymer derivative comprising A and the protein comprising Z.

The functional group of the at least bifunctional linking compound which is reacted with the polymer and the functional group A of the at least bifunctional linking compound which is reacted with functional group Z of the protein may be separated by any suitable spacer. Among others, the spacer may be an optionally substituted, linear, branched and/or cyclic hydrocarbon residue. Generally, the hydrocarbon residue has up to 60, preferably up to 40, more preferably up to 20, more preferably up to 10, more preferably up to 6 and especially preferably up to 4 carbon atoms. If heteroatoms are present, the separating group comprises generally from 1 to 20, preferably from 1 to 8, more preferably 1 to 6, more preferably 1 to 4 and especially preferably from 1 to 2 heteroatoms. As heteroatom, O is preferred. The hydrocarbon residue may comprise an optionally branched alkyl chain or an aryl group or a cycloalkyl group having, e.g., from 5 to 7 carbon atoms, or be an aralkyl group, an alkaryl group where the alkyl part may be a linear and/or cyclic alkyl group. According to an even more preferred embodiment of the present invention, the functional groups are separated by a linear hydrocarbon chain having 4 carbon atoms. According to another preferred embodiment of the present invention, the functional groups are separated by a linear hydrocarbon chain having 4 carbon atoms and at least one, preferably one heteroatom, particularly preferably an oxygen atom.

According to a further preferred embodiment, the at least bifunctional linking compound is a homobifunctional linking compound. Therefore, the present invention also relates to a method of producing a conjugate as described above, wherein the at least bifunctional linking compound is a homobifunctional compound.

Thus, with regard to the above mentioned preferred functional groups of the linking compound, said homobifunctional linking compound preferably comprises either two aminooxy groups H₂N—O— or two aminooxy groups R′—O—NH— or two hydrazido groups H₂N—NH—(C=G)-, the aminooxy groups H₂N—O— and the hydrazido groups H₂N—NH—(C═O)— being preferred, and the aminooxy groups H₂N—O— being especially preferred.

Among all conceivable homobifunctional compounds comprising two hydrazido groups H₂N—NH—(C═O)—, hydrazides are preferred where the two hydrazido groups are separated by a hydrocarbon residue having up to 60, preferably up to 40, more preferably up to 20, more preferably up to 10, more preferably up to 6 and especially preferably up to 4 carbon atoms. More preferably, the hydrocarbon residue has 1 to 4 carbon atoms such as 1, 2, 3, or 4 carbon atoms. Most preferably, the hydrocarbon residue has 4 carbon atoms. Therefore, a homobifunctional compound according to formula

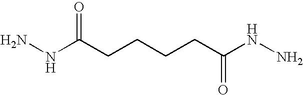

is preferred.

According to an even more preferred embodiment of the present invention, the bifunctional linking compound is carbohydrazide

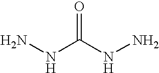

As described above, the present invention also relates to a method and a conjugate as described above, wherein the at least bifunctional linking compound is a homobifunctional compound and comprises two aminooxy groups. Hence, the present invention also relates to a method and a conjugate as described above, wherein the at least bifunctional linking compound is a homobifunctional compound and comprises two aminooxy groups H₂N—O—.

As described above, the polymer is preferably reacted at its reducing end which is not oxidized prior to the reaction with the bifunctional linking compound. Therefore, reacting the preferred homobifunctional compound comprising two aminooxy groups H₂N—O— with the polymer results in a polymer derivative comprising an oxime linkage.

Therefore, since functional group Z of the protein is an aldehyde or a keto group which is preferably reacted with an aminooxy group of the polymer derivative, the present invention also relates to a conjugate as described above, said conjugate comprising the polymer and the protein, each being covalently linked to a linking compound by an oxime or a cyclic animal linkage.

Among all conceivable homobifunctional compounds comprising two aminooxy groups H₂N—O—, bifunctional compounds are preferred where the two aminooxy groups are separated by a hydrocarbon residue having from 1 to 60, preferably from 1 to 40, more preferably from 1 to 20, more preferably from 1 to 10, more preferably from 1 to 6 and especially preferably 1 to 4 carbon atoms. More preferably, the hydrocarbon residue has 1 to 4 carbon atoms such as 1, 2, 3, or 4 carbon atoms. Most preferably, the hydrocarbon residue has 4 carbon atoms. Even more preferably, the hydrocarbon residue has at least one heteroatom, more preferably one heteroatom, and most preferably one oxygen atom. The compound O-[2-(2-aminooxy-ethoxy)-ethyl]hydroxyl amine according to formula

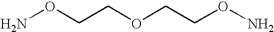

is especially preferred.

Therefore, the present invention relates to a conjugate as described above, said conjugate having a constitution according to formula

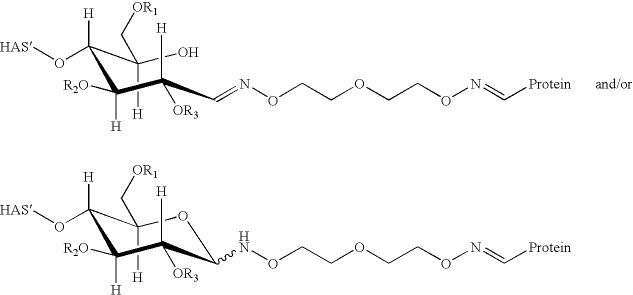

HAS′ preferably being HES′. Particularly preferred hydroxyethyl starches are, e.g., hydroxethyl starches having a mean molecular weight of about 10 kD and a DS of about 0.4 or hydroxethyl starch having a mean molecular weight of about 10 kD and a DS of about 0.7 or hydroxethyl starch having a mean molecular weight of about 12 kD and a DS of about 0.4 or hydroxethyl starch having a mean molecular weight of about 12 kD and a DS of about 0.7 or hydroxethyl starch having a mean molecular weight of about 18 kD and a DS of about 0.4 or hydroxethyl starch having a mean molecular weight of about 18 kD and a DS of about 0.7 or hydroxethyl starch having a mean molecular weight of about 50 kD and a DS of about 0.4 or hydroxethyl starch having a mean molecular weight of about 50 kD and a DS of about 0.7.

The reaction of the polymer at its non-oxidized reducing end with the linking compound, especially in the case said linking compound is a homobifunctional linking compound comprising two aminooxy groups H₂N—O—, is preferably carried out in an aqueous system.

The term “aqueous system” as used in the context of the present invention refers to a solvent or a mixture of solvents comprising water in the range of from at least 10% per weight, preferably at least 50% per weight, more preferably at least 80% per weight, even more preferably at least 90% per weight or up to 100% per weight, based on the weight of the solvents involved. The preferred reaction medium is water.

According to another embodiment, at least one other solvent may be used in which HAS, preferably HES is soluble. Examples of these solvents are, e.g., DMF, dimethylacetamide or DMSO.

As far as the temperatures which are applied during the reaction are concerned, no specific limitations exist given that the reaction results in the desired polymer derivative.

In case the polymer is reacted with the homobifunctional linking compound comprising two aminooxy groups H₂N—O—, preferably O-[2-(2-aminooxy-ethoxy)-ethyl]hydroxy amine, the temperature is preferably in the range of from 0 to 45° C., more preferably in the range of from 4 to 30° C. and especially preferably in the range of from 15 to 25° C.

The reaction time for the reaction of the polymer with the homobifunctional linking compound comprising two aminooxy groups H₂N—O—, preferably O-[2-(2-aminooxy-ethoxy)-ethyl]hydroxyl amine, may be adapted to the specific needs and is generally in the range of from 1 h to 7 d, preferably in the range of from 1 h to 3 d and more preferably of from 2 h to 48 h.

The pH value for the reaction of the polymer with the homobifunctional linking compound comprising two aminooxy groups H₂N—O—, preferably O-[2-(2-aminooxy-ethoxy)-ethyl]hydroxyl amine, may be adapted to the specific needs such as the chemical nature of the reactants. The pH value is preferably in the range of from 4.5 to 9.5, more preferably in the range of from 4.5 to 6.5.

Specific examples of above mentioned reaction conditions are, e.g., a reaction temperature of about 25° C. and a pH of about 5.5.

The suitable pH value of the reaction mixture may be adjusted by adding at least one suitable buffer. Among the preferred buffers, sodium acetate buffer, phosphate or borate buffers may be mentioned.

Once the polymer derivative comprising the polymer and the bifunctional linking compound linked thereto is formed, it may be isolated from the reaction mixture by at least one suitable method. If necessary, the polymer derivative may be precipitated prior to the isolation by at least one suitable method.

If the polymer derivative is precipitated first, it is possible, e.g., to contact the reaction mixture with at least one solvent or solvent mixture other than the solvent or solvent mixture present in the reaction mixture at suitable temperatures. According to a particularly preferred embodiment of the present invention where an aqueous medium, preferably water is used as solvent, the reaction mixture is contacted with 2-propanol, at a temperature, preferably in the range of from −20 to +50° C. and especially preferably in the range of from −20 to 25° C.

Isolation of the polymer derivative may be carried out by a suitable process which may comprise one or more steps. According to a preferred embodiment of the present invention, the polymer derivative is first separated off the reaction mixture or the mixture of the reaction mixture with, e.g., aqueous 2-propanol mixture, by a suitable method such as centrifugation or filtration. In a second step, the separated polymer derivative may be subjected to a further treatment such as an after-treatment like dialysis, centrifugal filtration or pressure filtration, ion exchange chromatography, reversed phase chromatography, HPLC, MPLC, gel filtration and/or lyophilisation. According to an even more preferred embodiment, the separated polymer derivative is first dialysed, preferably against water, and then lyophilized until the solvent content of the reaction product is sufficiently low according to the desired specifications of the product. Lyophilisation may be carried out at temperature of from 20 to 35° C., preferably of from 20 to 30° C.

The thus isolated polymer derivative is then further reacted, via functional group A, with the functional group Z of the protein, Z being an aldehyde group or a keto group. In the especially preferred case that A is an aminooxy group H₂N—O— to give an oxime linkage between polymer derivative and protein, the reaction is preferably carried out in an aqueous medium, preferably water, at a preferred temperature in the range of from 0 to 40° C., more preferably from 4 to 25° C. and especially preferably from 15 to 25° C. The pH value of the reaction medium is preferably in the range of from 4 to 10, more preferably in the range of from 5 to 9 and especially preferably in the range of from 5 to 7. The reaction time is preferably in the range of from 1 to 72 h, more preferably in the range of from 1 to 48 h and especially preferably in the range of from 4 to 24 h.

The conjugate may be subjected to a further treatment such as an after-treatment like dialysis, centrifugal filtration or pressure filtration, ion exchange chromatography, reversed phase chromatography, HPLC, MPLC, gel filtration and/or lyophilisation.

According to another embodiment of the present invention, the functional group Z of the protein is an amino group. Therefore, the present invention relates to a method and a conjugate as described above, wherein the functional group Z of the protein is an amino group.

According to a further preferred embodiment of the present invention, the functional group A to be reacted with the functional group Z being an amino group is a reactive carboxy group. Therefore, the present invention also relates to a method and a conjugate as described above, wherein the functional group Z is an amino group and the functional group A of the polymer or the polymer derivative is a reactive carboxy group.

According to a first preferred embodiment of the present invention, the reactive carboxy group is introduced into the polymer by selectively oxidizing the polymer at its reducing end.

Therefore, the polymer into which the reactive carboxy group is introduced preferably has the constitution according to formula (IIa)

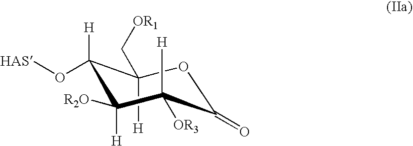

and/or according to formula (IIb)

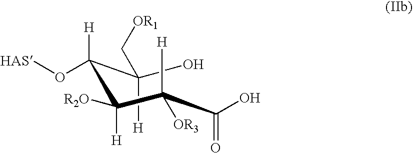

The oxidation of the reducing end of the polymer according to formula (I)

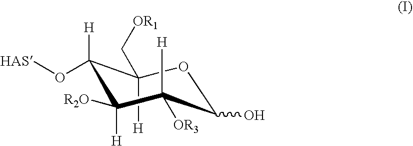

preferably hydroxyethyl starch, may be carried out according to each method or combination of methods which result in compounds having the above-mentioned structures (IIa) and/or (IIb).

Although the oxidation may be carried out according to all suitable method or methods resulting in the oxidized reducing end of hydroxyalkyl starch, it is preferably carried out using an alkaline iodine solution as described, e.g., in DE 196 28 705 A1 the respective contents of which (example A, column 9, lines 6 to 24) is incorporated herein by reference.

Introducing the reactive carboxy group into the polymer which is selectively oxidized at its reducing end may carried out by all conceivable methods.

The oxidized polymer may be employed as such or as salt, such as alkali metal salt, preferably as sodium and/or potassium salt.

According to a preferred method of the present invention, the polymer which is selectively oxidized at its reducing end is reacted at the oxidized reducing end with at least one alcohol, preferably with at least one acidic alcohol. Still further preferred are acidic alcohols having a pK_{A} value in the range of from 6 to 12, more preferably of from 7 to 11 at 25° C. The molecular weight of the acidic alcohol is preferably in the range of from 80 to 500 g/mole, more preferably of from 90 to 300 g/mole and especially preferably of from 100 to 200 g/mole.

Suitable acidic alcohols are all alcohols H—O—R_{A} having an acidic proton and are capable of being reacted with the oxidized polymer to give the respective reactive polymer ester, preferably according to the formula

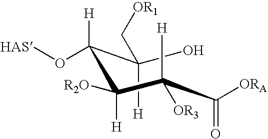

still more preferably according to formula

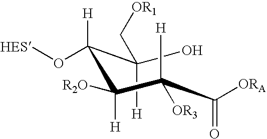

Preferred alcohols are N-hydroxy succinimides such as N-hydroxy succinimide or Sulfo-N-hydroxy succinimide, suitably substituted phenols such as p-nitrophenol, o,p-dinitrophenol, o,o′-dinitrophenol, trichlorophenol such as 2,4,6-trichlorophenol or 2,4,5-trichlorophenol, trifluorophenol such as 2,4,6-trifluorophenol or 2,4,5-trifluorophenol, pentachlorophenol, pentafluorophenol, or hydroxyazoles such as hydroxy benzotriazole. Especially preferred are N-hydroxy succinimides, with N-hydroxy succinimide and Sulfo-N-hydroxy succinimide being especially preferred. All alcohols may be employed alone or as suitable combination of two or more thereof. In the context of the present invention, it is also possible to employ a compound which releases the respective alcohol, e.g. by adding diesters of carbonic acid.

Therefore, the present invention also relates to a method and a conjugate as described above, wherein the polymer which is selectively oxidised at its reducing end is activated by reacting the oxidised polymer with an acidic alcohol, preferably with N-hydroxy succinimide and/or Sulfo-N-hydroxy succinimide.

According to a still further preferred embodiment of the present invention, the polymer which is selectively oxidized at its reducing end is reacted at the oxidized reducing end with at least one carbonic diester R_{B}—O—(C═O)—O—R_{C}, wherein R_{B} and R_{C} may be the same or different. Preferably, this method gives reactive polymers according to the formula

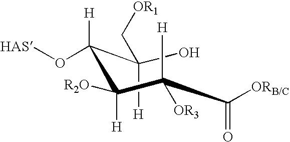

wherein HAS′ is preferably HES′.

As suitable carbonic diester compounds, compounds may be employed whose alcohol components are independently N-hydroxy succinimides such as N-hydroxy succinimide or Sulfo-N-hydroxy succinimide, suitably substituted phenols such as p-nitrophenol, o,p-dinitrophenol, o,o′-dinitrophenol, trichlorophenol such as 2,4,6-trichlorophenol or 2,4,5-trichlorophenol, trifluorophenol such as 2,4,6-trifluorophenol or 2,4,5-trifluorophenol, pentachlorophenol, pentafluorophenol, or hydroxyazoles such as hydroxy benzotriazole. Especially preferred are N,N′-disuccinimidyl carbonate and Sulfo-N,N′-disuccinimidyl carbonate, with N,N′-disuccinimidyl carbonate being especially preferred.

Therefore, the present invention also relates to a method and a conjugate as described above, wherein the polymer which is selectively oxidised at its reducing end is activated by reacting the oxidised polymer with N,N′-disuccinimidyl carbonate.

The acidic alcohol is reacted with the oxidized polymer or the salt of the oxidized polymer at a molar ratio of acidic alcohol:polymer preferably of from 5:1 to 50:1, more preferably of from 8:1 to 20:1, at a preferred reaction temperature of from 2 to 40° C., more preferably of from 10 to 30° C. and especially preferably of from 15 to 25° C. The reaction time is preferably in the range of from 1 to 10 h, more preferably of from 2 to 5 h, more preferably of from 2 to 4 h and particularly of from 2 to 3 h.

The carbonic diester compound is reacted with the oxidized polymer or the salt of the oxidized polymer at a molar ratio of diester compound:polymer preferably of from 1:1 to 3:1, more preferably of from 1:1 to 1.5:1. The reaction time is preferably in the range of from 0.1 to 12 h, more preferably of from 0.2 to 6 h, more preferably of from 0.5 to 2 h and particularly of from 0.75 to 1.25 h.

According to a preferred embodiment of the present invention, reacting the oxidized polymer with acidic alcohol and/or carbonic diester is carried out in at least one aprotic solvent, particularly preferably in an anhydrous aprotic solvent having a water content of not more than 0.5 percent by weight, preferably of not more than 0.1 percent by weight. Suitable solvents are, among others, dimethyl sulfoxide (DMSO), N-methylpyrrolidone, dimethyl acetamide (DMA), dimethyl formamide (DMF) and mixtures of two or more thereof. The reaction temperatures are preferably in the range of from 2 to 40° C., more preferably of from 10 to 30° C.

For reacting the oxidized polymer with the at least one acidic alcohol, at least one additional activating agent is employed.

Suitable activating agents are, among others, carbonyldiimidazole, carbodiimides such as diisopropyl carbodimide (DIC), dicyclohexyl carbodiimides (DCC), 1-ethyl-3-(3-dimethylaminopropyl)carbodiimide (EDC), with dicyclohexyl carbodiimides (DCC) and 1-ethyl-3-(3-dimethylaminopropyl)carbodiimide (EDC) being especially preferred.

Therefore, the present invention also relates to a method and a conjugate as described above, where the polymer which is oxidized at its reducing end, is reacted with an acidic alcohol in the presence of an additional activating agent to give the reactive polymer ester.

According to an especially preferred embodiment of the present invention, the reaction of the oxidized polymer with carbonic diester and/or acidic alcohol is carried out at a low base activity which may be determined by adding the reaction mixture to water with a volume ratio of water to reaction mixture of 10:1. Prior to the addition, the water which comprises essentially no buffer, has a pH value of 7 at 25° C. After the addition of the reaction mixture and by measuring the pH value, the base activity of the reaction mixture is obtained, having a value of preferably not more than 9.0, more preferably of nor more than 8.0 and especially preferably of not more than 7.5.

According to preferred embodiment of the present invention, the oxidized polymer is reacted with N-hydroxy succinimide in dry DMA in the absence of water with EDC to selectively give the polymer N-hydroxy succinimide ester according to the formula

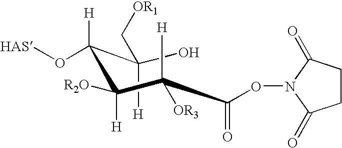

more preferably with HAS′ being HES′.

Surprisingly, this reaction does not give by-products resulting from reactions of EDC with OH groups of HES, and the rearrangement reaction of the O-acyl isourea formed by EDC and the oxidized polymer to the respective N-acyl urea is surprisingly suppressed.

According to another preferred embodiment of the present invention, the oxidized polymer is reacted with N,N′-disuccinimidyl carbonate in anhydrous DMF and in the absence of an activating agent to selectively give the polymer N-hydroxy succinimide ester according to the formula

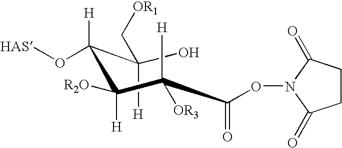

more preferably with HAS′ being HES′.

The reactive polymer as described above is preferentially further reacted with at least one amino group of the protein to give an amide linkage. According to a preferred embodiment of the present invention, the reactive polymer is reacted with one amino group of the protein.

Therefore, the present relates to a conjugate preferably having a constitution according to the formula

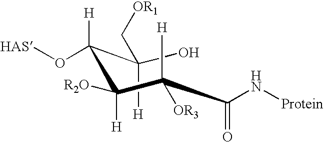

wherein the N atom of the amide linkage is derived from an amino group of the protein, more preferably with HAS′ being HES′, the hydroxyethyl starch preferably being hydroxethyl starch having a mean molecular weight of about 10 kD and a DS of about 0.4 or hydroxethyl starch having a mean molecular weight of about 10 kD and a DS of about 0.7 or hydroxethyl starch having a mean molecular weight of about 12 kD and a DS of about 0.4 or hydroxethyl starch having a mean molecular weight of about 12 kD and a DS of about 0.7 or hydroxethyl starch having a mean molecular weight of about 18 kD and a DS of about 0.4 or hydroxethyl starch having a mean molecular weight of about 18 kD and a DS of about 0.7 or hydroxethyl starch having a mean molecular weight of about 50 kD and a DS of about 0.4 or hydroxethyl starch having a mean molecular weight of about 50 kD and a DS of about 0.7.

The reaction of the reactive polymer with the protein may be carried out by combining the reaction mixture of the preparation of the reactive polymer, i.e. without isolation of the reactive polymer, comprising at least 10, more preferably at least 30 and still more preferably at least 50 percent by weight reactive polymer, with an aqueous solution of the protein. Preferred aqueous solutions of the protein comprises of from 0.05 to 10, more preferably of from 0.5 to 5 and especially preferably of from 0.5 to 2 percent by weight protein at a preferred pH of from 5.0 to 9.0, more preferably of from 6.0 to 9.0 and especially preferably of from 7.5 to 8.5.

According to the present invention, it is also possible to purify the reactive polymer by at least one, preferably multiple precipitation with at least one suitable precipitation agent such as anhydrous ethanol, isopropanol and/or acetone to give a solid comprising at least 10, more preferably at least 30 and still more preferably at least 50 percent by weight reactive polymer.

The purified reactive polymer may be added to the aqueous solution of the protein. It is also possible to add a solution of the purified reactive polymer to the aqueous solution of the protein.

According to a preferred embodiment of the present invention, the reaction of the reactive polymer with the protein to give an amide linkage is carried out at a temperature of from 2 to 40° C., more preferably of from 5 to 35° C. and especially of from 10 to 30° C. and a preferred pH of from 7.0 to 9.0, preferably of from 7.5 to 9.0 and especially preferably of from 7.5 to 8.5, at a preferred reaction time of from 0.1 to 12 h, more preferably of from 0.5 to 5 h, more preferably of from 0.5 to 3 h, still more preferably of from 0.5 to 72 h and especially preferably of from 0.5 to 1 h, the molar ratio of reactive polymer ester:protein being preferably of from 1:1 to 70:1, more preferably of from 5:1 to 50:1 and especially preferably of from 10:1 to 50:1.

According to another embodiment of the present invention, the polymer which is selectively oxidized at its reducing end is reacted at the oxidized reducing end with an azolide such as carbonyldiimidazole or carbonyl dibenzimidazole to give a polymer having a reactive carboxy group. In the case of carbonyldiimidazole, a reactive polymer derivative according to formula

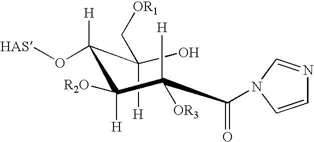

results, wherein HAS′ is preferably HES′. The imidazolide resulting from the reaction of the polymer with the azolide may be preferentially reacted with an amino group of the protein to give an amide linkage. Also possible is a reaction, if present, with a hydroxy group of the protein to give an ester linkage, or with a thio group of the protein to give a thioester linkage, or, if present, with a carboxy group of the protein to give a —(C═O)—O—(C═O)— linkage.

According to another embodiment of the present invention, the polymer having a reactive carboxy group A resulting from the reaction of the selectively oxidized reducing end of the polymer with one of the above-mentioned compounds, preferably with at least one of the acidic alcohols and/or at least one of the carbonic diester compounds, may be linked to the functional group Z of the protein via at least one linker compound. In case a linker compound is used, said compound is an at least bifunctional compound having at least one functional group F₁ capable of being reacted with the functional group A of the polymer derivative, and at least one functional group F₂ being capable of being reacted with the functional group Z of the protein or a functional group F₂ being capable of being chemically modified to be reacted with the functional group Z of the protein. The chemical modification may be, e.g., a reaction of the functional group F₂ with a functional group F₃ of a further linker compound or an oxidation or a reduction of a suitable functional group F₂. In case at least one linker compound is used, the reaction is not restricted to the amino group of the protein but, depending on the chemical nature of the functional groups of the linker compound or linker compounds, may be used to form a linkage with each suitable functional group of the protein, such as a carboxy group, a reactive carboxy group, an aldehyde group, a keto group, a thio group, an amino group or a hydroxy group. In case two linker compounds are used, a first linker compound is employed having at least one functional group F₁ being capable of being reacted with the reactive carboxy group A of the polymer, such as an amino group, a thio group, a hydroxy group, or a carboxy group. Moreover, the first linker compound has at least one other functional group F₂ which is capable of being reacted with at least one functional group F₃ of the second linker compound. As to functional group F₂, the following functional groups are to be mentioned, among others:
C—C-double bonds or C—C-triple bonds or aromatic C—C-bonds;
the thio group or the hydroxy groups;
alkyl sulfonic acid hydrazide, aryl sulfonic acid hydrazide;
1,2-dioles;
1,2-aminoalcohols;
1,2 amino-thioalcohols;
azides;
the amino group —NIH2 or derivatives of the amino groups comprising the structure unit —NH— such as aminoalkyl groups, aminoaryl group, aminoaralkyl groups, or alkarlyamino groups;
the hydroxylamino group —O—NH₂, or derivatives of the hydroxylamino group comprising the structure unit —O—NH—, such as hydroxylalkylamino groups, hydroxylarylamino groups, hydroxylaralkylamino groups, or hydroxalalkarylamino groups;
alkoxyamino groups, aryloxyamino groups, aralkyloxyamino groups, or alkaryloxyamino groups, each comprising the structure unit —NH—O—;
residues having a carbonyl group, -Q-C(=G)-M, wherein G is O or S, and M is, for example,
   —OH or —SH;
   an alkoxy group, an aryloxy group, an aralkyloxy group, or an alkaryloxy group;
   an alkylthio group, an arylthio group, an aralkylthio group, or an alkarylthio group;
   an alkylcarbonyloxy group, an arylcarbonyloxy group, an aralkylcarbonyloxy group, an alkarylcarbonyloxy group;
   activated esters such as esters of hydroxyl amines having imid structure such as N-hydroxysuccinimide or having a structure unit O—N where N is part of a heteroaryl compound or, with G=O and Q absent, such as aryloxy compounds with a substituted aryl residue such as pentafluorophenyl, paranitrophenyl or trichlorophenyl;
wherein Q is absent or NH or a heteroatom such as S or O;
   —NH—NH₂, or —NH—NH—;
   —NO₂;
   the nitril group;
   carbonyl groups such as the aldehyde group or the keto group;
   the carboxy group;
   the —N═C═O group or the —N═C═S group;
   vinyl halide groups such as the vinyl iodide or the vinyl bromide group or triflate;
   —C≡C—H;
   —(C═NH₂Cl)—OAlkyl
   groups —(C═O)—CH₂-Hal wherein Hal is Cl, Br, or I;
   —CH═CH—SO₂—;
   a disulfide group comprising the structure —S—S—;
   the group

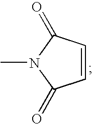

the group

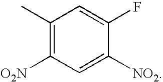

wherein F₃ is a group capable of forming a chemical linkage with one of the above-mentioned groups and is preferably selected from the above-mentioned groups. Moreover, the second linker compound has at least one functional group which is capable of being reacted with the functional group Z of the protein, which is, e.g., an amino group, a thio group, a carboxy group, a reactive carboxy group, an aldehyde group, a keto group, or a hydroxy group. In case one linking compound is used to covalently link the polymer and the protein, the polymer can be reacted with the linking compound and the resulting polymer derivative is reacted with the protein, or the protein can be reacted with the linking compound and the resulting protein derivative is reacted with the polymer. In case two linking compounds L1 and L2 are used, it is possible to react the polymer with L1, react the resulting polymer derivative with L2 and react the resulting polymer derivative with the protein, or to react the protein with L2, react the resulting protein derivative with L1 and react the resulting protein derivative with the polymer. It is also possible to react the polymer with L1 and react the protein with L2 and react the polymer derivative with the protein derivative. Furthermore, it is possible to react L1 with L2, react the resulting compound with the polymer and the resulting polymer derivative with the protein. Furthermore, it is possible to react L1 with L2, react the resulting compound with the protein and the resulting protein derivative with the polymer.

According to a second preferred embodiment of the present invention regarding the introduction of a reactive carboxy group into the polymer, the reactive carboxy group is introduced into the polymer whose reducing end is not oxidized, by reacting at least one hydroxy group of the polymer with a carbonic diester.

Therefore, the present invention also relates to a method and a conjugate as described above, wherein A is a reactive carboxy group, and wherein A is introduced in the polymer whose reducing end is not oxidized, by reacting at least one hydroxy group of the polymer with at least one carbonic diester carbonic diester R_{B}—O—(C═O)—O—R_{C}, wherein R_{B} and R_{C} may be the same or different.

According to another embodiment of the present invention, the polymer whose reducing end is not oxidized, is reacted at least one hydroxy group with an azolide such as carbonyldiimidazole, carbonyl-di-(1,2,4-triazole) or carbonyl dibenzimidazol to give a polymer having a reactive carboxy group.

As suitable carbonic diester compounds, compounds may be employed whose alcohol components are independently N-hydroxy succinimides such as N-hydroxy succinimide or Sulfo-N-hydroxy succinimide, suitably substituted phenols such as p-nitrophenol, o,p-dinitrophenol, o,o′-dinitrophenol, trichlorophenol such as 2,4,6-trichlorophenol or 2,4,5-trichlorophenol, trifluorophenol such as 2,4,6-trifluorophenol or 2,4,5-trifluorophenol, pentachlorophenol, pentafluorophenol, or hydroxyazoles such as hydroxy benzotriazole.

Especially preferred are symmetrical carbonic diester compounds, R_{B} and R_{C} thus being the same. The alcohol component of the carbonic diester is preferably selected from the group consisting of N-hydroxy succinimide, sulfonated N-hydroxy succinimide, N-hydroxy benzotriazole, and nitro- and halogen-substituted phenols. Among others, nitrophenol, dinitrophenol, trichlorophenol, trifluorophenol, pentachlorophenol, and pentafluorophenol are preferred. Especially preferred are N,N′-disuccinimidyl carbonate and Sulfo-N,N′-disuccinimidyl carbonate, with N,N′-disuccinimidyl carbonate being especially preferred.

Therefore, the present invention also relates to a hydroxyalkyl starch derivative and a method of producing same, preferably a hydroxyethyl starch derivative, wherein at least one hydroxy group, preferably at least two hydroxy groups of said starch have been reacted with a carbonic diester compound to give the respective reactive ester.

According to a preferred embodiment of the present invention, the reaction of the polymer whose reducing end is not oxidized, with the at least one carbonic diester compound is carried out at a temperature of from 2 to 40° C., more preferably of from 10 to 30° C. and especially of from 15 to 25° C. and at a preferred reaction time of from 0.5 to 5 h, more preferably of from 1 to 3 h, and especially preferably of from 2 to 3 h.

The molar ratio of carbonic diester and/or azolide, preferably carbonic diester compound:polymer depends on the degree of substitution of the polymer regarding the number of hydroxy groups reacted with carbonic diester compound relative to the number of hydroxy groups present in the non-reacted polymer.

According to one preferred embodiment of the present invention, the molar ratio of carbonic diester compound:anhydroglucose units are in the range of from 1:2 to 1:1000, more preferably of from 1:3 to 1:100 and especially preferably of from 1:10 to 1:50, to give a degree of substitution in the range of from 0.5 to 0.001, preferably of from 0.33 to 0.01 and especially preferably of from 0.1 to 0.02. The degree of substitution is determined via UV spectroscopy.

According to a preferred embodiment of the present invention, reacting the polymer whose reducing end is not oxidized, with carbonic diester is carried out in at least one aprotic solvent, particularly preferably in an anhydrous aprotic solvent having a water content of not more than 0.5 percent by weight, preferably of not more than 0.1 percent by weight. Suitable solvents are, among others, dimethyl sulfoxide (DMSO), N-methyl pyrrolidone, dimethyl acetamide (DMA), dimethyl formamide (DMF) and mixtures of two or more thereof.

Therefore, the present invention also relates to a method and a conjugate as described above, wherein the reaction of the at least one hydroxy group of the polymer whose reducing end is not oxidised, with the carbonic diester to give a reactive ester group A is carried out in an anhydrous aprotic polar solvent, the solvent preferably being dimethyl acetamide, dimethyl formamide or a mixture thereof.

The reaction of the reactive polymer comprising at least one reactive ester group, preferably at least two reactive ester groups, with the protein to give at least one amide linkage, preferably at least two amide linkages, may be carried out by combining the reaction mixture of the preparation of the reactive polymer, i.e. without isolation of the reactive polymer, comprising at least 5, more preferably at least 10 and still more preferably at least 15 percent by weight reactive polymer, with an aqueous solution of the protein. Preferred aqueous solutions of the protein comprises of from 0.05 to 10, more preferably of from 0.5 to 5 and especially preferably of from 0.5 to 2 percent by weight protein at a preferred pH of from 7.0 to 9., more preferably of from 7.5 to 9 and especially preferably of from 7.5 to 8.5.

According to the present invention, it is also possible to purify the reactive polymer by at least one, preferably by multiple precipitation with at least one suitable precipitation agent such as anhydrous ethanol, isopropanol and/or acetone to give a solid comprising at least 20, more preferably at least 50 and still more preferably at least 80 percent by weight reactive polymer.

The purified reactive polymer may be added to the aqueous solution of the protein. It is also possible to add a solution of the purified reactive polymer to the aqueous solution of the protein.

According to a preferred embodiment of the present invention, the reaction of the reactive polymer with the protein to give at least one, preferably at least two amide linkages is carried out at a temperature of from 2 to 40° C., more preferably of from 5 to 35° C. and especially of from 10 to 30° C. and a preferred pH of from 7.5 to 9.5, preferably of from 7.5 to 9 and especially preferably of from 7.5 to 8.5, at a preferred reaction time of from 0.5 to 5 h, more preferably of from 0.5 to 3 h and especially preferably of from 0.5 to 1 h, the molar ratio of reactive polymer ester:protein being preferably of from 1:1 to 70:1, more preferably of from 5:1 to 50:1 and especially preferably of from 10:1 to 50:1.

According to a preferred embodiment of the present invention, oligo- or multiprotein-substituted polymers are obtained wherein the protein molecules are linked to the polymer via an amide linkage.

The degree of substitution of the protein molecules (PDS) as used in the context of the present invention refers to the portion of glucose moieties linked to a protein with respect to all glucose moieties comprised in HAS, preferably HES.

PDS is in the range of from 0.001 to 1, preferably from 0.005 to 0.5, more preferably from 0.005 to 0.2.

According to another embodiment of the present invention, the polymer having a reactive carboxy group A resulting from the reaction of at least one hydroxy group of the polymer with one of the above-mentioned compounds, preferably with at least one of the carbonic diester compounds, may be linked to the functional group Z of the protein via at least one linker compound. In case a linker compound is used, said compound is an at least bifunctional compound having at least one functional group F₁ capable of being reacted with the functional group A of the polymer derivative, and at least one functional group F₂ being capable of being reacted with the functional group Z of the protein or a functional group F₂ being capable of being chemically modified to be reacted with the functional group Z of the protein. The chemical modification may be, e.g., a reaction of the functional group F₂ with a functional group F₃ of a further linker compound or an oxidation or a reduction of a suitable functional group F₂. In case at least one linker compound is used, the reaction is not restricted to the amino group of the protein but, depending on the chemical nature of the functional groups of the linker compound or linker compounds, may be used to form a linkage with each suitable functional group of the protein, such as a carboxy group, a reactive carboxy group, an aldehyde group, a keto group, a thio group, an amino group or a hydroxy group. In case two linker compounds are used, a first linker compound is employed having at least one functional group F₁ being capable of being reacted with the reactive carboxy group A of the polymer, such as an amino group, a thio group, a hydroxy group, or a carboxy group. Moreover, the first linker compound has at least one other functional group F₂ which is capable of being reacted with at least one functional group F₃ of the second linker compound. As to functional group F₂, the following functional groups are to be mentioned, among others:
C—C-double bonds or C—C-triple bonds or aromatic C—C-bonds;
the thio group or the hydroxy groups;
alkyl sulfonic acid hydrazide, aryl sulfonic acid hydrazide;
1,2-dioles;
1,2-aminoalcohols;
1,2 amino-thioalcohols;
azides;
the amino group —NH₂ or derivatives of the amino groups comprising the structure unit —NH— such as aminoalkyl groups, aminoaryl group, aminoaralkyl groups, or alkarlyamino groups;
the hydroxylamino group —O—NH₂, or derivatives of the hydroxylamino group comprising the structure unit —O—NH—, such as hydroxylalkylamino groups, hydroxylarylamino groups, hydroxylaralkylamino groups, or hydroxalalkarylamino groups;
alkoxyamino groups, aryloxyamino groups, aralkyloxyamino groups, or alkaryloxyamino groups, each comprising the structure unit —NH—O—;
residues having a carbonyl group, -Q-C(=G)-M, wherein G is O or S, and M is, for example, —
   —OH or —SH;
   an alkoxy group, an aryloxy group, an aralkyloxy group, or an alkaryloxy group;
   an alkylthio group, an arylthio group, an aralkylthio group, or an alkarylthio group;
   an alkylcarbonyloxy group, an arylcarbonyloxy group, an aralkylcarbonyloxy group, an alkarylcarbonyloxy group;
   activated esters such as esters of hydroxyl amines having imid structure such as N-hydroxysuccinimide or having a structure unit O—N where N is part of a heteroaryl compound or, with G=O and Q absent, such as aryloxy compounds with a substituted aryl residue such as pentafluorophenyl, paranitrophenyl or trichlorophenyl;
wherein Q is absent or NH or a heteroatom such as S or O;
   —NH—NH₂, or —NH—NH—;
   —NO₂;
   the nitril group;
   carbonyl groups such as the aldehyde group or the keto group;
   the carboxy group;
   the —N═C═O group or the —N═C═S group;
   vinyl halide groups such as the vinyl iodide or the vinyl bromide group or triflate;
   —C≡C—H;
   —(C═NH₂Cl)—OAlkyl
   groups —(C═O)—CH₂-Hal wherein Hal is Cl, Br, or I;
   —CH═CH—SO₂—;
   a disulfide group comprising the structure —S—S—;
   the group

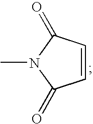

the group

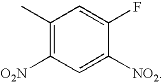

wherein F₃ is a group capable of forming a chemical linkage with one of the above-mentioned groups and is preferably selected from the above-mentioned groups. Moreover, the second linker compound has at least one functional group which is capable of being reacted with the functional group Z of the protein, which is, e.g., an amino group, a thio group, a carboxy group, a reactive carboxy group, an aldehyde group, a keto group, or a hydroxy group. In case one linking compound is used to covalently link the polymer and the protein, the polymer can be reacted with the linking compound and the resulting polymer derivative is reacted with the protein, or the protein can be reacted with the linking compound and the resulting protein derivative is reacted with the polymer. In case two linking compounds L1 and L2 are used, it is possible to react the polymer with L1, react the resulting polymer derivative with L2 and react the resulting polymer derivative with the protein, or to react the protein with L2, react the resulting protein derivative with L1 and react the resulting protein derivative with the polymer. It is also possible to react the polymer with L1 and react the protein with L2 and react the polymer derivative with the protein derivative. Furthermore, it is possible to react L1 with L2, react the resulting compound with the polymer and the resulting polymer derivative with the protein. Furthermore, it is possible to react L1 with L2, react the resulting compound with the protein and the resulting protein derivative with the polymer.

According to an especially preferred embodiment of the present invention, the functional group Z of the protein is an amino group, and the functional group A of the polymer or derivative thereof is a aldehyde group, a keto group or a hemiacetal group. According to a particularly preferred embodiment, functional group Z and functional group A are reacted via a reductive amination reaction.

The reductive amination reaction according to the invention, wherein the polymer or polymer derivative is covalently linked via at least one aldehyde group or keto group or hemiacetal group to at least one amino group of the protein, is preferably carried out at a temperature of from 0 to 40° C., more preferably of from 0 to 25° C. and especially preferably of from 4 to 21° C. The reaction time preferably ranges of from 0.5 to 72 h, more preferably of from 2 to 48 h and especially preferably of from 4 to 7 h. As solvent for the reaction, an aqueous medium is preferred.

Thus, the present invention also relates to a method and a conjugate as described above, wherein the reductive amination is carried out at a temperature of from 4 to 21° C.

Therefore, the present invention also relates to a method and a conjugate as described above, wherein reductive amination is carried out in an aqueous medium.

Thus, the present invention also relates to a method and conjugate as described above, wherein the reductive amination is carried out at a temperature of from 4 to 21° C. in an aqueous medium.

The term “aqueous medium” as used in the context of the present invention relates to a solvent or a mixture of solvents comprising water in the range of from at least 10% per weight, more preferably at least 20% per weight, more preferably at least 30% per weight, more preferably at least 40% per weight, more preferably at least 50% per weight, more preferably at least 60% per weight, more preferably at least 70% per weight, more preferably at least 80% per weight, even more preferably at least 90% per weight or up to 100% per weight, based on the weight of the solvents involved. The preferred reaction medium is water.

The pH value of the reaction medium is generally in the range of from 4 to 9 or from 4 to 8 or from 4 to 7.3.

According to a preferred embodiment of the present invention, the pH at which the reductive amination reaction is carried out, is below 7.3, more preferably smaller or equal 7 and most preferably below 7, i.e. in the acidic range. Preferred ranges are therefore of from 3 to below 7, more preferably of from 3.5 to 6.5, still more preferably of from 4 to 6, still more preferably of from 4.5 to 5.5 and especially preferably about 5.0, i.e. 4.6 or 4.7 or 4.8 or 4.9 or 5.0. or 5.1 or 5.2 or 5.3 or 5.4. Preferred ranges, are among others, 3 to 6.9 or 3 to 6.5 or 3 to 6 or 3 to 5.5 or 3 to 5 or 3 to 4.5 or 3 to 4 or 3 to 3.5 or 3.5 to 6.9 or 3.5 to 6.5 or 3.5 to 6 or 3.5 to 5.5 or 3.5 to 5 or 3.5 to 4.5 or 3.5 to 4 or 4 to 6.9 or 4 to 6.5 or 4 to 6. or 4 to 5.5 or 4 to 5 or 4 to 4.5 or 4.5 to 6.9 or 4.5 to 6.5 or 4.5 to 6 or 4.5 to 5.5 or 4.5 to 5 or 5 to 6.9 or 5 to 6.5 or 5 to 6 or 5 to 5.5 or 5.5 to 6.9 or 5.5 to 6.5 or 5.5 to 6 or 6 to 6.9 or 6 to 6.5 or 6.5 to 6.9.

Therefore, the present invention also relates to a method and a conjugate as described above, wherein the reductive amination is carried out at a pH of 7 or less, more preferably at a pH of 6 or less.

Thus, the present invention also relates to a method and conjugate as described above, wherein the reductive amination is carried out at a temperature of from 4 to 21. ° C. at a pH of 7 or less, preferably of 6 or less.

Hence, the present invention also relates to a method and conjugate as described above, wherein the reductive amination is carried out in an aqueous medium at a pH of 7 or less, preferably of 6 or less.

Accordingly, the present invention also relates to a method and conjugate as described above, wherein the reductive amination is carried out at a temperature of from 4 to 21° C. in an aqueous medium at a pH of 7 or less, preferably of 6 or less.

The molar ratio of polymer derivative:protein used for the reaction is preferably in the range of from 200:1 to 5:1, more preferably of from 100:1 to 10:1 and especially preferably of from 75:1 to 20:1.

It was surprisingly found that it was possible, especially at the preferred pH ranges given above, particularly at a pH below 7 and greater or equal 4, to react the polymer derivative predominantly with the amino group located at the N terminus of the protein. The term “predominantly” as used in the context of the present invention relates to an embodiment where at least 80%, preferably at least 85% of the N-terminal amino groups available are reacted via reductive amination. It is also possible to react at least 90% or at least 95% or at least 96% or at least 97% or at least 98% or at least 99% of the N-terminal amino groups available. Although coupling to amino groups other than the N-terminal amino group could not be ruled out completely, it is believed that coupling via reductive amination according to the present invention at a pH of below 7, preferably below 6, took place essentially selectively at the N-terminal amino group.

Therefore, the present invention also relates to a method and a conjugate as described above, wherein the protein comprises the N-terminal amino group and at least one further amino group, said conjugate comprises the polymer being predominantly coupled to the N-terminal amino group.

According to an especially preferred embodiment, the present invention relates to a method of linking aldehyde or keto or hemiacetal functionalized hydroxyalkyl starch or an aldehyde or keto or hemiacetal functionalized hydroxyalkyl starch derivative predominantly to the N-terminal amino group of a protein, said method comprising subjecting said hydroxyalkyl starch or derivative thereof to a reductive amination reaction, at a pH of 7 or less, preferably at a pH of 6 or less, said reductive amination reaction being carried out preferably in an aqueous medium.

According to the present invention, aldehyde functionalized hydroxyalkyl starch or an aldehyde functionalized hydroxyalkyl starch derivative is preferred.

According to a still further preferred embodiment, the present invention relates to a method of linking aldehyde or keto or hemiacetal functionalized hydroxyethyl starch or an aldehyde or keto or hemiacetal functionalized hydroxyethyl starch derivative selectively to the N-terminal amino group of a protein, said method comprising subjecting said hydroxyalkyl starch or derivative thereof to a reductive amination reaction, at a pH of 7 or less, preferably at a pH of 6 or less, said reductive amination reaction being carried out preferably in an aqueous medium, the hydroxyethyl starch employed preferably being hydroxethyl starch having a mean molecular weight of about 10 kD and a DS of about 0.4 or hydroxethyl starch having a mean molecular weight of about 10 kD and a DS of about 0.7 or hydroxethyl starch having a mean molecular weight of about 12 kD and a DS of about 0.4 or hydroxethyl starch having a mean molecular weight of about 12 kD and a DS of about 0.7 or hydroxethyl starch having a mean molecular weight of about 18 kD and a DS of about 0.4 or hydroxethyl starch having a mean molecular weight of about 18 kD and a DS of about 0.7 or hydroxethyl starch having a mean molecular weight of about 50 kD and a DS of about 0.4 or hydroxethyl starch having a mean molecular weight of about 50 kD and a DS of about 0.7.

The reaction of the polymer derivative and the protein between the aldehyde group or keto group or hemiacetal group and the amino group is a reductive amination wherein a Schiff's base is produced. Subsequently after the reaction, this base may be reduced by at least one reductive agent to give a stable linkage between the polymer derivative and the protein. It is also possible to carry out the reaction in the presence of at least one reductive agent. According to a preferred embodiment, the reductive amination reaction is carried out in the presence of at least one reductive agent.

Preferred reductive agents are sodium borohydride, sodium cyanoborohydride, organic borane complex compounds such as a 4-(dimethylamin)pyridine borane complex, N-ethyldiisopropylamine borane complex, N-ethylmorpholine borane complex, N-methylmorpholine borane complex, N-phenylmorpholine borane complex, lutidine borane complex, triethylamine borane complex, or trimethylamine borane complex. Particularly preferred is sodium cyanoborohydride.

Therefore, the present invention also relates to a method and a conjugate as described above, wherein the reductive amination is carried out in the presence of NaCNBH₃.

Hence, the present invention also relates to a method and conjugate as described above, wherein the reductive amination is carried out in an aqueous medium at a pH of 7 or less, preferably of 6 or less in the presence of reductive agent, preferably NaCNBH₃.

Accordingly, the present invention also relates to a method and conjugate as described above, wherein the reductive amination is carried out at a temperature of from 4 to 21° C. in an aqueous medium at a pH of 7 or less, preferably of 6 or less in the presence of reductive agent, preferably NaCNBH₃.

The molar ratio of polymer derivative:protein used for the reaction is preferably in the range of from 200:1 to 10:1 more preferably of from 100:1 to 10:1 and especially preferably of from 75:1 to 20:1.

Therefore, the present invention also relates to a method of producing a conjugate, said method comprising reacting a polymer or a polymer derivative comprising an aldehyde group in an aqueous medium with an amino group of the protein in the presence of a reductive agent, said reductive agent preferably being NaCNBH₃.

According to the first preferred embodiment of the present invention, according to which the polymer comprises at least two aldehyde groups which are introducing in the polymer by a ring-opening oxidation reaction, the polymer preferably comprises at least one structure according to formula

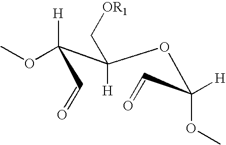

According to this embodiment of the present invention, each oxidation agent or combination of oxidation agents may be employed which is capable of oxidizing at least one saccharide ring of the polymer to give an opened saccharide ring having at least one, preferably at least two aldehyde groups. This reaction is illustrated by the following reaction scheme which shows a saccharide ring of the polymer which is oxidized to give an opened ring having two aldehyde groups:

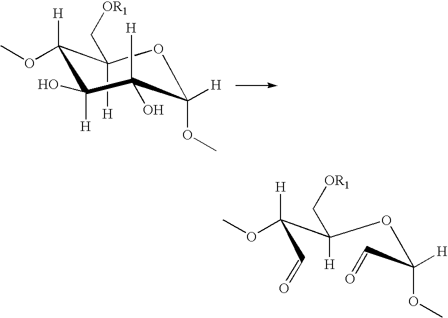

Suitable oxidating agents are, among others, periodates such as alkaline metal periodates or mixtures of two or more thereof, with sodium periodate and potassium periodate being preferred.

Therefore, the present invention also relates to a method and a conjugate as described above, wherein the polymer is subjected to a ring-opening oxidation reaction using a periodate to give a polymer derivative having at least one, preferably at least two aldehyde groups.

For this oxidation reaction, the polymer may be employed with its reducing end either in the oxidized or in the non-oxidized form, the non-oxidized form being preferred.

Therefore, the present invention also relates to a method and a conjugate as described above, wherein the polymer is employed with its reducing end in the non-oxidized form.

The reaction temperature is in a preferred range of from 0 to 40° C., more preferably of from 0 to 25° C. and especially preferably of from 0 to 5° C. The reaction time is in a preferred range of from 1 min to 5 h and especially preferably of from 10 min to 4 h. Depending on the desired degree of oxidation, i.e. the number of aldehyde groups resulting from the oxidation reaction, the molar ratio of periodate polymer may be appropriately chosen.

Therefore, the present invention also relates to a method and a conjugate as described above, wherein the ring-opening oxidation reaction is carried out at a temperature of from 0 to 5° C.

The oxidation reaction of the polymer with periodate is preferably carried out in an aqueous medium, most preferably in water.

Therefore, the present invention also relates to a method and a conjugate as described above, wherein the ring-opening oxidation reaction is carried out in an aqueous medium. The suitable pH value of the reaction mixture may be adjusted by adding at least one suitable buffer. Among the preferred buffers, sodium acetate buffer, phosphate or borate buffers may be mentioned.

The hydroxyethyl starch subjected to said ring-opening oxidation reaction is preferably hydroxethyl starch having a mean molecular weight of about 10 kD and a DS of about 0.4 or hydroxethyl starch having a mean molecular weight of about 10 kD and a DS of about 0.7 or hydroxethyl starch having a mean molecular weight of about 12 kD and a DS of about 0.4 or hydroxethyl starch having a mean molecular weight of about 12 kD and a DS of about 0.7 or hydroxethyl starch having a mean molecular weight of about 18 kD and a DS of about 0.4 or hydroxethyl starch having a mean molecular weight of about 18 kD and a DS of about 0.7 or hydroxethyl starch having a mean molecular weight of about 50 kD and a DS of about 0.4 or hydroxethyl starch having a mean molecular weight of about 50 kD and a DS of about 0.7.

The resulting polymer derivative may be purified from the reaction mixture by at least one suitable method. If necessary, the polymer derivative may be precipitated prior to the isolation by at least one suitable method.

If the polymer derivative is precipitated first, it is possible, e.g., to contact the reaction mixture with at least one solvent or solvent mixture other than the solvent or solvent mixture present in the reaction mixture at suitable temperatures. According to a particularly preferred embodiment of the present invention where an aqueous medium, preferably water is used as solvent, the reaction mixture is contacted with 2-propanol or with am mixture of acetone and ethanol, preferably a 1:1 mixture (v/v), indicating equal volumes of said compounds, at a temperature, preferably in the range of from −20 to +50° C. and especially preferably in the range of from −20 to 25° C.

Isolation of the polymer derivative may be carried out by a suitable process which may comprise one or more steps. According to a preferred embodiment of the present invention, the polymer derivative is first separated off the reaction mixture or the mixture of the reaction mixture with, e.g., aqueous 2-propanol mixture, by a suitable method such as centrifugation or filtration. In a second step, the separated polymer derivative may be subjected to a further treatment such as an after-treatment like dialysis, centrifugal filtration or pressure filtration, ion exchange chromatography, reversed phase chromatography, HPLC, MPLC, gel filtration and/or lyophilisation. According to an even more preferred embodiment, the separated polymer derivative is first dialysed, preferably against water, and then lyophilized until the solvent content of the reaction product is sufficiently low according to the desired specifications of the product. Lyophilisation may be carried out at temperature of from 20 to 35° C., preferably of from 20 to 30° C.

According to a preferred embodiment, the oxidized polymer resulting from the oxidation reaction is purified using at least one suitable method such as ultrafiltration and/or dialysis in order to, e.g., remove undesirable low molecular weight salts and polymer components, thereby also offering a means of controlling the molecular weight range of oxidized polymer.

The oxidized polymer can be used directly for the reaction with the protein or is suitably recovered in a first step, e.g. by lyophilization, and redissolved in water for conjugation to the protein in a second step. As to the coupling of at least one amino group of the protein with at least one aldehyde group of the polymer by reductive amination, reference is made to the detailed disclosure above concerning the specific reaction parameters of the reductive amination reaction such as pH or temperature.

According to the second preferred embodiment, the polymer is reacted with an at least bifunctional compound comprising at least one functional group M capable of being reacted with the polymer and at least one functional group Q which is an aldehyde group or a keto group or a hemiacetal group and which is reacted with an amino group of the protein by reductive amination.

The oxidized polymer can be used directly for the reaction with the protein or is suitably recovered in a first step, e.g. by lyophilization, and redissolved in water for conjugation to the protein in a second step. As to the coupling of at least one amino group of the protein with at least one aldehyde group of the polymer by reductive amination, reference is made to the detailed disclosure above concerning the specific reaction parameters of the reductive amination reaction such as pH or temperature. According to especially preferred embodiments of the present invention, the reductive amination is preferably carried out at a temperature of from 0 to 5° C. such as about 4° C. at a pH of about 4.5 to 5.5 such as about 5.0 and for a reaction time of about 20 to 30 h such as about 24 h.

According to the second preferred embodiment, the polymer is reacted with an at least bifunctional compound comprising at least one functional group M capable of being reacted with the polymer and at least one functional group Q which is an aldehyde group, a keto group or a hemiacetal group and which is reacted with an amino group of the protein by reductive amination.

It is preferred to employ a compound having, apart from the aldehyde group or keto group or hemiacetal group, at least one carboxy group or at least one reactive carboxy group, preferably one carboxy group or one reactive carboxy group. The aldehyde group or keto group or hemiacetal group and the carboxy group or the reactive carboxy group may be separated by any suitable spacer. Among others, the spacer may be an optionally substituted, linear, branched and/or cyclic hydrocarbon residue. Generally, the hydrocarbon residue has from 1 to 60, preferably from 1 to 40, more preferably from 1 to 20, more preferably from 2 to 10, more preferably from 2 to 6 and especially preferably from 2 to 4 carbon atoms. If heteroatoms are present, the separating group comprises generally from 1 to 20, preferably from 1 to 8 and especially preferably from 1 to 4 heteroatoms. The hydrocarbon residue may comprise an optionally branched alkyl chain or an aryl group or a cycloalkyl group having, e.g., from 5 to 7 carbon atoms, or be an aralkyl group, an alkaryl group where the alkyl part may be a linear and/or cyclic alkyl group. According to an even more preferred embodiment, the hydrocarbon residue is an aryl residue having 5 to 7 and preferably 6 carbon atoms. Most preferably, the hydrocarbon residue is the benzene residue. According to this preferred embodiment, the carboxy group and the aldehyde group may be located at the benzene ring in 1,4-position, 1,3-position or 1,2-position, the 1,4-position being preferred.

As reactive carboxy group, a reactive ester, isothiocyanates or isocyanate may be mentioned. Preferred reactive esters are derived from N-hydroxy succinimides such as N-hydroxy succinimide or Sulfo-N-hydroxy succinimide, suitably substituted phenols such as p-nitrophenol, o,p-dinitrophenol, o,o′-dinitrophenol, trichlorophenol such as 2,4,6-trichlorophenol or 2,4,5-trichlorophenol, trifluorophenol such as 2,4,6-trifluorophenol or 2,4,5-trifluorophenol, pentachlorophenol, pentafluorophenol, or hydroxyazoles such as hydroxy benzotriazole. Especially preferred are N-hydroxy succinimides, with N-hydroxy succinimide and Sulfo-N-hydroxy succinimide being especially preferred. All alcohols may be employed alone or as suitable combination of two or more thereof. As reactive ester, pentafluorophenyl ester and N-hydroxy succinimide ester are especially preferred.

Thus, according to a preferred embodiment, the present invention relates to a method and a conjugate as described above, wherein the polymer is reacted with formylbenzoic acid.

According to another preferred embodiment, the present invention relates to a method and a conjugate as described above, wherein the polymer is reacted with formylbenzoic acid pentafluorophenyl ester.

According to yet another preferred embodiment, the present invention relates to a method and a conjugate as described above, wherein the polymer is reacted with formylbenzoic acid N-hydroxysuccinimide ester.

According to yet another embodiment, the present invention relates to a method and a conjugate as described above, wherein the polymer is reacted with 4-(4-formyl-3,5-dimethoxyphenoxy)butyric acid.

The hydroxyethyl starch subjected to the reaction with the compound comprising M, M preferably being a carboxy group or a reactive carboxy group and Q being an aldehyde group or a keto group or a hemiacetal group, is most preferably hydroxethyl starch having a mean molecular weight of about 10 kD and a DS of about 0.7. Also possible are hydroxethyl starches having a mean molecular weight of about 10 kD and a DS of about 0.4 or hydroxethyl starch having a mean molecular weight of about 12 kD and a DS of about 0.4 or hydroxethyl starch having a mean molecular weight of about 12 kD and a DS of about 0.7 or hydroxethyl starch having a mean molecular weight of about 18 kD and a DS of about 0.4 or hydroxethyl starch having a mean molecular weight of about 18 kD and a DS of about 0.7 or hydroxethyl starch having a mean molecular weight of about 50 kD and a DS of about 0.4 or hydroxethyl starch having a mean molecular weight of about 50 kD and a DS of about 0.7. Particularly preferably, the hydroxyalkyl starch and even more preferably the hydroxyethyl starch is employed with its reducing end in the oxidized form.

The resulting polymer derivative with the aldehyde group or the keto group or the hemiacetal group is subsequently reacted with an amino group of the protein via reductive amination. As to the coupling of at least one amino group of the protein with at least one aldehyde group or keto group or hemiacetal group of the polymer by reductive amination, reference is made to the detailed disclosure above concerning the specific reaction parameters of the reductive amination reaction such as pH or temperature. According to an especially preferred embodiment of the present invention, the reaction with the amino group of the protein is preferably carried out at a temperature of from 0 to 40° C., more preferably of from 0 to 25° C. and especially preferably of from 4 to 21° C. The reaction time preferably ranges of from 30 min to 72 h, more preferably of from 2 to 48 h and especially preferably of from 4 h to 17 h. As solvent for the reaction, an aqueous medium is preferred. The pH value of the reaction medium is preferably in the range of from 4 to 9, more preferably of from 4 to 8 and especially preferably of from 4.5 to 5.5.

According to the third preferred embodiment, the polymer is reacted at its optionally oxidized reducing end with an at least bifunctional compound comprising an amino group M and a functional group Q, wherein said amino group M is reacted with the optionally oxidized reducing end of the polymer and wherein the functional group Q is chemically modified to give an aldehyde functionalized polymer derivative which is reacted with an amino group of the protein by reductive amination.

As to functional group Q, the following functional groups are to be mentioned, among others:
C—C-double bonds or C—C-triple bonds or aromatic C—C-bonds;
the thio group or the hydroxy groups;
alkyl sulfonic acid hydrazide, aryl sulfonic acid hydrazide;
1,2-dioles;
1,2 amino-thioalcohols;
azides;
1,2-aminoalcohols;
the amino group —NH₂ or derivatives of the amino groups comprising the structure unit —NH— such as aminoalkyl groups, aminoaryl group, aminoaralkyl groups, or alkarlyamino groups;
the hydroxylamino group —O—NH₂, or derivatives of the hydroxylamino group comprising the structure unit —O—NH—, such as hydroxylalkylamino groups, hydroxylarylamino groups, hydroxylaralkylamino groups, or hydroxalalkarylamino groups;
alkoxyamino groups, aryloxyamino groups, aralkyloxyamino groups, or alkaryloxyamino groups, each comprising the structure unit —NH—O—;
residues having a carbonyl group, -Q-C(=G)-M, wherein G is O or S, and M is, for example,
   —OH or —SH;
   an alkoxy group, an aryloxy group, an aralkyloxy group, or an alkaryloxy group;
   an alkylthio group, an arylthio group, an aralkylthio group, or an alkarylthio group;
   an alkylcarbonyloxy group, an arylcarbonyloxy group, an aralkylcarbonyloxy group, an alkarylcarbonyloxy group;
   activated esters such as esters of hydroxyl amines having imid structure such as N-hydroxysuccinimide or having a structure unit O—N where N is part of a heteroaryl compound or, with G=O and Q absent, such as aryloxy compounds with a substituted aryl residue such as pentafluorophenyl, paranitrophenyl or trichlorophenyl;
wherein Q is absent or NH or a heteroatom such as S or O;
   —NH—NH₂, or —NH—NH—;
   —NO₂;
   the nitril group;
   carbonyl groups such as the aldehyde group or the keto group;
   the carboxy group;
   the —N═C═O group or the —N═C═S group;
   vinyl halide groups such as the vinyl iodide or the vinyl bromide group or triflate;
   —C≡C—H;
   —(C═NH₂Cl)—OAlkyl
   groups —(C═O)—CH₂-Hal wherein Hal is Cl, Br, or I;
   —CH═CH—SO₂—;
   a disulfide group comprising the structure —S—S—;
   the group

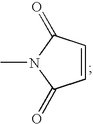

the group

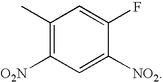

According to a preferred embodiment of the present invention, the term “functional group Q” relates to a functional group Q which comprises the chemical structure —NH—.

According to one preferred embodiment of the present invention, the functional group M is a group having the structure R′—NH— where R′ is hydrogen or a alkyl, cycloalkyl, aryl, aralkyl, arylcycloalkyl, alkaryl or cycloalkylaryl residue where the cycloalkyl, aryl, aralkyl, arylcycloalkyl, alkaryl or cycloalkylaryl residue may be linked directly to the NH group or, according to another embodiment, may be linked by an oxygen bridge to the NH group. The alkyl, cycloalkyl, aryl, aralkyl, arylcycloalkyl, alkaryl, or cycloalkylaryl residues may be suitably substituted. As preferred substituents, halogenes such as F, Cl or Br may be mentioned. Especially preferred residues R′ are hydrogen, alkyl and alkoxy groups, and even more preferred are hydrogen and unsubstituted alkyl and alkoxy groups.

Among the alkyl and alkoxy groups, groups with 1, 2, 3, 4, 5, or 6 C atoms are preferred. More preferred are methyl, ethyl, propyl, isopropyl, methoxy, ethoxy, propoxy, and isopropoxy groups. Especially preferred are methyl, ethyl, methoxy, ethoxy, and particular preference is given to methyl or methoxy.

According to another embodiment of the present invention, the functional group M has the structure R′—NH—R″— where R″ preferably comprises the structure unit —NH— and/or the structure unit —(C=G)- where G is O or S, and/or the structure unit —SO₂—. Specific examples for the functional group R″ are

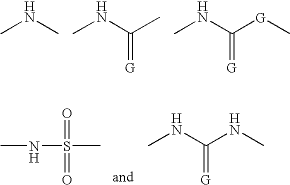

where, if G is present twice, it is independently O or S.

Therefore, the present invention also relates to a method and a conjugate as mentioned above wherein the functional group M is selected from the group consisting of

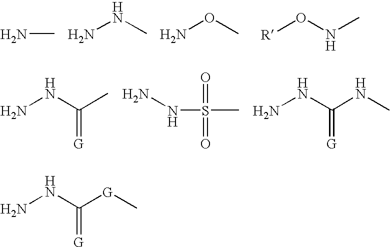

wherein G is O or S and, if present twice, independently O or S, and R′ is methyl.

According to a particularly preferred embodiment of the present invention, the functional group M is an amino group —NH₂.

The term “amino group Q” relates to a functional group Q which comprises the chemical structure —NH—.

According to a preferred embodiment of the present invention, the functional group Q is a group having the structure R′—NH— where R′ is hydrogen or a alkyl, cycloalkyl, aryl, aralkyl, arylcycloalkyl, alkaryl or cycloalkylaryl residue where the cycloalkyl, aryl, aralkyl, arylcycloalkyl, alkaryl or cycloalkylaryl residue may be linked directly to the NH group or, according to another embodiment, may be linked by an oxygen bridge to the NH group. The alkyl, cycloalkyl, aryl, aralkyl, arylcycloalkyl, alkaryl, or cycloalkylaryl residues may be suitably substituted. As preferred substituents, halogenes such as F, Cl or Br may be mentioned. Especially preferred residues R′ are hydrogen, alkyl and alkoxy groups, and even more preferred are hydrogen and unsubstituted alkyl and alkoxy groups.

Among the alkyl and alkoxy groups, groups with 1, 2, 3, 4, 5, or 6 C atoms are preferred. More preferred are methyl, ethyl, propyl, isopropyl, methoxy, ethoxy, propoxy, and isopropoxy groups. Especially preferred are methyl, ethyl, methoxy, ethoxy, and particular preference is given to methyl or methoxy.

According to another embodiment of the present invention, the functional group Q has the structure R′—NH—R″— where R″ preferably comprises the structure unit —NH— and/or the structure unit —(C=G)- where G is O or S, and/or the structure unit —SO₂—. According to more preferred embodiments, the functional group R″ is selected from the group consisting of

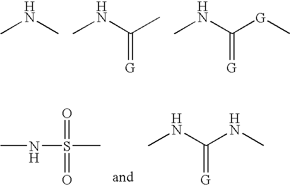

where, if G is present twice, it is independently O or S.

Therefore, the present invention also relates to a method and a conjugate as mentioned above wherein the functional group Q is selected from the group consisting of

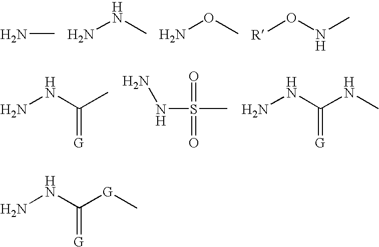

wherein G is O or S and, if present twice, independently O or S, and R′ is methyl.

According to a particularly preferred embodiment of the present invention, the functional group Q is an amino group —NH₂.

According to a still further preferred embodiment of the present invention, both M and Q comprise an amino group —NH—. According to a particularly preferred embodiment, both M and Q are an amino group —NH₂.

According to a preferred embodiment of the present invention, the compound comprising M and Q is a homobifunctional compound, more preferably a homobifunctional compound comprising, as functional groups M and Q, most preferably the amino group —NH₂, or according to other embodiments, the hydroxylamino group —O—NH₂ or the group

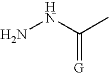

with G preferably being O. Specific examples for these compounds comprising M and Q are

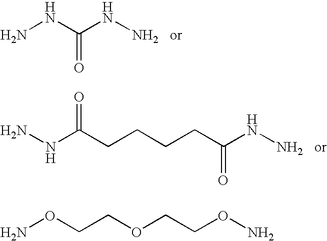

The hydroxyethyl starch subjected to the reaction with the compound comprising M, M preferably being an amino group —NH— and more preferably being an amino group —NH₂, still more preferably both M and Q comprising an amino group —NH— and particularly preferably both M and Q comprising an amino group —NH₂, is preferably hydroxethyl starch having a mean molecular weight of about 10 kD and a DS of about 0.4 or hydroxethyl starch having a mean molecular weight of about 10 kD and a DS of about 0.7. Also possible are or hydroxethyl starches having mean molecular weight of about 12 kD and a DS of about 0.4 or hydroxethyl starch having a mean molecular weight of about 12 kD and a DS of about 0.7 or hydroxethyl starch having a mean molecular weight of about 18 kD and a DS of about 0.4 or hydroxethyl starch having a mean molecular weight of about 18 kD and a DS of about 0.7 or hydroxethyl starch having a mean molecular weight of about 50 kD and a DS of about 0.4 or hydroxethyl starch having a mean molecular weight of about 50 kD and a DS of about 0.7.

In case both M and Q are an amino group —NH₂, M and Q may be separated by any suitable spacer. Among others, the spacer may be an optionally substituted, linear, branched and/or cyclic hydrocarbon residue. Generally, the hydrocarbon residue has from 1 to 60, preferably from 1 to 40, more preferably from 1 to 20, more preferably from 2 to 10, more preferably from 2 to 6 and especially preferably from 2 to 4 carbon atoms. If heteroatoms are present, the separating group comprises generally from 1 to 20, preferably from 1 to 8 and especially preferably from 1 to 4 heteroatoms. The hydrocarbon residue may comprise an optionally branched alkyl chain or an aryl group or a cycloalkyl group having, e.g., from 5 to 7 carbon atoms, or be an aralkyl group, an alkaryl group where the alkyl part may be a linear and/or cyclic alkyl group. According to an even more preferred embodiment, the hydrocarbon residue is an alkyl chain of from 1 to 20, preferably from 2 to 10, more preferably from 2 to 6, and especially preferably from 2 to 4 carbon atoms.

Therefore, the present invention also relates to a method and a conjugate as described above, wherein the polymer is reacted with 1,4-diaminobutane, 1,3-diaminopropane or 1,2-diaminoethane to give a polymer derivative.

The reaction of the at least bifunctional compound comprising M and Q with the polymer is preferably carried out at a temperature of from 0 to 100° C., more preferably of from 4 to 80° C. and especially preferably of from 20 to 80° C.; the reaction time preferably ranges of from 4 h to 7 d, more preferably of from 10 h to 5 d and especially preferably of from 17 to 4 h. The molar ratio of at least bifunctional compound polymer is preferably in the range of from 10 to 200, specially from 50 to 100.

As solvent for the reaction of the at least bifunctional compound with the polymer, at least one aprotic solvent, particularly preferably an anhydrous aprotic solvent having a water content of not more than 0.5 percent by weight, preferably of not more than 0.1 percent by weight is preferred. Suitable solvents are, among others, dimethyl sulfoxide (DMSO), N-methylpyrrolidone, dimethyl acetamide (DMA), dimethyl formamide (DMF) and mixtures of two or more thereof.

As solvent for the reaction of the at least bifunctional compound with the polymer, also an aqueous medium may be used.

According to a preferred embodiment, the polymer derivative comprising the polymer and the at least bifunctional compound is chemically modified at the free functional group Q to give a polymer derivative comprising an aldehyde group or keto group or hemiacetal group. According to this embodiment, it is preferred to react the polymer derivative with at least one at least bifunctional compound which comprises a functional group capable of being reacted with the functional group Q and an aldehyde group or keto group or hemiacetal group.

As at least bifunctional compound, each compound is suitable which has an aldehyde group or keto group or hemiacetal group and at least one functional group which is capable of forming a linkage with the functional group Q of the polymer derivative. The at least one functional group is selected from the same pool of functional groups as Q and is chosen to be able to be reacted with Q. In the preferred case that Q is an amino group —NH₂, it is preferred to employ a compound having, apart from the aldehyde group or keto group or hemiacetal group, at least one carboxy group or at least one reactive carboxy group, preferably one carboxy group or one reactive carboxy group. The aldehyde group group or keto group or hemiacetal group and the carboxy group or the reactive carboxy group may be separated by any suitable spacer. Among others, the spacer may be an optionally substituted, linear, branched and/or cyclic hydrocarbon residue. Generally, the hydrocarbon residue has from 1 to 60, preferably from 1 to 40, more preferably from 1 to 20, more preferably from 2 to 10, more preferably from 2 to 6 and especially preferably from 2 to 4 carbon atoms. If heteroatoms are present, the separating group comprises generally from 1 to 20, preferably from 1 to 8 and especially preferably from 1 to 4 heteroatoms. The hydrocarbon residue may comprise an optionally branched alkyl chain or an aryl group or a cycloalkyl group having, e.g., from 5 to 7 carbon atoms, or be an aralkyl group, an alkaryl group where the alkyl part may be a linear and/or cyclic alkyl group. According to an even more preferred embodiment, the hydrocarbon residue is an aryl residue having 5 to 7 and preferably 6 carbon atoms. Most preferably, the hydrocarbon residue is the benzene residue. According to this preferred embodiment, the carboxy group and the aldehyde group may be located at the benzene ring in 1,4-position, 1,3-position or 1,2-position, the 1,4-position being preferred.

As reactive carboxy group, a reactive ester, isothiocyanates or isocyanate may be mentioned. Preferred reactive esters are derived from N-hydroxy succinimides such as N-hydroxy succinimide or Sulfo-N-hydroxy succinimide, suitably substituted phenols such as p-nitrophenol, o,p-dinitrophenol, o,o′-dinitrophenol, trichlorophenol such as 2,4,6-trichlorophenol or 2,4,5-trichlorophenol, trifluorophenol such as 2,4,6-trifluorophenol or 2,4,5-trifluorophenol, pentachlorophenol, pentafluorophenol, or hydroxyazoles such as hydroxy benzotriazole. Especially preferred are N-hydroxy succinimides, with N-hydroxy succinimide and Sulfo-N-hydroxy succinimide being especially preferred. All alcohols may be employed alone or as suitable combination of two or more thereof. As reactive ester, pentafluorophenyl ester and N-hydroxy succinimide ester are especially preferred.

Thus, according to a preferred embodiment, the present invention relates to a method and a conjugate as described above, wherein the polymer derivative comprising Q, Q being an amino group —NH₂, is further reacted with formylbenzoic acid.

According to another embodiment, the present invention relates to a method and a conjugate as described above, wherein the polymer derivative comprising Q, Q being an amino group, is further reacted with formylbenzoic acid pentafluorophenyl ester.

According to yet another embodiment, the present invention relates to a method and a conjugate as described above, wherein the polymer derivative comprising Q, Q being an amino group, is further reacted with formylbenzoic acid N-hydroxysuccinimide ester.

According to yet another embodiment, the present invention relates to a method and a conjugate as described above, wherein the polymer derivative comprising Q, Q being an amino group, is further reacted with 4-(4-formyl-3,5-dimethoxyphenoxy)butyric acid.

As solvent for the reaction of the polymer derivative comprising an amino group and, e.g., formylbenzoic acid, at least one aprotic solvent, particularly preferably an anhydrous aprotic solvent having a water content of not more than 0.5 percent by weight, preferably of not more than 0.1 percent by weight is preferred. Suitable solvents are, among others, dimethyl sulfoxide (DMSO), N-methylpyrrolidone, dimethyl acetamide (DMA), dimethyl formamide (DMF) and mixtures of two or more thereof.

The reaction is preferably carried out at a temperature of from 0 to 40° C., more preferably of from 0 to 25. ° C. and especially preferably of from 15 to 25° C. for a reaction time preferably of from 0.5 to 24 h and especially preferably of from 1 to 17 h.

According to a preferred embodiment, the reaction is carried out in the presence of an activating agent. Suitable activating agents are, among others, carbodiimides such as diisopropyl carbodimide (DIC), dicyclohexyl carbodiimides (DCC), 1-ethyl-3-(3-dimethylaminopropyl)carbodiimide (EDC), with diisopropyl carbodimide (DIC) being especially preferred.

The resulting polymer derivative may be purified from the reaction mixture by at least one suitable method. If necessary, the polymer derivative may be precipitated prior to the isolation by at least one suitable method.

If the polymer derivative is precipitated first, it is possible, e.g., to contact the reaction mixture with at least one solvent or solvent mixture other than the solvent or solvent mixture present in the reaction mixture at suitable temperatures. According to a particularly preferred embodiment of the present invention where an aqueous medium, preferably water is used as solvent, the reaction mixture is contacted with 2-propanol or with am mixture of acetone and ethanol, preferably a 1:1 mixture (v/v), indicating equal volumes of said compounds, at a temperature, preferably in the range of from −20 to +50° C. and especially preferably in the range of from −20 to 25° C.

Isolation of the polymer derivative may be carried out by a suitable process which may comprise one or more steps. According to a preferred embodiment of the present invention, the polymer derivative is first separated off the reaction mixture or the mixture of the reaction mixture with, e.g., aqueous 2-propanol mixture, by a suitable method such as centrifugation or filtration. In a second step, the separated polymer derivative may be subjected to a further treatment such as an after-treatment like dialysis, centrifugal filtration or pressure filtration, ion exchange chromatography, reversed phase chromatography, HPLC, MPLC, gel filtration and/or lyophilisation. According to an even more preferred embodiment, the separated polymer derivative is first dialysed, preferably against water, and then lyophilized until the solvent content of the reaction product is sufficiently low according to the desired specifications of the product. Lyophilisation may be carried out at temperature of from 20 to 35° C., preferably of from 20 to 30° C.

The resulting polymer derivative with the aldehyde group or keto group or hemiacetal group is subsequently reacted with an amino group of the protein via reductive amination. As to the coupling of at least one amino group of the protein with at least one aldehyde group or keto group or hemiacetal group of the polymer by reductive amination, reference is made to the detailed disclosure above concerning the specific reaction parameters of the reductive amination reaction such as pH or temperature. According to an especially preferred embodiment of the present invention, the reductive amination is carried out at a temperature of from 0 to 10° C. such as from 1 to 8° C. or from 2 to 6° C. such as about 4° C. at a pH of about 4.5 to 5.5 such as about 5.0. The reaction time is about 10 to 20 h such as from 12 to 19 h or from 14 to 18 h such as about 17 h or about 20 to 30 h such as about 24 h.

Thus, according to the above-mentioned preferred embodiments, the present invention also relates, in case the polymer was reacted via its oxidized reducing end, to a conjugate according to the formula

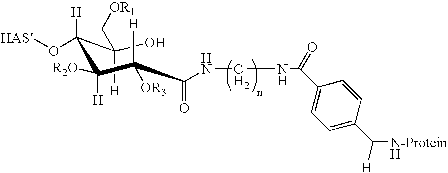

According to an especially preferred embodiment, the polymer is hydroxyethyl starch, i.e. HAS′ is HES′, and n=2, 3, or 4, most preferably 4, as described above. Therefore, in case the polymer was reacted via its oxidized reducing end, the present invention also relates to a conjugate according to the formula

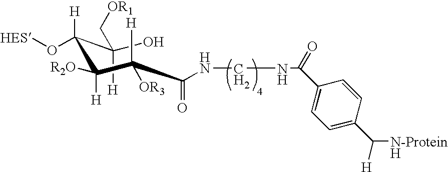

According to another preferred embodiment, the present invention also relates, in case the polymer was reacted via its oxidized reducing end, to a conjugate according to the formula

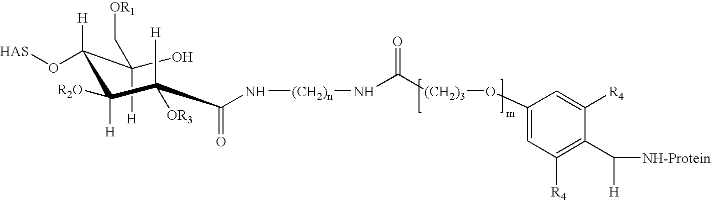

wherein n=2, 3, or 4, R₄ being independently hydrogen or a methoxy group, and m=0 in case R₄ is hydrogen and m=1 in case R₄ is methoxy, HAS preferably being HES′.

In each of the formulae above, the nitrogen attached to the protein derives from the amino group of the protein the polymer derivative is linked to via the aldehyde group.

With respect to the above-mentioned embodiments according to which the functional groups M and Q comprise an amino group —NH₂, it is also possible that M is an amino group —NH₂ and Q comprises a beta hydroxy amino group —CH(OH)—CH₂—NH₂ and preferably is a beta hydroxy amino group.

Therefore, the present invention also relates to a method and a conjugate as described above, wherein the amino group Q of the compound comprising two amino groups M and Q, is a beta hydroxy amino group —CH(OH)—CH₂—NH₂.

In this case, M and Q may be separated by any suitable spacer. Among others, the spacer may be an optionally substituted, linear, branched and/or cyclic hydrocarbon residue. Generally, the hydrocarbon residue has from 1 to 60, preferably from 1 to 40, more preferably from 1 to 20, more preferably from 2 to 10, more preferably from 1 to 6 and especially preferably from 1 to 2 carbon atoms. If heteroatoms are present, the separating group comprises generally from 1 to 20, preferably from 1 to 8 and especially preferably from 1 to 4 heteroatoms. The hydrocarbon residue may comprise an optionally branched alkyl chain or an aryl group or a cycloalkyl group having, e.g., from 5 to 7 carbon atoms, or be an aralkyl group, an alkaryl group where the alkyl part may be a linear and/or cyclic alkyl group. According to an even more preferred embodiment, the hydrocarbon residue is an alkyl chain of from 1 to 20, preferably from 1 to 10, more preferably from 1 to 6, more preferably from 1 to 4 carbon atoms and especially preferably from 1 to 2 carbon atoms. Still more preferably, M and Q are separated by a methylene group.

Therefore, the present invention also relates to a method and a conjugate as described above, wherein the polymer is reacted with 1,3-diamino-2-hydroxypropane.

In case the polymer is reacted via its oxidized reducing end, a polymer derivative according to the formula results

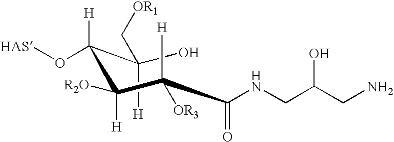

especially preferably with HAS′=HES′

The reaction of the at least bifunctional compound comprising M and Q, particularly preferably 1,3-diamino-2-hydroxypropane, with the polymer is preferably carried out at a temperature of from 40 to 120° C., more preferably of from 40 to 90° C. and especially preferably of from 60 to 80° C. The reaction time preferably ranges from 17 to 168 h, more preferably from 17 to 96 h and especially preferably from 48 to 96 h. The molar ratio of at least bifunctional compound:polymer is preferably in the range of from 200:1 to 10:1, specially from 50:1 to 100:1.

As solvent for the reaction of the at least bifunctional compound with the polymer, at least one aprotic solvent, preferably an anhydrous aprotic solvent having a water content of not more than 0.5 percent by weight, preferably of not more than 0.1 percent by weight is preferred. Suitable solvents are, among others, dimethyl sulfoxide (DMSO), N-methyl pyrrolidone, dimethyl acetamide (DMA), dimethyl formamide (DMF) and mixtures of two or more thereof.

The beta hydroxy amino group Q of the polymer derivative generally may be reacted with an at least bifunctional compound comprising at least one functional group capable of being reacted with Q and further comprising at least one functional group being an aldehyde group or keto group or hemiacetal group or a functional group capable of being modified to give an aldehyde group or keto group or hemiacetal group. According to another embodiment of the present invention, the beta hydroxy amino group is directly chemically modified to give an aldehyde group by chemical oxidation.

This oxidation may be carried with all suitable oxidation agents which are capable of converting the beta hydroxy amino group to an aldehyde group. Preferred oxidation reagents are periodates such as alkaline metal periodates. Especially preferred is sodium periodate which is preferably employed as aqueous solution. This solution has a preferred iodate concentration of from 1 to 50 mM, more preferably from 1 to 25 mM and especially preferably of from 1 to 10 mM. Oxidation is carried out at a temperature of from 0 to 40° C., preferably from 0 to 25° C. and especially preferably from 4 to 20° C.

The resulting polymer derivative may be purified from the reaction mixture by at least one suitable method. If necessary, the polymer derivative may be precipitated prior to the isolation by at least one suitable method.

If the polymer derivative is precipitated first, it is possible, e.g., to contact the reaction mixture with at least one solvent or solvent mixture other than the solvent or solvent mixture present in the reaction mixture at suitable temperatures. According to a particularly preferred embodiment of the present invention where an aqueous medium, preferably water is used as solvent, the reaction mixture is contacted with 2-propanol or with am mixture of acetone and ethanol, preferably a 1:1 mixture (v/v), indicating equal volumes of said compounds, at a temperature, preferably in the range of from −20 to +50° C. and especially preferably in the range of from −20 to 25° C.

Isolation of the polymer derivative may be carried out by a suitable process which may comprise one or more steps. According to a preferred embodiment of the present invention, the polymer derivative is first separated off the reaction mixture or the mixture of the reaction mixture with, e.g., aqueous 2-propanol mixture, by a suitable method such as centrifugation or filtration. In a second step, the separated polymer derivative may be subjected to a further treatment such as an after-treatment like dialysis, centrifugal filtration or pressure filtration, ion exchange chromatography, reversed phase chromatography, HPLC, MPLC, gel filtration and/or lyophilisation. According to an even more preferred embodiment, the separated polymer derivative is first dialysed, preferably against water, and then lyophilized until the solvent content of the reaction product is sufficiently low according to the desired specifications of the product. Lyophilisation may be carried out at temperature of from 20 to 35° C., preferably of from 20 to 30° C.

Therefore, the present invention also relates to a method and a conjugate as described above, wherein the oxidation of the beta hydroxy amino group Q is carried out using a periodate.

Therefore, the present invention also relates to a method of producing a conjugate, wherein, in case the polymer was employed with oxidized reducing end, a polymer derivative having a beta hydroxy amino group, especially preferably

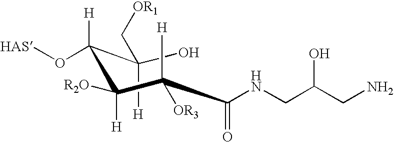

and particularly with HAS′=HES′, is oxidized, preferably with a periodate, to a polymer derivative having an aldehyde group, especially preferably

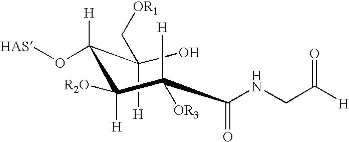

and particularly with HAS′=HES′.

The resulting polymer derivative with the aldehyde group A is subsequently reacted with the protein. Therefore, the present invention also relates to a method of producing a conjugate, said method comprising reacting a polymer derivative having a beta hydroxy amino group, in case the polymer was employed with oxidized reducing end especially preferably according to the formula

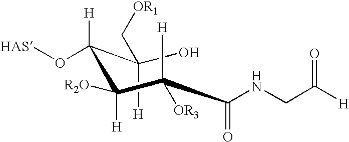

and particularly with HAS′=HES′, with an amino group of the protein.

The resulting polymer derivative with the aldehyde group is subsequently reacted with an amino group of the protein via reductive amination. As to the coupling of at least one amino group of the protein with at least one aldehyde group of the polymer by reductive amination, reference is made to the detailed disclosure above.

Thus, according to the above-mentioned preferred embodiment, the present invention also relates to a conjugate according to the formula

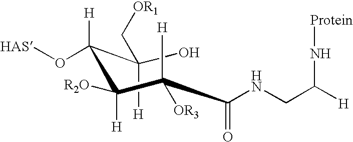

particularly with HAS′=HES′, in case the polymer was employed with oxidized reducing end. In the formula above, the nitrogen attached to the protein derives from the amino group of the protein the polymer derivative is linked to via the aldehyde group.

According to a further embodiment of the present invention, the polymer is first reacted with a suitable compound to give a first polymer derivative comprising at least one reactive carboxy group. This first polymer derivative is then reacted with a further, at least bifunctional compound wherein at least one functional group of this further compound is reacted with at least one reactive carboxy group of the polymer derivative and at least one other functional group of the further compound is an aldehyde group or keto group or hemiacetal group or is a functional group which is chemically modified to give an aldehyde group or keto group or hemiacetal group, and wherein the resulting polymer derivative comprising said aldehyde group or keto group or hemiacetal group is reacted via reductive amination, as described above, with at least one amino group of the protein. It is also possible to alter the sequence of reacting the respective compounds with each other.

According to a first alternative of said further embodiment, the polymer comprising at least one reactive carboxy group is prepared by selectively oxidizing the polymer at its reducing end and subsequently reacting the oxidized polymer being a lactone

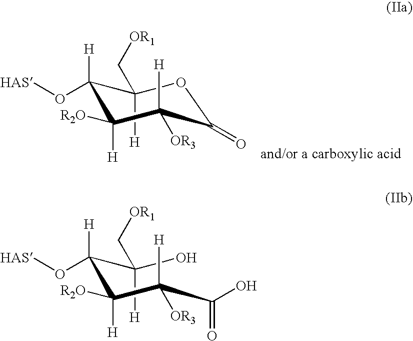

or a suitable salt of the carboxylic acid such as alkali metal salt, preferably as sodium and/or potassium salt, and HAS′ preferably being HES′, with a suitable compound to give the polymer comprising at least one reactive carboxy group.

Oxidation of the polymer, preferably hydroxyethyl starch, may be carried out according to each method or combination of methods which result in compounds having the above-mentioned structures (IIa) and/or (IIb).

Although the oxidation may be carried out according to all suitable method or methods resulting in the oxidized reducing end of hydroxyalkyl starch, it is preferably carried out using an alkaline iodine solution as described, e.g., in DE 196 28 705 A1 the respective contents of which (example A, column 9, lines 6 to 24) is incorporated herein by reference.

Introducing the reactive carboxy group into the polymer which is selectively oxidized at its reducing end may carried out by all conceivable methods and all suitable compounds.

According to a specific method of the present invention, the polymer which is selectively oxidized at its reducing end is reacted at the oxidized reducing end with at least one alcohol, preferably with at least one acidic alcohol such as acidic alcohols having a pK_{A} value in the range of from 6 to 12 or of from 7 to 11 at 25° C. The molecular weight of the acidic alcohol may be in the range of from 80 to 500 g/mole, such as of from 90 to 300 g/mole or of from 100 to 200 g/mole.

Suitable acidic alcohols are all alcohols H—O—R_{A} having an acidic proton and are capable of being with reacted with the oxidized polymer to give the respective reactive polymer ester, preferably according to the formula

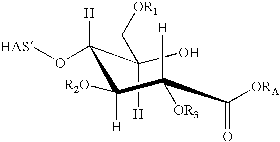

still more preferably according to formula

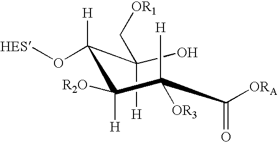

Preferred alcohols are N-hydroxy succinimides such as N-hydroxy succinimide or Sulfo-N-hydroxy succinimide, suitably substituted phenols such as p-nitrophenol, o,p-dinitrophenol, o,o′-dinitrophenol, trichlorophenol such as 2,4,6-trichlorophenol or 2,4,5-trichlorophenol, trifluorophenol such as 2,4,6-trifluorophenol or 2,4,5-trifluorophenol, pentachlorophenol, pentafluorophenol, or hydroxyazoles such as hydroxy benzotriazole. Especially preferred are N-hydroxy succinimides, with N-hydroxysuccinimide and Sulfo-N-hydroxysuccinimide being especially preferred. All alcohols may be employed alone or as suitable combination of two or more thereof. In the context of the present invention, it is also possible to employ a compound which releases the respective alcohol, e.g. by adding diesters of carbonic acid.

Therefore, the present invention also relates to a method as described above, wherein the polymer which is selectively oxidised at its reducing end is activated by reacting the oxidised polymer with an acidic alcohol, preferably with N-hydroxy succinimide and/or Sulfo-N-hydroxy succinimide.

According to a preferred embodiment of the present invention, the polymer which is selectively oxidized at its reducing end is reacted at the oxidized reducing end with at least one carbonic diester R_{B}—O—(C═O)—O—R_{C}, wherein R_{B} and R_{C} may be the same or different. Preferably, this method gives reactive polymers according to the formula

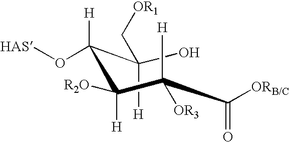

wherein HAS′ is preferably HES′.

As suitable carbonic diester compounds, compounds may be employed whose alcohol components are independently N-hydroxy succinimides such as N-hydroxy succinimide or Sulfo-N-hydroxy succinimide, suitably substituted phenols such as p-nitrophenol, o,p-dinitrophenol, o,o′-dinitrophenol, trichlorophenol such as 2,4,6-trichlorophenol or 2,4,5-trichlorophenol, trifluorophenol such as 2,4,6-trifluorophenol or 2,4,5-trifluorophenol, pentachlorophenol, pentafluorophenol, or hydroxyazoles such as hydroxy benzotriazole. Especially preferred are N,N′-disuccinimidyl carbonate and Sulfo-N,N′-disuccinimidyl carbonate, with N,N′-disuccinimidyl carbonate being especially preferred.

Therefore, the present invention also relates a method as described above, wherein the polymer which is selectively oxidised at its reducing end is activated by reacting the oxidised polymer with N,N′-disuccinimidyl carbonate.

The acidic alcohol is reacted with the oxidized polymer or the salt of the oxidized polymer at a molar ratio of acidic alcohol:polymer preferably of from 5:1 to 50:1, more preferably of from 8:1 to 20:1, at a preferred reaction temperature of from 2 to 40° C., more preferably of from 10 to 30° C. and especially preferably of from 15 to 25° C. The reaction time is preferably in the range of from 1 to 10 h, more preferably of from 2 to 5 h, more preferably of from 2 to 4 h and particularly of from 2 to 3 h.

The carbonic diester compound is reacted with the oxidized polymer or the salt of the oxidized polymer at a molar ratio of diester compound:polymer generally of from 1:1 to 3:1, such as of from 1:1 to 1.5:1. The reaction time is generally in the range of from 0.1 to 12 h, like of from 0.2 to 6 h, or of from 0.5 to 2 h or of from 0.75 to 1.25 h.

According to a preferred embodiment of the present invention, reacting the oxidized polymer with acidic alcohol and/or carbonic diester is carried out in at least one aprotic solvent, such as in an anhydrous aprotic solvent having a water content of not more than 0.5 percent by weight, preferably of not more than 0.1 percent by weight. Suitable solvents are, among others, dimethyl sulfoxide (DMSO), N-methylpyrrolidone, dimethyl acetamide (DMA), dimethyl formamide (DMF) and mixtures of two or more thereof. The reaction temperatures are preferably in the range of from 2 to 40° C., more preferably of from 10 to 30° C.

For reacting the oxidized polymer with the at least one acidic alcohol, at least one additional activating agent is employed.

Suitable activating agents are, among others, carbonyldiimidazole, carbodiimides such as diisopropyl carbodimide (DIC), dicyclohexyl carbodiimides (DCC), 1-ethyl-3-(3-dimethylaminopropyl) carbodiimide (EDC), with dicyclohexyl carbodiimides (DCC) and 1-ethyl-3-(3-dimethylaminopropyl)carbodiimide (EDC) being especially preferred.

Therefore, the present invention also relates to the method as described above, where the polymer which is oxidized at its reducing end, is reacted with an acidic alcohol in the presence of an additional activating agent to give the reactive polymer ester.

According to one embodiment of the present invention, the reaction of the oxidized polymer with carbonic diester and/or acidic alcohol is carried out at a low base activity which may be determined by adding the reaction mixture to water with a volume ratio of water to reaction mixture of 10:1. Prior to the addition, the water which comprises essentially no buffer, has a pH value of 7 at 25° C. After the addition of the reaction mixture and by measuring the pH value, the base activity of the reaction mixture is obtained, having a value of preferably not more than 9.0, more preferably of nor more than 8.0 and especially preferably of not more than 7.5.

According to another embodiment of the present invention, the oxidized polymer is reacted with N-hydroxy succinimide in dry DMA in the absence of water with EDC to selectively give the polymer N-hydroxy succinimide ester according to the formula

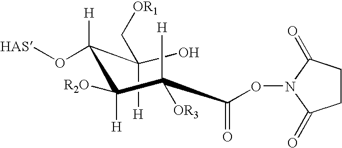

more preferably with HAS′ being HES′.

Surprisingly, this reaction does not give by-products resulting from reactions of EDC with OH groups of HES, and the rearrangement reaction of the O-acyl isourea formed by EDC and the oxidized polymer to the respective N-acyl urea is surprisingly suppressed.

According to another preferred embodiment of the present invention, the oxidized polymer is reacted with N,N′-disuccinimidyl carbonate in dry DMF in the absence of water and in the absence of an activating agent to selectively give the polymer N-hydroxy succinimide ester according to the formula

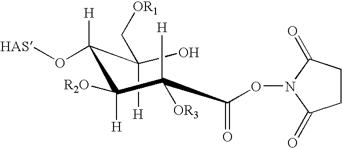

more preferably with HAS′ being HES′.

According to another embodiment of the present invention, the polymer which is selectively oxidized at its reducing end is reacted at the oxidized reducing end with an azolide such as carbonyldiimidazole or carbonyl dibenzimidazole to give a polymer having a reactive carboxy group. In the case of carbonyldiimidazole, a reactive imidazolide polymer derivative according to formula

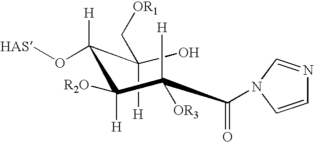

results, wherein HAS′ is preferably HES′.

According to a second alternative of said further embodiment of the present invention regarding the introduction of at least one reactive carboxy group into the polymer, the reactive carboxy group is introduced into the polymer whose reducing end is not oxidized, by reacting at least one hydroxy group of the polymer with a carbonic diester.

Therefore, the present invention also relates to a method and conjugates wherein the reactive carboxy group is introduced in the polymer whose reducing end is not oxidized, by reacting at least one hydroxy group of the polymer with at least one carbonic diester carbonic diester R_{B}—O—(C═O)—O—R_{C}, wherein R_{B} and R_{C} may be the same or different.

According to another embodiment of the present invention, the polymer whose reducing end is not oxidized, is reacted at least one hydroxy group with an azolide such as carbonyldiimidazole, carbonyl-di-(1,2,4-triazole) or carbonyl dibenzimidazol to give a polymer having a reactive carboxy group.

As suitable carbonic diester compounds, compounds may be employed whose alcohol components are independently N-hydroxy succinimides such as N-hydroxy succinimide or Sulfo-N-hydroxy succinimide, suitably substituted phenols such as p-nitrophenol, o,p-dinitrophenol, o,o′-dinitrophenol, trichlorophenol such as 2,4,6-trichlorophenol or 2,4,5-trichlorophenol, trifluorophenol such as 2,4,6-trifluorophenol or 2,4,5-trifluorophenol, pentachlorophenol, pentafluorophenol, or hydroxyazoles such as hydroxy benzotriazole.

Especially preferred are symmetrical carbonic diester compounds, R_{B} and R_{C} thus being the same. The alcohol component of the carbonic diester is preferably selected from the group consisting of N-hydroxy succinimide, sulfonated N-hydroxy succinimide, N-hydroxy benzotriazole, and nitro- and halogen-substituted phenols. Among others, nitrophenol, dinitrophenol, trichlorophenol, trifluorophenol, pentachlorophenol, and pentafluorophenol are preferred. Especially preferred are N,N′-disuccinimidyl carbonate and Sulfo-N,N′-disuccinimidyl carbonate, with N,N′-disuccinimidyl carbonate being especially preferred.

Therefore, the present invention also relates to a hydroxyalkyl starch derivative, preferably a hydroxyethyl starch derivative, wherein at least one hydroxy group, preferably at least two hydroxy groups of said starch have been reacted with a carbonic diester compound to give the respective reactive ester.

According to one embodiment of the present invention, the reaction of the polymer whose reducing end is not oxidized, with the at least one carbonic diester compound is carried out at a temperature of from 2 to 40° C., more preferably of from 10 to 30° C. and especially of from 15 to 25° C. A preferred reaction time ranges from 0.5 to 5 h, more preferably from 1 to 3 h, and especially preferably from 2 to 3 h.

The molar ratio of carbonic diester compound:polymer depends on the degree of substitution of the polymer regarding the number of hydroxy groups reacted with carbonic diester compound relative to the number of hydroxy groups present in the non-reacted polymer.

According to one embodiment of the present invention, the molar ratio of carbonic diester compound:anhydroglucose units of the polymer is in the range of from 1:2 to 1:1000, more preferably of from 1:3 to 1:100 and especially preferably of from 1:10 to 1:50, to give a degree of substitution in the range of from 0.5 to 0.001, preferably of from 0.33 to 0.01 and especially preferably of from 0.1 to 0.02

According to one embodiment of the present invention, reacting the polymer whose reducing end is not oxidized, with carbonic diester is carried out in at least one aprotic solvent, particularly preferably in an anhydrous aprotic solvent having a water content of not more than 0.5 percent by weight, preferably of not more than 0.1 percent by weight. Suitable solvents are, among others, dimethyl sulfoxide (DMSO), N-methylpyrrolidone, dimethyl acetamide (DMA), dimethyl formamide (DMF) and mixtures of two or more thereof.

Therefore, the present invention also relates to a method as described above wherein the reaction of the at least one hydroxy group of the polymer whose reducing end is not oxidised, with the carbonic diester to give a reactive carboxy group is carried out in an anhydrous aprotic polar solvent, the solvent preferably being dimethyl acetamide, dimethyl formamide or a mixture thereof.

The reactive polymer derivative comprising at least one reactive carboxy group, preferably resulting from the reaction of the polymer with the acidic alcohol, the carbonate and/or the azolide, as described above, is further reacted with a further, at least bifunctional compound wherein at least one functional group F₁ of this further compound is reacted with at least one reactive carboxy group of the polymer derivative. As at least one functional group F₁ of the further compound no specific limitations exist given that a reaction with the at least one reactive carboxy group of the polymer is possible. Preferred functional groups F₁ are, e.g., an amino group or a hydroxy group or a thio group or a carboxy group.

The further, at least bifunctional compound comprises at least one other functional group F₂ being an aldehyde group or a functional group F₂ being capable of being chemically modified to give an aldehyde group. The chemical modification may be, e.g., a reaction of the functional group F₂ with a functional group F₃ a further linker compound or an oxidation or a reduction of a suitable functional group F₂.

In case F₂ is reacted with a functional group F₃ of a further compound, the functional group F₂ may be selected from, among others,
C—C-double bonds or C—C-triple bonds or aromatic C—C-bonds;
the thio group or the hydroxy group;
alkyl sulfonic acid hydrazide, aryl sulfonic acid hydrazide;
1,2-dioles;
1,2-aminoalcohols;
1,2 amino-thioalcohols;
azides;
the amino group —NH₂ or derivatives of the amino groups comprising the structure unit —NH— such as aminoalkyl groups, aminoaryl group, aminoaralkyl groups, or alkarlyamino groups;
the hydroxylamino group —O—NH₂, or derivatives of the hydroxylamino group comprising the structure unit —O—NH—, such as hydroxylalkylamino groups, hydroxylarylamino groups, hydroxylaralkylamino groups, or hydroxalalkarylamino groups;
alkoxyamino groups, aryloxyamino groups, aralkyloxyamino groups, or alkaryloxyamino groups, each comprising the structure unit —NH—O—;
residues having a carbonyl group, -Q-C(=G)-M, wherein G is O or S, and M is, for example,
   —OH or —SH;
   an alkoxy group, an aryloxy group, an aralkyloxy group, or an alkaryloxy group;
   an alkylthio group, an arylthio group, an aralkylthio group, or an alkarylthio group;
   an alkylcarbonyloxy group, an arylcarbonyloxy group, an aralkylcarbonyloxy group, an alkarylcarbonyloxy group;
   activated esters such as esters of hydroxyl amines having imid structure such as N-hydroxysuccinimide or having a structure unit O—N where N is part of a heteroaryl compound or, with G=O and Q absent, such as aryloxy compounds with a substituted aryl residue such as pentafluorophenyl, paranitrophenyl or trichlorophenyl;
wherein Q is absent or NH or a heteroatom such as S or O;
   —NH—NH₂, or —NH—NH—;
   —NO₂;
   the nitril group;
   carbonyl groups such as the aldehyde group or the keto group;
   the carboxy group;
   the —N═C═O group or the —N═C═S group;
   vinyl halide groups such as the vinyl iodide or the vinyl bromide group or triflate;
   —C≡C—H;
   —(C═NH₂Cl)—OAlkyl
   groups —(C═O)—CH₂-Hal wherein Hal is Cl, Br, or I;
   —CH═CH—SO₂—;
   a disulfide group comprising the structure —S—S—;
   the group

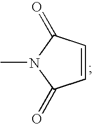

the group

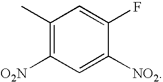

wherein F₃ is a group capable of forming a chemical linkage with one of the above-mentioned groups and is preferably selected from the above-mentioned groups. Moreover, the second linker compound preferably has at least one aldehyde group or keto group or hemiacetal group which is capable of being reacted with an amino group of the protein via reductive amination.

The functional group F₁ and the aldehyde group or keto group or hemiacetal group of the at least bifunctional linking compound which is reacted with the polymer, and/or the functional groups F₁ and F₂ of the at least bifunctional linking compound which is reacted with the polymer, and/or the functional group F₃ and the aldehyde group or keto group or hemiacetal group of the further, at least bifunctional linking compound, may be independently separated by any suitable spacer. Among others, the spacer may be an optionally substituted, linear, branched and/or cyclic, aliphatic and/or aromatic hydrocarbon residue. Generally, the hydrocarbon residue has up to 60, preferably up to 40, more preferably up to 20, more preferably up to 10 carbon atoms. If heteroatoms are present, the separating group comprises generally from 1 to 20, preferably from 1 to 8, more preferably 1 to 6, more preferably 1 to 4 and especially preferably from 1 to 2 heteroatoms. As heteroatom, O is preferred. The hydrocarbon residue may comprise an optionally branched alkyl chain or an aryl group or a cycloalkyl group having, e.g., from 5 to 7 carbon atoms, or be an aralkyl group, an alkaryl group where the alkyl part may be a linear and/or cyclic alkyl group.

Examples of a compound with functional groups F₁ and F₂ are, e.g., optionally substituted diaminoalkane having from 2 to 20 carbon atoms, especially preferably 1,2-diaminoethane, 1,3-diaminopropane, and 1,4-diaminobutane. Preferred examples of a compound with functional groups F₃ and an aldehyde group or a keto group or a hemiacetal group are, e.g., formylbenzoic acid, 4-formylbenzoic acid pentafluorophenyl ester, 4-formylbenzoic acid-N-hydroxysuccinimide ester and 4-(4-formyl-3,5-dimethoxyphenoxy)butyric acid.

Therefore, the present invention also relates to a method of producing a conjugate, said method comprising reacting the polymer, preferably hydroxyethyl starch, at its optionally oxidized reducing end with a compound, selected from the group consisting of acidic alcohols, carbonic diesters and azolides, to give a polymer derivative comprising at least one reactive carboxy group, reacting said polymer derivative with at least one at least bifunctional compound to give a polymer derivative comprising an aldehyde group or a keto group or a hemiacetal group or a functional group capable of being chemically modified to give an aldehyde group or a keto group or a hemiacetal group, optionally chemically modifying said functional group to give a polymer derivative comprising an aldehyde group or a keto group or a hemiacetal group, and reacting the polymer derivative comprising an aldehyde group or a keto group or a hemiacetal group with an amino group of a protein via reductive amination.

Accordingly, the present invention also relates to a conjugate comprising a polymer, preferably hydroxyethyl starch, and a protein covalently linked to each other, obtainable by a method of producing a conjugate, said method comprising reacting the polymer, at its optionally oxidized reducing end with a compound, selected from the group consisting of acidic alcohols, carbonic diesters and azolides, to give a polymer derivative comprising at least one reactive carboxy group, reacting said polymer derivative with at least one at least bifunctional compound to give a polymer derivative comprising an aldehyde group or a keto group or a hemiacetal group or a functional group capable of being chemically modified to give an aldehyde group or a keto group or a hemiacetal group, optionally chemically modifying said functional group to give a polymer derivative comprising an aldehyde group or a keto group or a hemiacetal group, and reacting the polymer derivative comprising an aldehyde group or a keto group or a hemiacetal group with an amino group of a protein via reductive amination.

A specific example of a compound having a functional group F₁ and a functional group F₂ which is oxidized to give an aldehyde group is, e.g., a compound having an amino group as F₁ and a beta hydroxy amino group as F₂. An especially preferred example is 1,3-diamino-2-hydroxypropane. This oxidation may be carried with all suitable oxidation agents which are capable of converting the beta hydroxy amino group to an aldehyde group. Preferred oxidation reagents are periodates such as alkaline metal periodates. Especially preferred is sodium periodate which is preferably employed as aqueous solution. This solution has a preferred iodate concentration of from 1 to 50 mM, more preferably from 1 to 25 mM and especially preferably of from 1 to 10 mM. Oxidation is carried out at a temperature of from 0 to 40° C., preferably from 0 to 25° C. and especially preferably from 4 to 20° C.

The resulting polymer derivative may be purified from the reaction mixture by at least one suitable method. If necessary, the polymer derivative may be precipitated prior to the isolation by at least one suitable method.

If the polymer derivative is precipitated first, it is possible, e.g., to contact the reaction mixture with at least one solvent or solvent mixture other than the solvent or solvent mixture present in the reaction mixture at suitable temperatures. According to a particularly preferred embodiment of the present invention where an aqueous medium, preferably water is used as solvent, the reaction mixture is contacted with 2-propanol or with am mixture of acetone and ethanol, preferably a 1:1 mixture (v/v), indicating equal volumes of said compounds, at a temperature, preferably in the range of from −20 to +50° C. and especially preferably in the range of from −20 to 25° C.

Isolation of the polymer derivative may be carried out by a suitable process which may comprise one or more steps. According to a preferred embodiment of the present invention, the polymer derivative is first separated off the reaction mixture or the mixture of the reaction mixture with, e.g., aqueous 2-propanol mixture, by a suitable method such as centrifugation or filtration. In a second step, the separated polymer derivative may be subjected to a further treatment such as an after-treatment like dialysis, centrifugal filtration or pressure filtration, ion exchange chromatography, reversed phase chromatography, HPLC, MPLC, gel filtration and/or lyophilisation. According to an even more preferred embodiment, the separated polymer derivative is first dialysed, preferably against water, and then lyophilized until the solvent content of the reaction product is sufficiently low according to the desired specifications of the product. Lyophilisation may be carried out at temperature of from 20 to 35° C., preferably of from 20 to 30° C.

According to another preferred embodiment of the present invention, the functional group Z of the protein to be reacted with functional group A of the polymer or polymer derivative is a thiol group.

The thiol group may be present in the protein as such. Moreover, it is possible to introduce a thiol group into the protein according to a suitable method. Among others, chemical methods may be mentioned. If a disulfide bridge is present in the protein, it is possible to reduce the —S—S— structure to get a thiol group. It is also possible to transform an amino group present in the polypeptide into a SH group by reaction the polypeptide via the amino group with a compound which has at least two different functional groups, one of which is capable of being reacted with the amino group and the other is an SH group or a precursor of an SH group e.g. N-succinimidyl-S-acetylthioacetate, N-succinimidyl-S-acetylthiopropionate or N-succinimidyl-3-(pyridyldithio)propionate. It is also possible to introduce an SH group by mutation of the protein such as by introducing an additional cysteine into the protein, exchanging an amino acid to a cysteine or such as removing a cystein from the protein so as to disable another cysteine in the protein to form a disulfide bridge. Most preferably, the polymer is linked to a free cysteine of the protein, especially preferably Cys 17 or Cys 18, wherein Cys 17 is, e.g., present in Granocyte®, and Cys 18 is, e.g., present in Neupogen®.

According to a first embodiment, the functional group Z of the protein is a thiol group and functional group A of the polymer is a halogenacetyl group and wherein A is introduced by reacting the polymer at its optionally oxidized reducing end with an at least bifunctional compound having at least two functional groups each comprising an amino group to give a polymer derivative having at least one functional group comprising an amino group and reacting the polymer derivative with a monohalogen-substituted acetic acid and/or a reactive monohalogen-substituted acetic acid derivative.

As to the at least bifunctional compound having at least two functional groups each comprising an amino group, all compounds are conceivable which are capable of being reacted with the polymer at its optionally reducing end to give a polymer derivative comprising an amino group which can be reacted with a monohalogen-substituted acetic acid and/or a reactive monohalogen-substituted acetic acid derivative.

According to a preferred embodiment, one functional group of the at least bifunctional compound, said functional group being reacted with the optionally oxidized reducing end of the polymer, is selected from the group consisting of

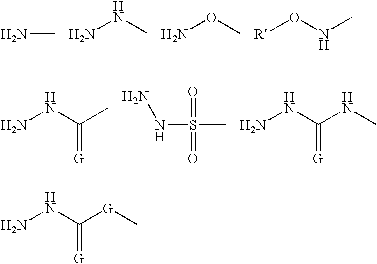

wherein G is O or S and, if present twice, independently O or S, and R′ is methyl.

According to an especially preferred embodiment of the present invention, the functional group of the at least bifunctional compound, said functional group being reacted with the optionally oxidized reducing end, is the amino group —NH₂. According to a still further preferred embodiment, this functional group, most preferably the amino group, is reacted with the oxidized reducing end of the polymer.

According to a preferred embodiment of the present invention, the functional group of the at least bifunctional compound, said functional group being reacted with the monohalogen-substituted acetic acid and/or a reactive monohalogen-substituted acetic acid derivative, is an amino group —NH₂.

The functional groups, preferably both being an amino group —NH₂, of the at least bifunctional compound, said functional groups being reacted with the polymer at its optionally oxidized reducing end, preferably the oxidized reducing end, and the monohalogen-substituted acetic acid and/or a reactive monohalogen-substituted acetic acid derivative, may be separated by any suitable spacer. Among others, the spacer may be an optionally substituted, linear, branched and/or cyclic hydrocarbon residue. Suitable substituents are, among others, alkyl, aryl, aralkyl, alkaryl, halogen, carbonyl, acyl, carboxy, carboxyester, hydroxy, thio, alkoxy and/or alkylthio groups. Generally, the hydrocarbon residue has from 1 to 60, preferably from 1 to 40, more preferably from 1 to 20, more preferably from 2 to 10, more preferably from 2 to 6 and especially preferably from 2 to 4 carbon atoms. If heteroatoms are present, the separating group comprises generally from 1 to 20, preferably from 1 to 8 and especially preferably from 1 to 4 heteroatoms. The hydrocarbon residue may comprise an optionally branched alkyl chain or an aryl group or a cycloalkyl group having, e.g., from 5 to 7 carbon atoms, or be an aralkyl group, an alkaryl group where the alkyl part may be a linear and/or cyclic alkyl group. According to an even more preferred embodiment, the hydrocarbon residue is an alkyl chain of from 1 to 20, preferably from 2 to 10, and especially preferably from 2 to 8 carbon atoms. Thus, preferred at least bifunctional compounds are bifunctional amino compounds, especially preferably 1,8-diamino octane, 1,7-diamino heptane, 1,6-diamino hexane, 1,5-diamino pentane, 1,4-diamino butane, 1,3-diamino propane, and 1,2-diamino ethane. According to a further preferred embodiment, the at least bifunctional compound is a diaminopolyethylenglycol, preferably a diaminopolyethylenglycol according to formula

H₂N—(CH₂—CH₂—O)ₘ—CH₂—CH₂—NH₂

wherein m is an integer, m preferably being 1, 2, 3, or 4.

Therefore, the present invention also relates to a method and a conjugate as described above, wherein the polymer is reacted with 1,8-diaminooctane, 1,7-diaminoheptane, 1,6-diaminohexane, 1,5-diaminopentane, 1,4-diaminobutane, 1,3-diaminopropane, and 1,2-diaminoethane at its oxidized reducing end with to give a polymer derivative according to the formula

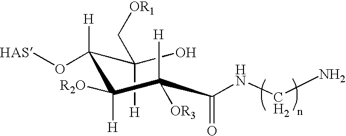

with n=2, 3, 4, 5, 6, 7, or 8, and the polymer especially preferably being HES.

Therefore, the present invention also relates to a method and a conjugate as described above, wherein the polymer is reacted with H₂N—(CH₂—CH₂—O)ₘ—CH₂—CH₂—NH₂ at its oxidized reducing end, wherein m is 1, 2, 3, or 4, to give a polymer derivative according to the formula

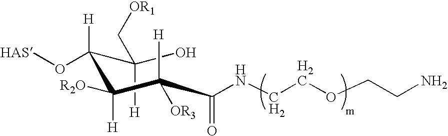

with m=1, 2, 3, or 4, and the polymer especially preferably being HES.

The oxidation of the reducing end of the polymer, preferably hydroxyethyl starch, may be carried out according to each method or combination of methods which result in compounds having the structures (IIa) and/or (IIb):

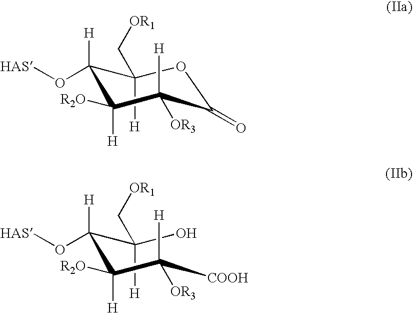

Although the oxidation may be carried out according to all suitable method or methods resulting in the oxidized reducing end of hydroxyalkyl starch, it is preferably carried out using an alkaline iodine solution as described, e.g., in DE 196 28 705 A1 the respective contents of which (example A, column 9, lines 6 to 24) is incorporated herein by reference.

The polymer derivative resulting from the reaction of the polymer with the at least bifunctional compound, is further reacted with the monohalogen-substituted acetic acid and/or a reactive monohalogen-substituted acetic acid derivative.

As monohalogen-substituted acetic acid or reactive acid, Cl-substituted, Br-substituted and I-substituted acetic acid are preferred, with acetic acid chloride being particularly preferred.

If the halogen-substituted acid is employed as such, it is preferred to react the acid with the polymer derivative in the presence of an activating agent. Suitable activating agents are, among others, Suitable activating agents are, among others, carbodiimides such as diisopropyl carbodimide (DIC), dicyclohexyl carbodiimides (DCC), 1-ethyl-3-(3-dimethylaminopropyl) carbodiimide (EDC), with dicyclohexyl carbodiimides (DCC) and 1-ethyl-3-(3-dimethylaminopropyl)carbodiimide (EDC) being especially preferred.

Therefore, the present invention also relates to a method and a conjugate as described above, wherein the polymer, preferably HES, is reacted with a diamino compound, preferably a diaminoalkane with 2 to 8 carbon atoms or H₂N—(CH₂—CH₂—O)ₘ—CH₂—CH₂—NH₂ with m=1, 2, 3, or 4, and reacting the resulting polymer derivative with Br-substituted and I-substituted acetic acid in the presence of an activating agent, preferably EDC.

Therefore, the present invention also relates to a polymer derivative according to the formula

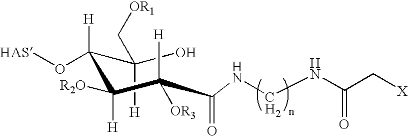

with X=Cl, Br or I, n=2, 3, 4, 5, 6, 7, or 8, and the polymer especially preferably being HES, or a polymer derivative according to the formula

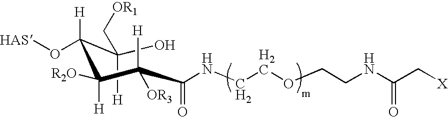

with X=Cl, Br or I, m=1, 2, 3, or 4, and the polymer especially preferably being HES.

The reaction of the polymer derivative with the halogen-substituted acetic acid is preferably carried out it in an aqueous system, preferably water, at a preferred pH of from 3.5 to 5.5, more preferably from 4.0 to 5.0 and especially preferably from 4.5 to 5.0; and a preferred reaction temperature of from 4 to 30° C., more preferably from 15 to 25 and especially preferably from 20 to 25° C.; and for a preferred reaction time of from 1 to 8 h, more preferably from 2 to 6 h and especially from 3 to 5 h.

The reaction mixture comprising the polymer derivative which comprises the polymer, the at least bifunctional compound and the halogen-substituted acetic acid, can be used for the reaction with the protein as such. According to a preferred embodiment of the present invention, the polymer derivative is separated from the reaction mixture, preferably by ultrafiltration, subsequent precipitation, optional washing and drying in vacuo.

The reaction of the polymer derivative with the protein is preferably carried out in an aqueous system.

The reaction of the polymer derivative with the protein is carried out at a preferred pH of from 6.5 to 8.5, more preferably from 7.0 to 8.5 and especially preferably from 7.5 to 8.5; and a preferred reaction temperature of from 4 to 30° C., more preferably from 15 to 25 and especially preferably from 20 to 25° C.; and for a preferred reaction time of from 0.5 to 8 h, more preferably from 1 to 6 h and especially from 2 to 5 h.

The reaction of the polymer derivative with the thiol group of the protein results in a thioether linkage between the polymer derivative and the protein.

Therefore, the present invention also relates to a method and a conjugate as described above, wherein the polymer, preferably HES, is reacted with a diamino compound, preferably a diaminoalkane with 2 to 8 carbon atoms or H₂N—(CH₂—CH₂—O)ₘ—CH₂—CH₂—NH₂ with m=1, 2, 3, or 4, the resulting polymer derivative is reacted with Br-substituted and I-substituted acetic acid in the presence of an activating agent, preferably EDC, and the resulting polymer derivative is reacted with a thiol group of the protein to give a conjugate comprising a thioether linkage between the protein and the polymer derivative.

Therefore, the present invention also relates to a conjugate according to the formula

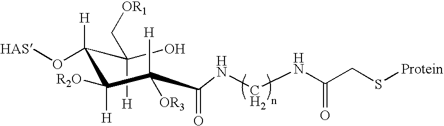

with n=2, 3, 4, 5, 6, 7, or 8, and the polymer especially preferably being HES, or a conjugate according to the formula

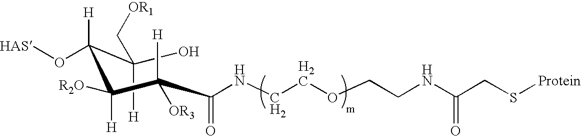

with m=1, 2, 3, or 4, and the polymer especially preferably being HES. The hydroxyethyl starch is preferably hydroxethyl starch having a mean molecular weight of about 10 kD and a DS of about 0.4 or hydroxethyl starch having a mean molecular weight of about 10 kD and a DS of about 0.7 or hydroxethyl starch having a mean molecular weight of about 12 kD and a DS of about 0.4 or hydroxethyl starch having a mean molecular weight of about 12 kD and a DS of about 0.7 or hydroxethyl starch having a mean molecular weight of about 18 kD and a DS of about 0.4 or hydroxethyl starch having a mean molecular weight of about 18 kD and a DS of about 0.7 or hydroxethyl starch having a mean molecular weight of about 50 kD and a DS of about 0.4 or hydroxethyl starch having a mean molecular weight of about 50 kD and a DS of about 0.7.

According to a second embodiment, functional group Z of the protein is a thiol group and functional group A of the polymer comprises a maleimido group.

According to this embodiment, several possibilities exist to produce the conjugate. In general, the polymer is reacted at its optionally oxidized reducing end with at least one at least bifunctional compound, wherein this at least bifunctional compound comprises one functional group which is capable of being reacted with the optionally oxidized reducing end of the polymer, and at least one functional group which either comprises the maleimido group or is chemically modified to give a polymer derivative which comprises the maleimido group. According to a preferred embodiment, said functional group is chemically modified to give a polymer derivative which comprises the maleimido group.

Therefore, the present invention relates to a method and a conjugate as described above, by reacting a polymer derivative comprising a maleimido group with a thiol group of the protein, said method comprising reacting the polymer at its optionally oxidized reducing end with an at least bifunctional compound comprising a functional group U capable of reacting with the optionally oxidised reducing end, the at least bifunctional compound further comprising a functional group W capable of being chemically modified to give a maleimido group, the method further comprising chemically modifying the functional group W to give a maleimido group.

As to functional group U, each functional group is conceivable which is capable of being reacted with optionally oxidised reducing end of the polymer.

According to a preferred embodiment of the present invention, the functional group U comprises the chemical structure —NH—.

Therefore, the present invention also relates to a method and a conjugate as described above, wherein the functional group U comprises the structure —NH—.

According to one preferred embodiment of the present invention, the functional group U is a group having the structure R′—NH— where R′ is hydrogen or a alkyl, cycloalkyl, aryl, aralkyl, arylcycloalkyl, alkaryl or cycloalkylaryl residue where the cycloalkyl, aryl, aralkyl, arylcycloalkyl, alkaryl or cycloalkylaryl residue may be linked directly to the NH group or, according to another embodiment, may be linked by an oxygen bridge to the NH group. The alkyl, cycloalkyl, aryl, aralkyl, arylcycloalkyl, alkaryl, or cycloalkylaryl residues may be suitably substituted. As preferred substituents, halogenes such as F, Cl or Br may be mentioned. Especially preferred residues R′ are hydrogen, alkyl and alkoxy groups, and even more preferred are hydrogen and unsubstituted alkyl and alkoxy groups.

Among the alkyl and alkoxy groups, groups with 1, 2, 3, 4, 5, or 6 C atoms are preferred. More preferred are methyl, ethyl, propyl, isopropyl, methoxy, ethoxy, propoxy, and isopropoxy groups. Especially preferred are methyl, ethyl, methoxy, ethoxy, and particular preference is given to methyl or methoxy.

Therefore, the present invention also relates to a method and a conjugate as described above, wherein R′ is hydrogen or a methyl or a methoxy group.

According to another preferred embodiment of the present invention, the functional group U has the structure R′—NH—R″— where R″ preferably comprises the structure unit —NH— and/or the structure unit —(C=G)- where G is O or S, and/or the structure unit —SO₂—. According to more preferred embodiments, the functional group R″ is selected from the group consisting of

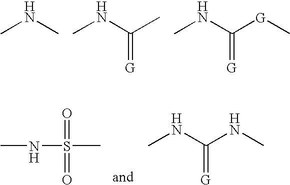

where, if G is present twice, it is independently O or S.

Therefore, the present invention also relates to a method and a conjugate as described above, wherein the functional group U is selected from the group consisting of

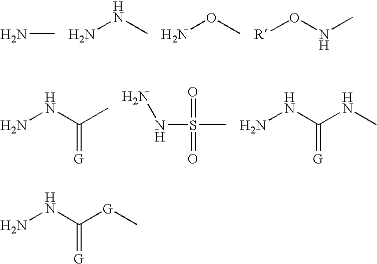

wherein G is O or S and, if present twice, independently O or S, and R′ is methyl.

According to a still more preferred embodiment of the present invention, U comprises an amino group —NH₂.

According to an embodiment of the present invention, the functional group W of the at least bifunctional compound is chemically modified by reacting the polymer derivative comprising W with a further at least bifunctional compound comprising a functional group capable of being reacted with W and further comprising a maleimido group.

As to functional group W and the functional group of said further at least bifunctional compound which is capable of being reacted with W, the following functional groups are to be mentioned, among others:
C—C-double bonds or C—C-triple bonds or aromatic C—C-bonds;
the thio group or the hydroxy groups;
alkyl sulfonic acid hydrazide, aryl sulfonic acid hydrazide;
1,2-dioles;
1,2-aminoalcohols;
1,2 amino-thioalcohols;
azides;
the amino group —NH₂ or derivatives of the amino groups comprising the structure unit —NH— such as aminoalkyl groups, aminoaryl group, aminoaralkyl groups, or alkarlyamino groups;
the hydroxylamino group —O—NH₂, or derivatives of the hydroxylamino group comprising the structure unit —O—NH—, such as hydroxylalkylamino groups, hydroxylarylamino groups, hydroxylaralkylamino groups, or hydroxalalkarylamino groups;
alkoxyamino groups, aryloxyamino groups, aralkyloxyamino groups, or alkaryloxyamino groups, each comprising the structure unit —NH—O—;
residues having a carbonyl group, -Q-C(=G)-M, wherein G is O or S, and M is, for example,
   —OH or —SH;
   an alkoxy group, an aryloxy group, an aralkyloxy group, or an alkaryloxy group;
   an alkylthio group, an arylthio group, an aralkylthio group, or an alkarylthio group;
   an alkylcarbonyloxy group, an arylcarbonyloxy group, an aralkylcarbonyloxy group, an alkarylcarbonyloxy group;
   activated esters such as esters of hydroxyl amines having imid structure such as N-hydroxysuccinimide or having a structure unit O—N where N is part of a heteroaryl compound or, with G=O and Q absent, such as aryloxy compounds with a substituted aryl residue such as pentafluorophenyl, paranitrophenyl or trichlorophenyl;
wherein Q is absent or NH or a heteroatom such as S or O;
   —NH—NH₂, or —NH—NH—;
   —NO₂;
   the nitril group;
   carbonyl groups such as the aldehyde group or the keto group;
   the carboxy group;
   the —N═C═O group or the —N═C═S group;
   vinyl halide groups such as the vinyl iodide or the vinyl bromide group or triflate;
   —C≡C—H;
   —(C═NH₂Cl)—OAlkyl
   groups —(C═O)—CH₂-Hal wherein Hal is Cl, Br, or I;
   —CH═CH—SO₂—;
   a disulfide group comprising the structure —S—S—;
   the group

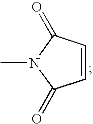

the group

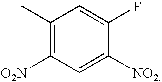

where W and the functional group of the further at least bifunctional compound, respectively, is a group capable of forming a chemical linkage with one of the above-mentioned groups.

According to a still more preferred embodiment of the present invention, W comprises an amino group —NH₂.

According to preferred embodiments of the present invention, both W and the other functional group are groups from the list of groups given above.

According to one embodiment of the present invention, one of these functional groups is a thio group. In this particular case, the other functional group is preferably selected from the group consisting of

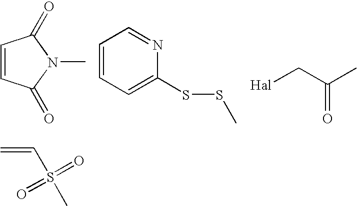

wherein Hal is Cl, Br, or I, preferably Br or I.

According to an especially preferred embodiment of the present invention, one of these functional groups is selected from the group consisting of a reactive ester such as an ester of hydroxyl amines having imide structure such as N-hydroxysuccinimide or having a structure unit O—N where N is part of a heteroaryl compound or such as an aryloxy compound with a substituted aryl residue such as pentafluorophenyl, paranitrophenyl or trichlorophenyl, or a carboxy group which is optionally transformed into a reactive ester. In this particular case, the other functional group comprises the chemical structure —NH—.

According to an especially preferred embodiment of the present invention, W comprises the structure —NH— and the further at least bifunctional compound comprises a reactive ester and the maleimido group.

As to the functional group W comprising the structure —NH—, reference can be made to the functional group as described above, wherein W may be the same or different from U. According to a preferred embodiment of the present invention, U and W are the same. More preferably, both U and W comprise an amino group. Particularly preferred, both U and W are an amino group —NH₂.

According to one embodiment of the present invention, the polymer may be reacted with the at least bifunctional compound comprising U and W at its non-oxidized reducing end in an aqueous medium. According to a preferred embodiment where U and W both are an amino group, the reaction is carried out using the polymer with the reducing end in the oxidized form, in at least one aprotic solvent, particularly preferably in an anhydrous aprotic solvent having a water content of not more than 0.5 percent by weight, preferably of not more than 0.1 percent by weight. Suitable solvents are, among others, dimethyl sulfoxide (DMSO), N-methylpyrrolidone, dimethyl acetamide (DMA), dimethyl formamide (DMF) and mixtures of two or more thereof.

Especially in case both U and W are an amino group —NH₂, U and W may be separated by any suitable spacer. Among others, the spacer may be an optionally substituted, linear, branched and/or cyclic hydrocarbon residue. Suitable substituents are, among others, alkyl, aryl, aralkyl, alkaryl, halogen, carbonyl, acyl, carboxy, carboxyester, hydroxy, thio, alkoxy and/or alkylthio groups. Generally, the hydrocarbon residue has from 1 to 60, preferably from 1 to 40, more preferably from 1 to 20, more preferably from 2 to 10, more preferably from 2 to 6 and especially preferably from 2 to 4 carbon atoms. If heteroatoms are present, the separating group comprises generally from 1 to 20, preferably from 1 to 8 and especially preferably from 1 to 4 heteroatoms. The hydrocarbon residue may comprise an optionally branched alkyl chain or an aryl group or a cycloalkyl group having, e.g., from 5 to 7 carbon atoms, or be an aralkyl group, an alkaryl group where the alkyl part may be a linear and/or cyclic alkyl group. According to an even more preferred embodiment, the hydrocarbon residue is an alkyl chain of from 1 to 20, preferably from 2 to 10, more preferably from 2 to 6, and especially preferably from 2 to 4 carbon atoms.

Therefore, the present invention also relates to a method and a conjugate as described above, wherein the polymer is reacted with its oxidized reducing end with 1,4-diaminobutane, 1,3-diaminopropane or 1,2-diaminoethane to give a polymer derivative according to the formula

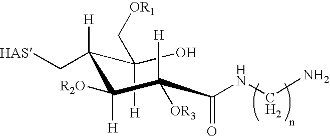

with n=2, 3, or 4, the polymer preferably being HES.

According to the above-mentioned preferred embodiment, the polymer derivative comprising an amino group is further reacted with an at least bifunctional compound comprising a reactive ester group and the maleimido group. The reactive ester group and the maleimido group may be separated by a suitable spacer. As to this spacer, reference can be made to the spacer between the functional groups U and W. According to a preferred embodiment of the present invention, the reactive ester group and the maleimido group are separated by a hydrocarbon chain having from 1 to 10, preferably from 1 to 8, more preferably from 1 to 6, more preferably from 1 to 4, more preferably from 1 to 2 and particularly preferably 1 carbon atom. According to a still further preferred embodiment, the reactive ester is a succinimide ester, and according to a particularly preferred embodiment, the at least bifunctional compound comprising the maleimido group and the reactive ester group is N-(alpha-maleimidoacetoxy)succinimide ester.

Therefore, the present invent also relates to a polymer derivative according to the formula

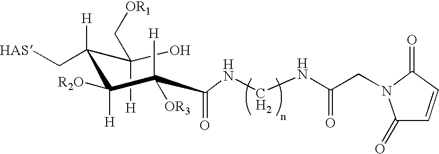

with n 2, 3, or 4, the polymer preferably being HES.

The polymer derivative comprising the maleimido group is further reacted with the thiol group of the protein to give a conjugate comprising the polymer derivative linked to the protein via a thioether group.

Therefore, the present invention also relates to a conjugate, comprising the protein and the polymer, according to the formula

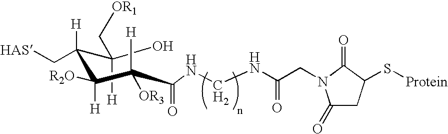

with n=2, 3, or 4, preferably 4, the polymer preferably being HES, and wherein the S atom in the formula above derives from Cys17 or Cys18 of the protein. The hydroxyethyl starch is preferably hydroxethyl starch having a mean molecular weight of about 10 kD and a DS of about 0.4 or hydroxethyl starch having a mean molecular weight of about 10 kD and a DS of about 0.7 or hydroxethyl starch having a mean molecular weight of about 12 kD and a DS of about 0.4 or hydroxethyl starch having a mean molecular weight of about 12 kD and a DS of about 0.7 or hydroxethyl starch having a mean molecular weight of about 18 kD and a DS of about 0.4 or hydroxethyl starch having a mean molecular weight of about 18 kD and a DS of about 0.7 or hydroxethyl starch having a mean molecular weight of about 50 kD and a DS of about 0.4 or hydroxethyl starch having a mean molecular weight of about 50 kD and a DS of about 0.7.

The reaction of the polymer derivative comprising the maleimido group with the thiol group of the protein is preferably carried in a buffered aqueous system, at a preferred pH of from 5.5 to 8.5, more preferably from 6 to 8 and especially preferably from 6.5 to 7.5, and a preferred reaction temperature of from 6 to 40° C., more preferably from 0 to 25 and especially preferably from 4 to 21° C., and for a preferred reaction time of from 0.5 to 24 h, more preferably from 1 to 20 h and especially from 2 to 17 h. The suitable pH value of the reaction mixture may be adjusted by adding at least one suitable buffer. Among the preferred buffers, sodium acetate buffer, phosphate or borate buffers may be mentioned, containing either urea at a preferred concentration of from 0 to 8 M, more preferred from 2 to 8 M and especially preferred from 4 to 8 M, and/or containing SDS at a preferred concentration of from 0 to 1% (w/v), more preferred from 0.4 to 1% (w/v) and especially proffered from 0.8 to 1% (w/v).

The conjugate may be subjected to a further treatment such as an after-treatment like dialysis, centrifugal filtration or pressure filtration, ion exchange chromatography, reversed phase chromatography, HPLC, MPLC, gel filtration and/or lyophilisation.

Therefore, the present invention also relates to a conjugate as obtainable by a method as described above.

Therefore, the present invention also relates to a conjugate as obtainable by a method as described above, wherein A is a reactive carboxy group, and wherein A was introduced in the polymer whose reducing end was not oxidized, by reacting at least one hydroxy group of the polymer with a carbonic diester, and wherein, said comprising one polymer molecule and at least one, in particular of from 1 to 10 protein molecules linked to the polymer via amide linkages.

The present invention also relates to a conjugate comprising a protein and a polymer or a derivative thereof, wherein the polymer is a hydroxyalkyl starch (HAS) and the protein is a granulocyte colony stimulating factor (G-CSF), having a structure according to the formula

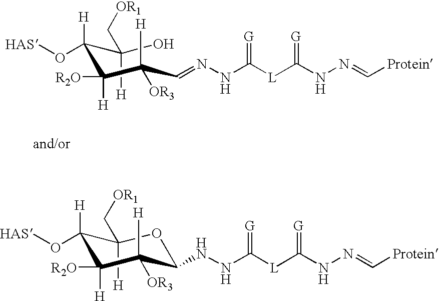

wherein R₁, R₂ and R₃ are independently hydrogen or a hydroxyalkyl group, a hydroxyaryl group, a hydroxyaralkyl group or a hydroxyalkaryl group having of from 1 to 10 carbon atoms, preferably hydrogen or a hydroxyalkyl group, more preferably hydrogen or a hydroxyethyl group,
wherein G is selected from the group consisting of O and S, preferably O, and wherein L is an optionally suitably substituted, linear, branched and/or cyclic hydrocarbon residue, optionally comprising at least one heteroatom, preferably an alkyl, aryl, aralkyl, heteroaryl, heteroaralkyl residue having from 2 to 60 carbon atoms.

The abbreviation “Protein′” as used in the formulae above refers to the G-CSF molecule used for the reaction without the carbon atom of the carbohydrate moiety which is part of oxime linkage in the N═C double bond.

The present invention also relates to a conjugate as described above, wherein -L- is —(CH₂)n- with n=2, 3, 4, 5, 6, 7, 8, 9, 10, preferably 2, 3, 4, 5, 6, more preferably 2, 3, 4, and especially preferably 4.

The present invention also relates to a conjugate comprising a protein and a polymer or a derivative thereof, wherein the polymer is a hydroxyalkyl starch (HAS) and the protein is a granulocyte colony stimulating factor (G-CSF), having a structure according to the formula

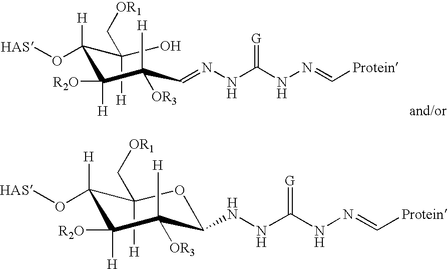

wherein R₁, R₂ and R₃ are independently hydrogen or a hydroxyalkyl group, a hydroxyaryl group, a hydroxyaralkyl group or a hydroxyalkaryl group having of from 1 to 10 carbon atoms, preferably hydrogen or a hydroxyalkyl group, more preferably hydrogen or a hydroxyethyl group, and
wherein G is selected from the group consisting of O and S, preferably O, and

The abbreviation “Protein′” as used in the formulae above refers to the G-CSF molecule used for the reaction without the carbon atom of the carbohydrate moiety which is part of oxime linkage in the N═C double bond.

The present invention also relates to a conjugate comprising a protein and a polymer or a derivative thereof, wherein the polymer is a hydroxyalkyl starch (HAS) and the protein is a granulocyte colony stimulating factor (G-CSF), having a structure according to the formula

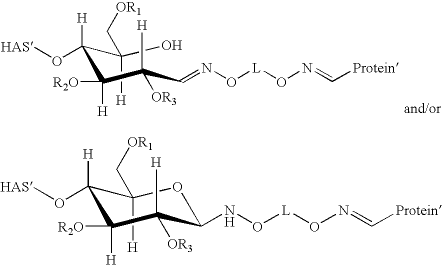

wherein R₁, R₂ and R₃ are independently hydrogen or a hydroxyalkyl group, a hydroxyaryl group, a hydroxyaralkyl group or a hydroxyalkaryl group having of from 1 to 10 carbon atoms, preferably hydrogen or a hydroxyalkyl group, more preferably hydrogen or a hydroxyethyl group, and
wherein L is an optionally suitably substituted, linear, branched and/or cyclic hydrocarbon residue, optionally comprising at least one heteroatom, preferably an alkyl, aryl, aralkyl, heteroaryl, heteroaralkyl residue having from 2 to 60 carbon atoms.

The abbreviation “Protein′” as used in the formulae above refers to the G-CSF molecule used for the reaction without the carbon atom of the carbohydrate moiety which is part of oxime linkage in the N═C double bond.

The two structures above describe a structure where the crosslinking compound is linked via an oxime linkage to the reducing end of HAS where the terminal saccharide unit of HES is present in the open form, and a structure with the respective cyclic animal form where the crosslinking compound is linked to the reducing end of HES via an oxyamino group and where the terminal saccharide unit of HES is present in the cyclic form. Both structures may be simultaneously present in equilibrium with each other.

The present invention also relates to a conjugate as described above, wherein -L- is

—[(CRₐR_{b})ₘG]ₙ[CR_{c}R_{d}]ₒ—

wherein Rₐ; R_{b}, R_{c}, R_{d} are independently hydrogen, alkyl, aryl, preferably hydrogen, wherein G is selected from the group consisting of O and S, preferably O,
and wherein
m 1, 2, 3 or 4, wherein the residues Rₐ and R_{b} may be the same or different in the m groups CRₐR_{b};
n 0 to 20, preferably 0 to 10, more preferably 1, 2, 3, 4, 5, most preferably 1 or 2;
o 0 to 20, preferably 0 to 10, more preferably 1, 2, 3, 4, 5, most preferably 1 or 2, wherein the residues R_{c} and R_{d} may be the same or different in the o groups CR_{c}R_{d};

The present invention also relates to a conjugate as described above, wherein Rₐ; R_{b}, R_{c}, R_{d} are hydrogen, m=2, n=1, and o=2.

The present invention also relates to a conjugate comprising a protein and a polymer or a derivative thereof, wherein the polymer is a hydroxyalkyl starch (HAS) and the protein is a granulocyte colony stimulating factor (G-CSF), having a structure according to the formula

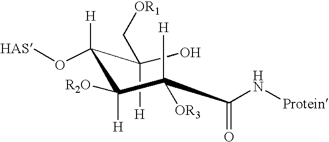

wherein R₁, R₂ and R₃ are independently hydrogen or a hydroxyalkyl group, a hydroxyaryl group, a hydroxyaralkyl group or a hydroxyalkaryl group having of from 1 to 10 carbon atoms, preferably hydrogen or a hydroxyalkyl group, more preferably hydrogen or a hydroxyethyl group.

The abbreviation “Protein′” as used in the formula above refers to the G-CSF molecule used for the reaction without the nitrogen atom of the amino group which is part the amide linkage.

The present invention also relates to a conjugate comprising a protein and a polymer or a derivative thereof, wherein the polymer is a hydroxyalkyl starch (HAS) and the protein is a granulocyte colony stimulating factor (G-CSF), having a structure according to the formula

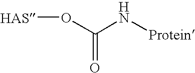

wherein R₁, R₂ and R₃ are independently hydrogen or a hydroxyalkyl group, a hydroxyaryl group, a hydroxyaralkyl group or a hydroxyalkaryl group having of from 1 to 10 carbon atoms, preferably hydrogen or a hydroxyalkyl group, more preferably hydrogen or a hydroxyethyl group, and
wherein the linkage —O—(C═O)— was formed by a reaction of a carboxy group or a reactive carboxy group with a hydroxy group of the HAS molecule.

The abbreviation “Protein′” as used in the formula above refers to the G-CSF molecule used for the reaction without the nitrogen atom of the amino group which is part the amide linkage.

The present invention also relates to a conjugate, comprising a protein and a polymer or a derivative thereof, wherein the polymer is a hydroxyalkyl starch (HAS) and the protein is a granulocyte colony stimulating factor (G-CSF), having a structure according to the formula

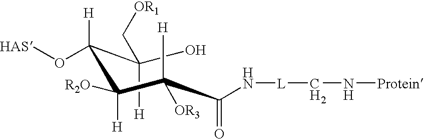

wherein R₁, R₂ and R₃ are independently hydrogen or a hydroxyalkyl group, a hydroxyaryl group, a hydroxyaralkyl group or a hydroxyalkaryl group having of from 1 to 10 carbon atoms, preferably hydrogen or a hydroxyalkyl group, more preferably hydrogen or a hydroxyethyl group, and
wherein L is an optionally substituted, linear, branched and/or cyclic hydrocarbon residue, optionally comprising at least one heteroatom, having from 1 to 60 preferably from 1 to 40, more preferably from 1 to 20, more preferably from 1 to 10, more preferably from 1 to 6 more preferably from 1 to 2 carbon atoms and especially preferably 1 carbon atom, L being in particular CH₂.

The abbreviation “Protein′” as used in the formula above refers to the G-CSF molecule used for the reaction without the nitrogen atom of the amino group which is part the aminomethyl linkage.

The present invention also relates to a conjugate, comprising a protein and a polymer or a derivative thereof, wherein the polymer is a hydroxyalkyl starch (HAS) and the protein is a granulocyte colony stimulating factor (G-CSF), having a structure according to the formula

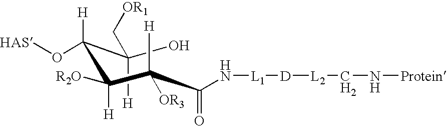

wherein R₁, R₂ and R₃ are independently hydrogen or a hydroxyalkyl group, a hydroxyaryl group, a hydroxyaralkyl group or a hydroxyalkaryl group having of from 1 to 10 carbon atoms, preferably hydrogen or a hydroxyalkyl group, more preferably hydrogen or a hydroxyethyl group, and
wherein L₁ and L₂ are independently an optionally substituted, linear, branched and/or cyclic hydrocarbon residue, optionally comprising at least one heteroatom, comprising an alkyl, aryl, aralkyl heteroalkyl, and/or heteroaralkyl moiety, said residue having from 1 to 60 preferably from 1 to 40, more preferably from 1 to 20, more preferably from 1 to 10 carbon atoms, and
wherein D is a linkage, preferably a covalent linkage which was formed by a suitable functional group F² linked to L1 and a suitable functional group F₃ linked to L₂.

The abbreviation “Protein′” as used in the formulae above refers to the G-CSF molecule used for the reaction without the nitrogen atom of the amino group which is part of the aminomethyl linkage.

The present invention also relates to a conjugate as described above, wherein L₁ is —(CH₂)n- with n=2, 3, 4, 5, 6, 7, 8, 9, 10, preferably 2, 3, 4, 5, 6, more preferably 2, 3, 4, and especially preferably 4.

The present invention also relates to a conjugate as described above, wherein L₂ comprises an optionally suitably substituted aryl moiety, preferably an aryl moiety containing 6 carbon atoms, L₂ being especially preferably C₆H₄, or wherein L₂ is —(CH₂)ₙ— with n=2, 3, 4, 5, 6, 7, 8, 9, 10, preferably 2, 3, 4, 5, 6, more preferably 2, 3, 4.

The present invention also relates to a conjugate as described above, wherein is selected from the group consisting of
—C—C-double bonds or C—C-triple bonds or aromatic C—C-bonds;
the thio group or the hydroxy groups;
alkyl sulfonic acid hydrazide, aryl sulfonic acid hydrazide;
1,2-dioles;
1,2 amino-thioalcohols;
azides;
1,2-aminoalcohols;
the amino group —NH₂ or derivatives of the amino groups comprising the structure unit —NH— such as aminoalkyl groups, aminoaryl group, aminoaralkyl groups, or alkarlyamino groups;
the hydroxylamino group —O—NH₂, or derivatives of the hydroxylamino group comprising the structure unit —O—NH—, such as hydroxylalkylamino groups, hydroxylarylamino groups, hydroxylaralkylamino groups, or hydroxalalkarylamino groups;
alkoxyamino groups, aryloxyamino groups, aralkyloxyamino groups, or alkaryloxyamino groups, each comprising the structure unit —NH—O—;
residues having a carbonyl group, -Q-C(=G)-M, wherein G is O or S, and M is, for example,
   —OH or —SH;
   an alkoxy group, an aryloxy group, an aralkyloxy group, or an alkaryloxy group;
   an alkylthio group, an arylthio group, an aralkylthio group, or an alkarylthio group;
   an alkylcarbonyloxy group, an arylcarbonyloxy group, an aralkylcarbonyloxy group, an alkarylcarbonyloxy group;
   activated esters such as esters of hydroxyl amines having imid structure such as N-hydroxysuccinimide or having a structure unit O—N where N is part of a heteroaryl compound or, with G=O and Q absent, such as aryloxy compounds with a substituted aryl residue such as pentafluorophenyl, paranitrophenyl or trichlorophenyl;
      wherein Q is absent or NH or a heteroatom such as S or O;
   —NH—NH₂, or —NH—NH—;
   —NO₂;
   the nitril group;
   carbonyl groups such as the aldehyde group or the keto group;
   the carboxy group;
   the —N═C═O group or the —N═C═S group;
   vinyl halide groups such as the vinyl iodide or the vinyl bromide group or triflate;
   —C≡C—H;
   —(C═NH₂Cl)—OAlkyl
   groups —(C═O)—CH₂-Hal wherein Hal is Cl, Br, or I;
   —CH═CH—SO₂—;
   a disulfide group comprising the structure —S—S—;
   the group

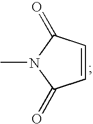

the group

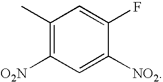

and wherein F₃ is a functional group capable of forming a chemical linkage with F₂ and is preferably selected from the above-mentioned group, F₂ preferably comprising the moiety —NH—, more preferably comprising an amino group, F₃ preferably comprising the moiety —(C=G)-, more preferably —(C═O)—, more preferably the moiety —(C=G)-G-, still more preferably —(C═O)-G-, and especially preferably —(C═O)—O, D being particularly preferably an amide linkage.

The present invention also relates to a conjugate comprising a protein and a polymer or a derivative thereof, wherein the polymer is a hydroxyalkyl starch (HAS) and the protein is a granulocyte colony stimulating factor (G-CSF), having a structure according to the formula

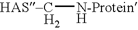

wherein the carbon atom of the moiety —CH₂—N2- is derived from an aldehyde group which was introduced in the polymer by a ring-opening oxidation reaction, wherein HAS″ relates to the hydroxyalkyl starch molecule without the aldehyde group resulting from the ring-opening oxidation and reacted with the amino group of the protein, and wherein the nitrogen atom is derived from an amino group of the protein.

The abbreviation “Protein′” as used in the formula above refers to the G-CSF molecule used for the reaction without the nitrogen atom of the amino group which is part the amide linkage.

The present invention also relates to a conjugate, comprising a protein and a polymer or a derivative thereof, wherein the polymer is a hydroxyalkyl starch (HAS) and the protein is a granulocyte colony stimulating factor (G-CSF), having a structure according to the formula

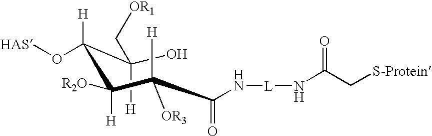

wherein R₁, R₂ and R₃ are independently hydrogen or a hydroxyalkyl group, a hydroxyaryl group, a hydroxyaralkyl group or a hydroxyalkaryl group having of from 1 to 10 carbon atoms, preferably hydrogen or a hydroxyalkyl group, more preferably hydrogen or a hydroxyethyl group, and
wherein L is an optionally substituted, linear, branched and/or cyclic hydrocarbon residue, optionally comprising at least one heteroatom, comprising an alkyl, aryl, aralkyl heteroalkyl, and/or heteroaralkyl moiety, said residue having from 2 to 60 preferably from 2 to 40, more preferably from 2 to 20, more preferably from 2 to 10 carbon atoms, and wherein the sulfur atom is derived from a cysteine residue or a disulfide group of the protein.

The present invention also relates to a conjugate as described above, wherein -L- is

—[(CRₐR_{b})ₘG]ₙ[CR_{c}R_{d}]ₒ—
wherein Rₐ; R_{b}, R_{c}; R_{d} are independently hydrogen, alkyl, aryl, preferably hydrogen,
wherein G is selected from the group consisting of O and S, preferably O, and
wherein
m 1, 2, 3 or 4, most preferably 21 wherein the residues Rₐ and R_{b} may be the same or different in the m groups CRₐR_{b};
n 1 to 20, preferably 1 to 10, most preferably 1, 2, 3, or 4;
o 1 to 20, preferably 1 to 10, more preferably 1, 2, 3, 4, 5, more preferably 1 or 2, most preferably 1, wherein the residues R_{c} and R_{d} may be the same or different in the o groups CR_{c}R_{d};
or
wherein
n 0, and
o 2 to 20, preferably 2 to 10, more preferably 2, 3, 4, 5, 6, 7, or 8, wherein the residues R_{c} and R_{d} may be the same or different in the o groups CR_{c}R_{d}.

The present invention also relates to a conjugate, comprising a protein and a polymer or a derivative thereof, wherein the polymer is a hydroxyalkyl starch (HAS) and the protein is a granulocyte colony stimulating factor (G-CSF), having a structure according to the formula

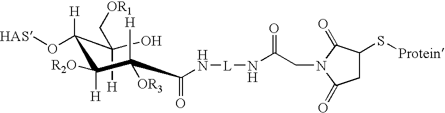

wherein R₁, R₂ and R₃ are independently hydrogen or a hydroxyalkyl group, a hydroxyaryl group, a hydroxyaralkyl group or a hydroxyalkaryl group having of from 1 to 10 carbon atoms, preferably hydrogen or a hydroxyalkyl group, more preferably hydrogen or a hydroxyethyl group, and
wherein L is an optionally substituted, linear, branched and/or cyclic hydrocarbon residue, optionally comprising at least one heteroatom, comprising an alkyl, aryl, aralkyl heteroalkyl, and/or heteroaralkyl moiety, said residue having from 2 to 60 preferably from 2 to 40, more preferably from 2 to 20, more preferably from 2 to 10 carbon atoms, and
wherein the sulfur atom is derived from a cysteine residue or a disulfide group of the protein.

The present invention also relates to a conjugate as described above, wherein -L- is

—[(CRₐR_{b})ₘG]ₙ[CR_{c}R_{d}]ₒ—
wherein Rₐ; R_{b}, R_{d}. R_{b} are independently hydrogen, alkyl, aryl, preferably hydrogen,
wherein G is selected from the group consisting of O and S, preferably O, and
wherein
m 1, 2, 3 or 4, most preferably 2, wherein the residues Rₐ and R_{b} may be the same or different in the m groups CRₐR_{b};
n 1 to 20, preferably 1 to 10, most preferably 1, 2, 3, or 4;
o 1 to 20, preferably 1 to 10, more preferably 1, 2, 3, 4, 5, more preferably 1 or 2, most preferably 1, wherein the residues R_{b} and R_{d} may be the same or different in the o groups CR_{b}R_{d};
or
wherein
n 0, and
o 2 to 20, preferably 2 to 10, more preferably 2, 3, 4, 5, 6, 7, or 8, wherein the residues R_{b} and R_{d} may be the same or different in the o groups CR_{b}R_{d}.

The present invention also relates to any conjugate as described above, wherein the hydroxyalkyl starch is hydroxyethyl starch.

The present invention also relates to any conjugate as described above, wherein the hydroxyethyl starch has a molecular weight of from 2 to 200 kD, preferably of from 4 to 130 kD, more preferably of from 4 to 70 kD.

According to a further aspect, the present invention relates to a conjugate as described above, or a conjugate, obtainable by a method as described above, for use in a method for the treatment of the human or animal body.

Furthermore, the present invention relates to a pharmaceutical composition comprising in a therapeutically effective amount a conjugate as described above or a conjugate, obtainable by a method as described above.

The term “in a therapeutically effective amount” as used in the context of the present invention refers to that amount which provides therapeutic effect for a given condition and administration regimen. The administration is preferably by routes. The specific route chosen will depend upon the condition being treated. The administration is preferably done as part of a formulation containing a suitable carrier, such as polysorbat, a suitable diluent, such as water and/or a suitable adjuvant such as sorbitol. The required dosage will depend upon the severity of the condition being treated, the patients individual response, the method of administration used, and the like.

Thus, in a preferred embodiment, the pharmaceutical composition further comprises at least one pharmaceutically acceptable diluent, adjuvant and/or carrier, especially preferably useful in G-CSF therapy.

The pharmaceutical composition is preferably used for the treatment of a disorder characterized by a reduced hematopoietic or immune function or diseases related thereto.

Therefore, the present invention also relates to the use of a pharmaceutical composition as described above, comprising a conjugate as described above or a conjugate, obtainable by a method as described above, for the preparation of a medicament for the treatment of a disorder characterized by a reduced hematopoietic or immune function.

According to a preferred embodiment, the disorder characterized by a reduced hematopoietic or immune function, is a result of chemotherapy, radiation therapy, infectious disease, severe chronic neutropenia, or leukemia. Therefore, the present invention also relates to the use of a pharmaceutical composition as described above, comprising a conjugate as described above or a conjugate, obtainable by a method as described above, for the preparation of a medicament for the treatment of a disorder characterized by a reduced hematopoietic or immune function, wherein the disorder is a result of chemotherapy, radiation therapy, infectious disease, severe chronic neutropenia, or leukemia.

The object of the treatment with the pharmaceutical composition according to the invention is preferably administered by i.v. or s.c. routes. For this, the pharmaceutical composition may be administered as a sterile solution.

The invention is further illustrated by the following figures, tables and examples, which are in no way intended to restrict the scope of the present invention.

### SHORT DESCRIPTION OF THE FIGURES

FIG. 1a

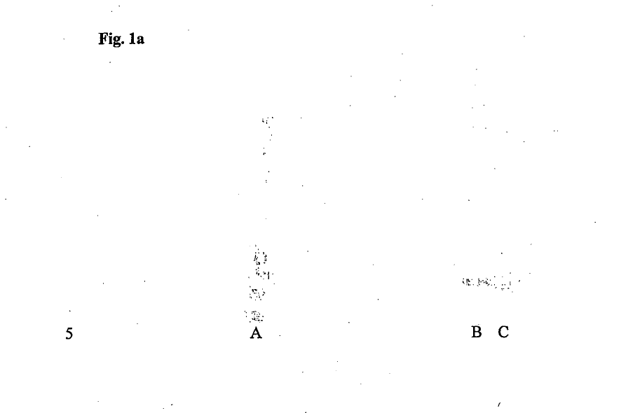

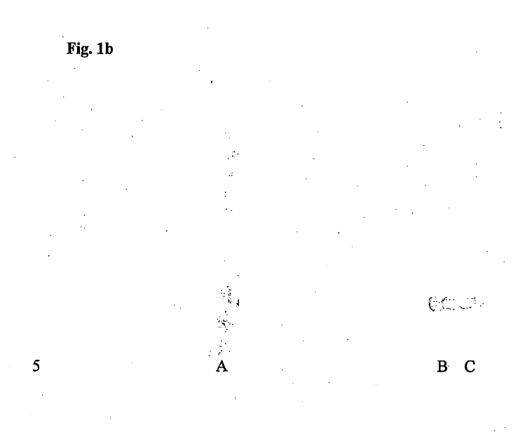

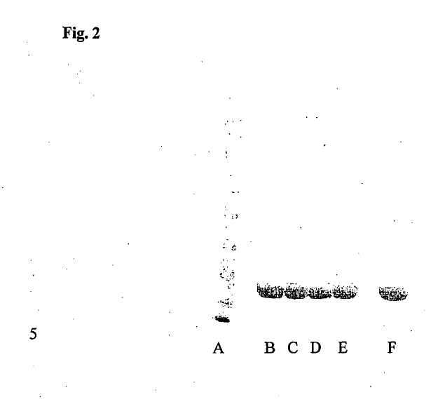

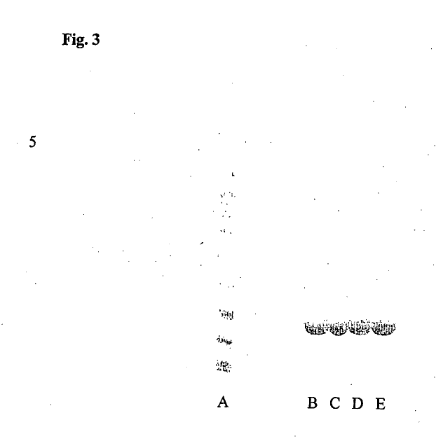

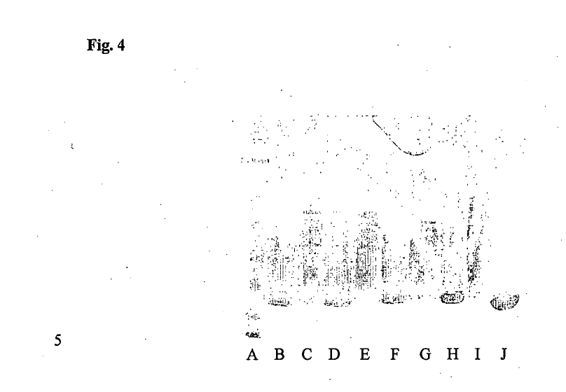

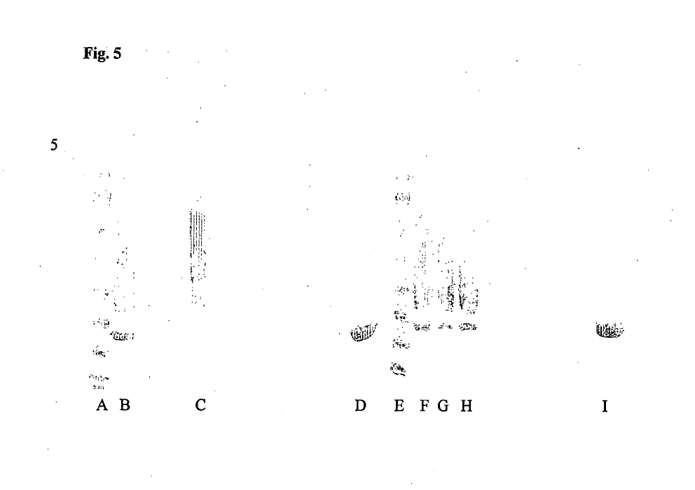

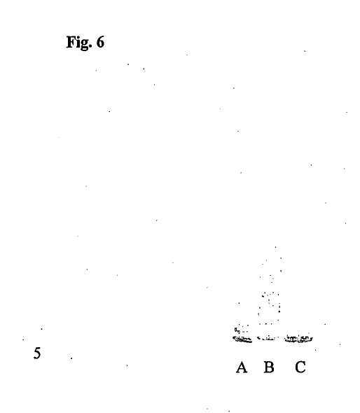

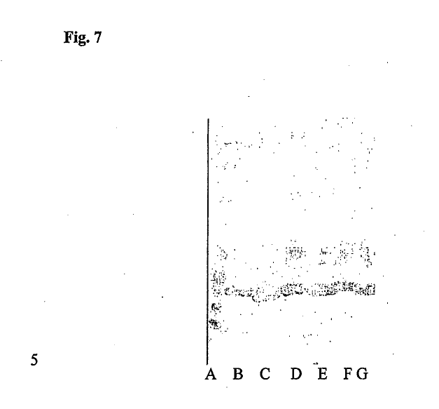

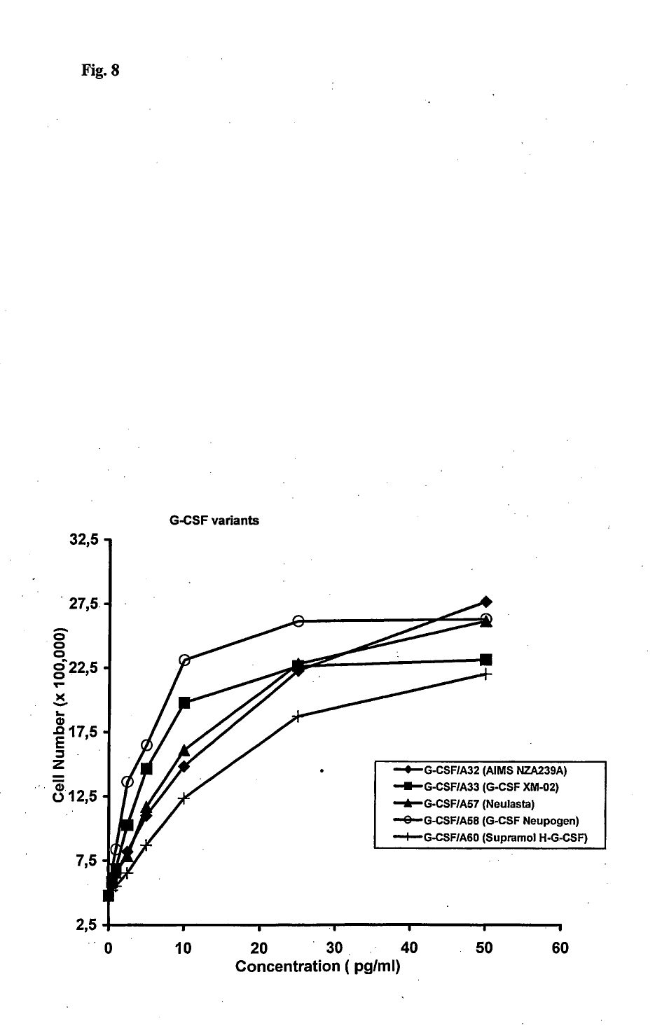

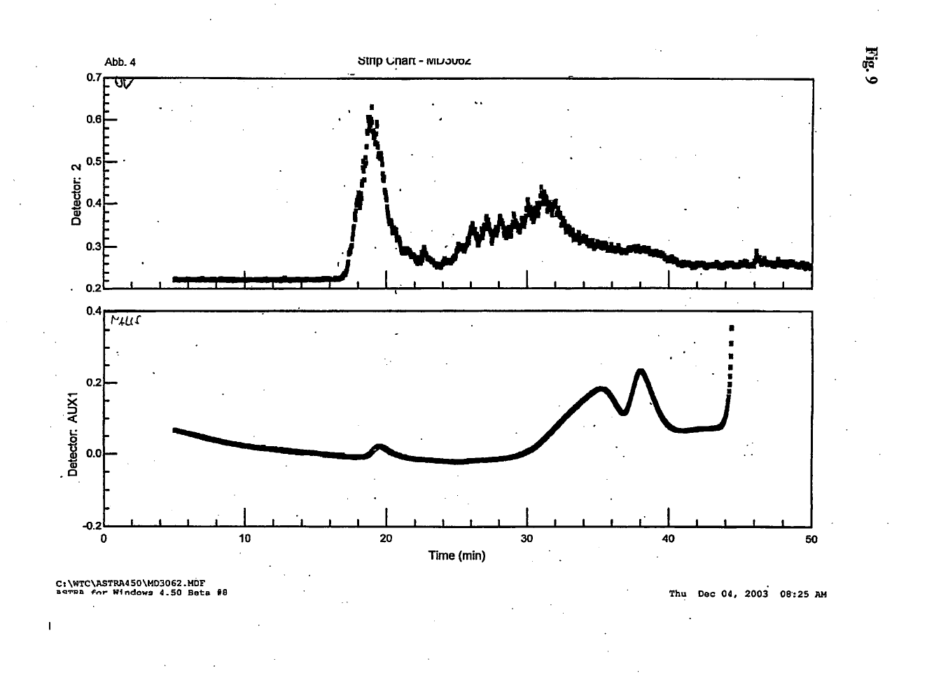

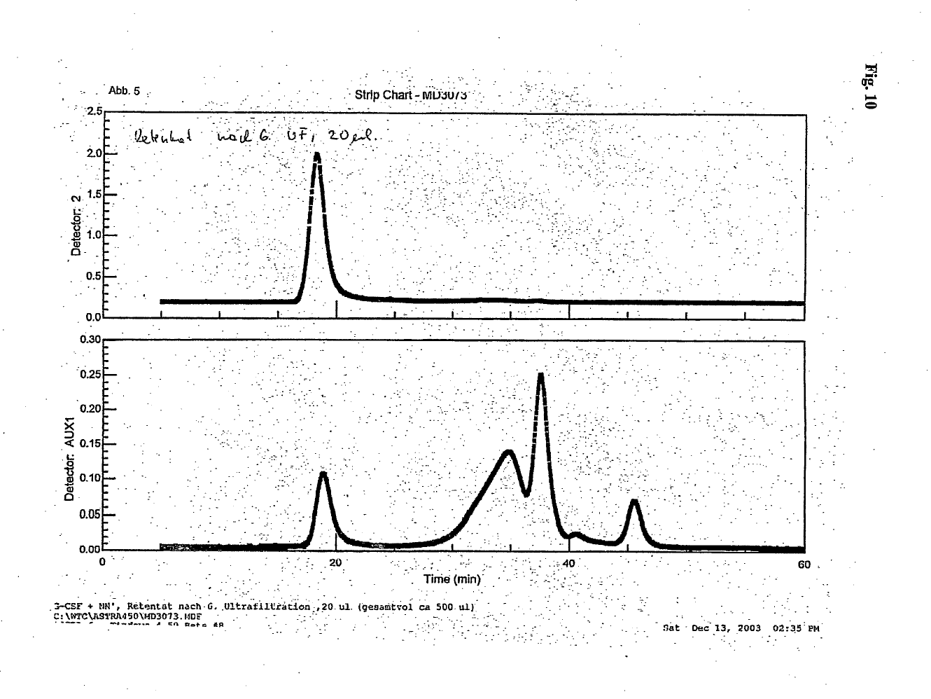

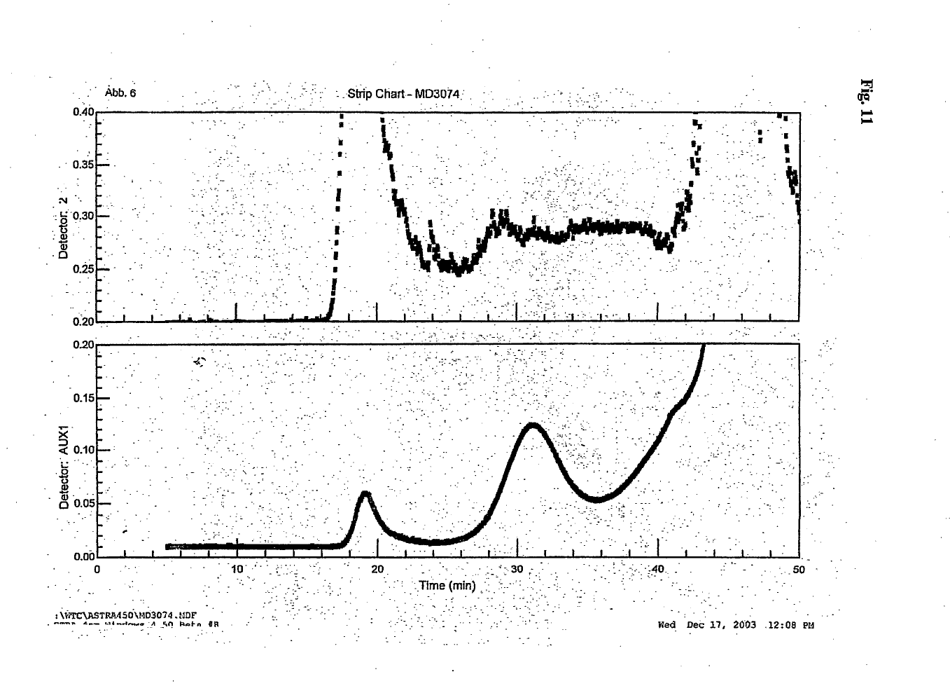

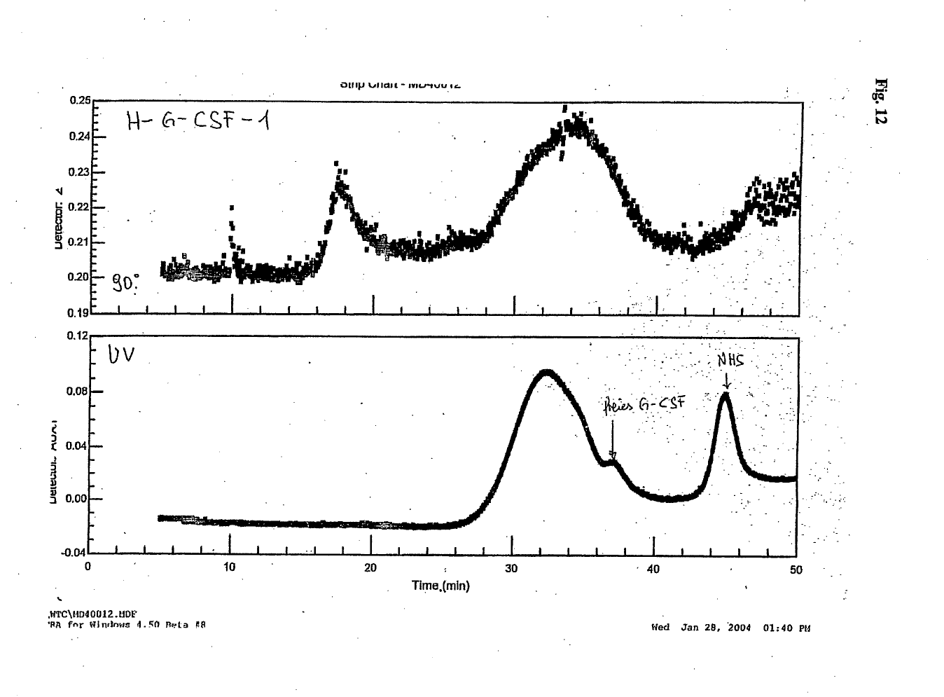

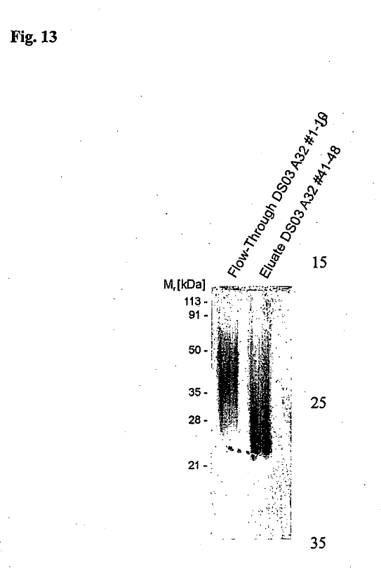

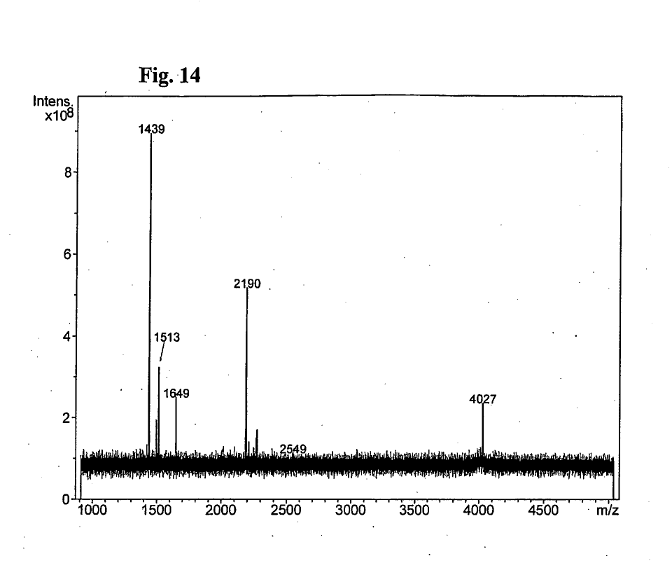

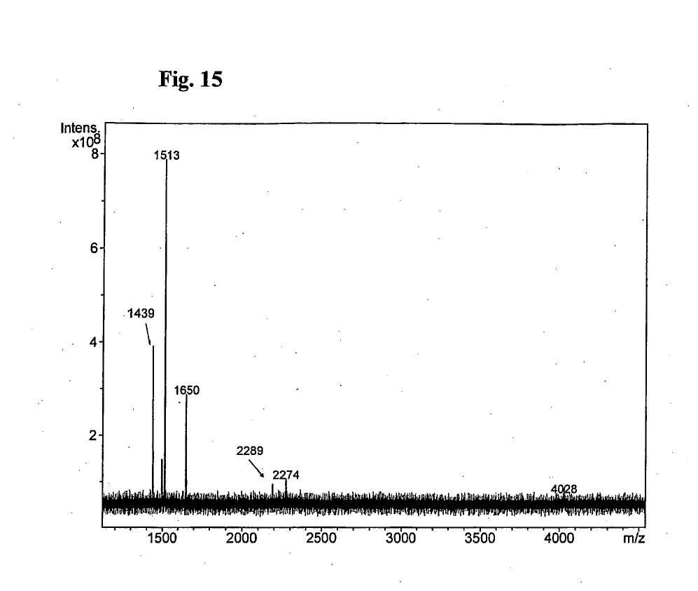

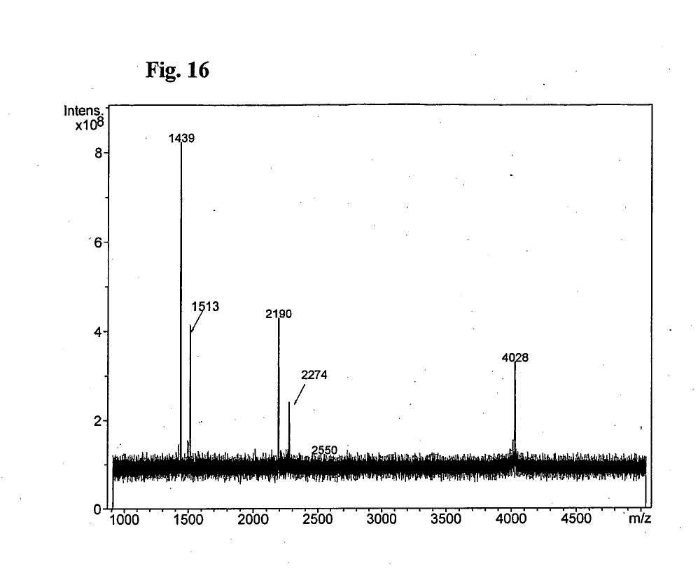

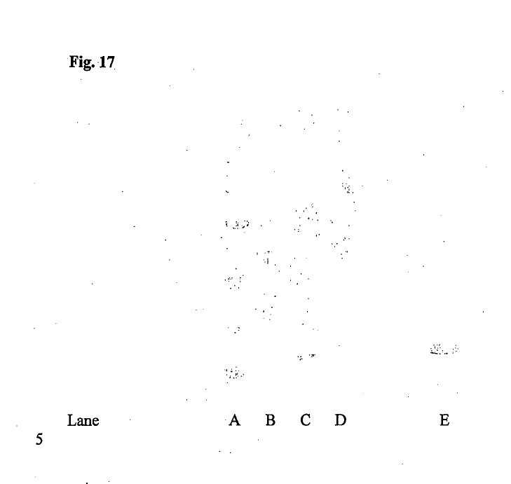

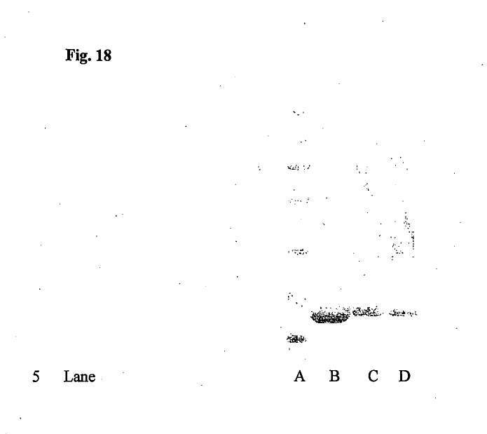

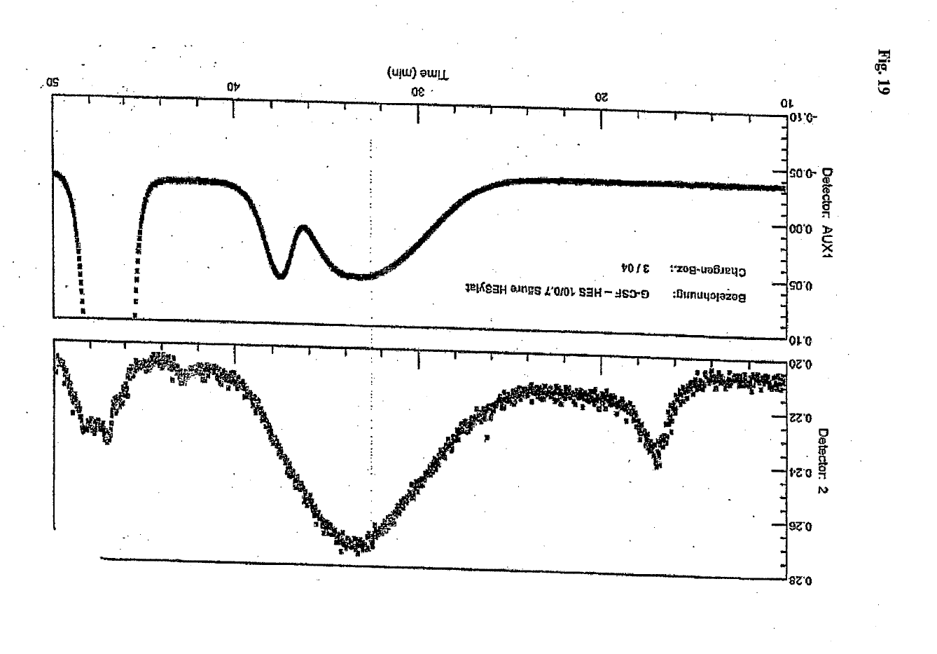

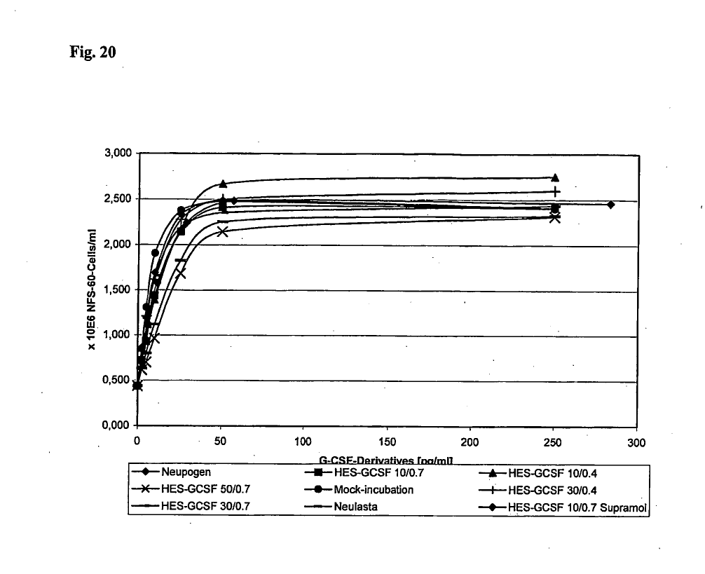

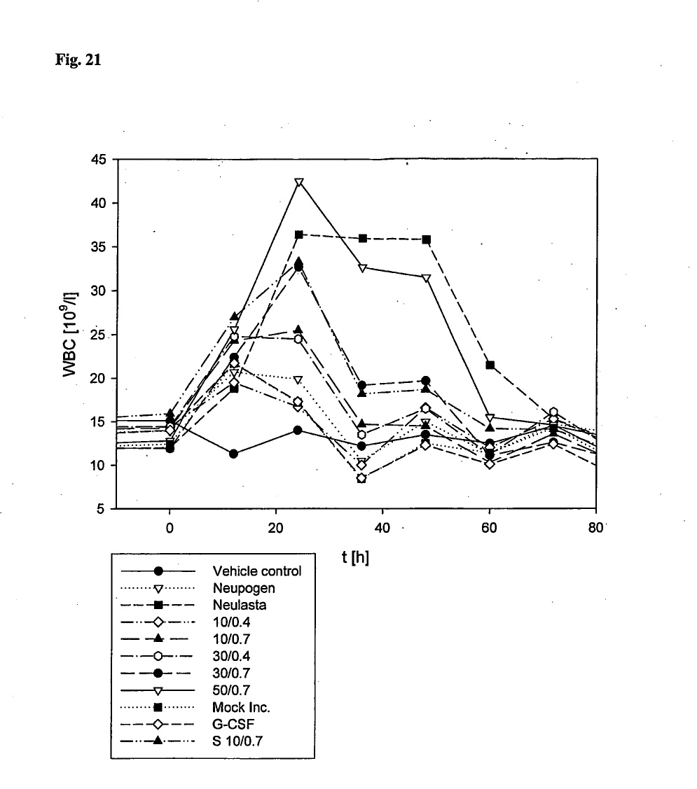

FIG. 1a shows an SDS page analysis of the HES-G-CSF conjugate, produced according to Example 2.1(a), Neupogen®. For gel electrophoresis, a XCell Sure Lock Mini Cell (Invitrogen GmbH, Karlsruhe, D) and a Consort E143 power supply (CONSORTnv, Turnhout, B) were employed. A 12% Bis-Tris gel together with a MOPS SDS running buffer at reducing conditions (both Invitrogen GmbH, Karlsruhe, D) were used according to the manufactures instruction.
Lane A: Protein marker SeeBlue®Plus2 (Invitrogen GmbH, Karlsruhe, D) Molecular weight marker from top to bottom: 188 kD, 98 kD, 62 kD, 49 kD, 38 kD, 28 kD, 17 kD, 14 kD, 6 kD, 3 kD.
Lane B: Crude product after conjugation of G-CSF (Neupogen®) with HES as described in Example 2.1(a).
Lane C: G-CSF starting material.

FIG. 1b

FIG. 1b shows an SDS page analysis of the HES-G-CSF conjugate, produced according to Example 2.1(a), Granocyte®. For gel electrophoresis, a XCell Sure Lock Mini Cell (Invitrogen GmbH, Karlsruhe, D) and a Consort E143 power supply (CONSORTnv, Turnhout, B) were employed. A 12% Bis-Tris gel together with a MOPS SDS running buffer at reducing conditions (both Invitrogen GmbH, Karlsruhe, D) were used according to the manufactures instruction.
Lane A: Protein marker SeeBlue®Plus2 (Invitrogen GmbH, Karlsruhe, D). Molecular weight marker from top to bottom: 188 kD, 98 kD, 62 kD, 49 kD, 38 kD, 28 kD, 17 kD, 14 kD, 6 kD, 3 kD.
Lane B: Crude product after conjugation of G-CSF (Granocyte®) with HES as described in Example 2.1(a).
Lane C: G-CSF starting material.

FIG. 2

FIG. 2 shows an SDS page analysis of the HES-G-CSF conjugate, produced according to Example 2.1(b), G-CSF from Strathmann Biotec AG, Hamburg, D. For gel electrophoresis a XCell Sure Lock Mini Cell (Invitrogen GmbH, Karlsruhe, D) and a Consort E143 power supply (CONSORTnv, Turnhout, B) were employed. A 12% Bis-Tris gel together with a MOPS SDS running buffer at reducing conditions (both Invitrogen GmbH, Karlsruhe, D) were used according to the manufactures instruction.
Lane A: Protein marker SeeBlue®Plus2 (Invitrogen GmbH, Karlsruhe, D). Molecular weight marker from top to bottom: 188 kD, 98 kD, 62 kD, 49 kD, 38 kD, 28 kD, 17 kD, 14 kD, 6 kD, 3 kD
Lane B: Crude product after conjugation of G-CSF with HES10/0.4 in 0.1M NaOAc buffer pH 5.0.
Lane C: Crude product after conjugation of G-CSF with HES10/0.7 in 0.1M NaOAc buffer pH 5.0.
Lane D: Crude product after conjugation of G-CSF with HES50/0.4 in 0.1M NaOAc buffer pH 5.0.
Lane E: Crude product after conjugation of G-CSF with HES50/0.7 in 0.1M NaOAc buffer pH 5.0.
Lane F: G-CSF starting material.

FIG. 3

FIG. 3 shows an SDS page analysis of the HES-G-CSF conjugates, produced according to Example 2.2, G-CSF from Strathmann Biotec AG, Hamburg, D. For gel electrophoresis a XCell Sure Lock Mini Cell (Invitrogen GmbH, Karlsruhe, D) and a Consort E143 power supply (CONSORTnv, Turnhout, B) were employed. A 12% Bis-Tris gel together with a MOPS SDS running buffer at reducing conditions (both Invitrogen GmbH, Karlsruhe, D) were used according to the manufactures instruction.
Lane A: Protein marker SeeBlue®Plus2 (Invitrogen GmbH, Karlsruhe, D). Molecular weight marker from top to bottom: 188 kD, 98 kD, 62 kD, 49 kD, 38 kD, 28 kD, 17 kD, 14 kD, 6 kD, 3 kD.
Lane B: Crude product after conjugation of G-CSF with oxidized HES10/0.7 in 0.1M NaOAc buffer pH 5.0.
Lane C: Crude product after conjugation of G-CSF with oxidized HES50/0.4 in 0.1M NaOAc buffer pH 5.0.
Lane D: Crude product after conjugation of G-CSF with oxidized HES50/0.7 in 0.1M NaOAc buffer pH 5.0.
Lane E: G-CSF starting material.

FIG. 4

FIG. 4 shows an SDS page analysis of the HES-G-CSF conjugates, produced according to Example 2.3, G-CSF is Neupogen® or Granocyte®. For gel electrophoresis a XCell Sure Lock Mini Cell (Invitrogen GmbH, Karlsruhe, D) and a Consort E143 power supply (CONSORTnv, Turnhout, B) were employed. A 12% Bis-Tris gel together with a MOPS SDS running buffer at reducing conditions (both Invitrogen GmbH, Karlsruhe, D) were used according to the manufactures instruction.
Lane A: Protein marker SeeBlue®Plus2 (Invitrogen GmbH, Karlsruhe, D). Molecular weight marker from top to bottom: 188 kD, 98 kD, 62 kD, 49 kD, 38 kD, 28 kD, 17 kD, 14 kD, 6 kD, 3 kD.
Lane B: Crude product (i-N) according to Example 2.3.
Lane C: Crude product (ii-N) according to Example 2.3.
Lane D: Crude product (iii-N) according to Example 2.3.
Lane E: Crude product (iv-N) according to Example 2.3.
Lane F: Crude product (i-G) according to Example 2.3.
Lane G: Crude product (ii-G) according to Example 2.3.
Lane H: Crude product (iii-G) according to Example 2.3.
Lane I: Crude product (iv-G) according to Example 2.3.
Lane J: Neupogen®.

FIG. 5

FIG. 5 shows an SDS page analysis of the HES-G-CSF conjugates, produced according to Example 2.4, G-CSF from Strathmann Biotec AG, Hamburg, D. For gel electrophoresis a XCell Sure Lock Mini Cell (Invitrogen GmbH, Karlsruhe, D) and a Consort E143 power supply (CONSORTnv, Turnhout, B) were employed. A 12% Bis-Tris gel together with a MOPS SDS running buffer at reducing conditions (both Invitrogen GmbH, Karlsruhe, D) were used according to the manufactures instruction.
Lane A: Protein marker SeeBlue®Plus2 (Invitrogen GmbH, Karlsruhe, D). Molecular weight marker from top to bottom: 188 kD, 98 kD, 62 kD, 49 kD, 38 kD, 28 kD, 17 kD, 14 kD, 6 kD, 3 kD.
Lane B: Crude product (vi) according to Example 2.4.
Lane C: Crude product (v) according to Example 2.4.
Lane D: G-CSF starting material.
Lane E: Protein marker SeeBlue®Plus2 (Invitrogen GmbH, Karlsruhe, D). Molecular weight marker from top to bottom: 188 kD, 98 kD, 62 kD, 49 kD, 38 kD, 28 kD, 17 kD, 14 kD, 6 kD, 3 kD.
Lane F: Crude product (ix) according to Example 2.4.
Lane G: Crude product (viii) according to Example 2.4.
Lane H: Crude product (vii) according to Example 2.4.
Lane I: G-CSF starting material.

FIG. 6

FIG. 6 shows an SDS page analysis of the HES-G-CSF conjugate, produced according to Example 2.5, G-CSF from Strathmann Biotec AG, Hamburg, D. For gel electrophoresis a XCell Sure Lock Mini Cell (Invitrogen GmbH, Karlsruhe, D) and a Consort E143 power supply (CONSORTnv, Turnhout, B) were employed. A 10% Bis-Tris gel together with a MOPS SDS running buffer at reducing conditions (both Invitrogen GmbH, Karlsruhe, D) were used according to the manufactures instruction.
Lane A: Protein marker SeeBlue®Plus2 (Invitrogen GmbH, Karlsruhe, D). Molecular weight marker from top to bottom: 188 kD, 98 kD, 62 kD, 49 kD, 38 kD, 28 kD, 17 kD, 14 kD, 6 kD, 3 kD.
Lane B: Crude product according to Example 2.5.
Lane C: G-CSF starting material.

FIG. 7

FIG. 7 shows an SDS page analysis of the HES-G-CSF conjugate, produced according to Example 3, G-CSF is Neupogen® or Granocyte®. For gel electrophoresis a XCell Sure Lock Mini Cell (Invitrogen GmbH, Karlsruhe, D) and a Consort E143 power supply (CONSORTnv, Turnhout, B) were employed. A 12% Bis-Tris gel together with a MOPS SDS running buffer at reducing conditions (both Invitrogen GmbH, Karlsruhe, D) were used according to the manufactures instruction.
Lane A: Protein marker SeeBlue®Plus2 (Invitrogen GmbH, Karlsruhe, D). Molecular weight marker from top to bottom: 188 kD, 98 kD, 62 kD, 49 kD, 38 kD, 28 kD, 17 kD, 14 kD, 6 kD, 3 kD
Lane B: Crude product (x) according to Example 3.
Lane C: Crude product (xi) according to Example 3.
Lane D: Crude product (xii) according to Example 3.
Lane E: Crude product (xiii) according to Example 3.
Lane F: Crude product (xiv) according to Example 3.
Lane G: Crude product (xv) according to Example 3.

FIG. 8

FIG. 8 shows the in vitro results of Example 6. In the diagram, the x axis shows the concentration in pg/ml, the y axis refers to the number of cells/100,000. In the diagram, the following abbreviations refer to

G-CSF/A32 G-CSF conjugate as prepared according to Example 2.5
G-CSF/A33 G-CSF starting material, used for the conjugate of Example 2.5
G-CSF/A57 non-modified Neulasta®
G-CSF/A58 non-modified Neupogen®
G-CSF/A60 G-CSF conjugate as prepared according to Example 4.2

FIG. 9

FIG. 9 shows the HPGPC chromatogram with regard to the crude conjugation reaction product according to Example 4.1. The following parameters were used in the HPGPC analysis:
Column: Superose 12 HR 10/30 300×10 mm I.D. (Pharmacia)
Eluent: 27.38 mM Na₂HPO₄; 12.62 mM NaH₂PO₄; 0.2 M NaCl; 0.005% NaN₃ in 111 of demineralized water
Flux: 0.24 ml/h
Detector 1: MALLS detector
Detector 2: UV (280 nm)
Detector 3: RI (Refractive Index detector)

A represents the result from detector 1, B represents the result from detector 2.

FIG. 10

FIG. 10 shows the HPGPC chromatogram with regard to the conjugation reaction product according to Example 4.1 where the content of the mixture regarding reaction by-products such as non-reacted oxo-HES and free N-hydroxysuccinimide as well as solvent was downgraded using a 10 kD ultrafiltration membrane in a cooling centrifuge.

The following parameters were used in the HPGPC analysis:
Column: Superose 12 HR 10/30 300×10 mm I.D. (Pharmacia)
Eluent: 27.38 mM Na₂HPO₄; 12.62 mM NaH₂PO₄; 0.2 M NaCl; 0.005% NaN₃ in 1 l of demineralized water
Flux: 0.24 ml/h
Detector 1: MALLS detector
Detector 2: UV (280 nm)
Detector 3: RI (Refractive Index detector)

A represents the result from detector 1, B represents the result from detector 2.

FIG. 11

The following parameters were used in the HPGPC analysis:
Column: Superose 12 HR 10/30 300×10 mm I.D. (Pharmacia)
Eluent: 27.38 mM Na₂HPO₄; 12.62 mM NaH₂PO₄; 0.2 M NaCl; 0.005% NaN₃ in 1 l of demineralized water
Flux: 0.24 ml/h
Detector 1: MALLS detector
Detector 2: UV (280 nm)
Detector 3: RI (Refractive Index detector)

A represents the result from detector 1, B represents the result from detector 2.

FIG. 12

The following parameters were used in the HPGPC analysis:
Column: Superose 12 HR 10/30 300×10 mm I.D. (Pharmacia)
Eluent: 27.38 mM Na₂HPO₄; 12.62 mM NaH₂PO₄; 0.2 M NaCl; 0.005% NaN₃ in 1 l of demineralized water
Flux: 0.24 ml/h
Detector 1: MALLS detector
Detector 2: UV (280 nm)
Detector 3: RI (Refractive index detector)

A represents the result from detector 1, B represents the result from detector 2.

FIG. 13

FIG. 13 shows the SDS-PAGE analysis of the flow-through and the eluate of HES-modified G-CSF (A32) after chromatography on DEAE-Sepharose CL-6B. 1.5% of the indicated fractions were desalted by ultrafiltration, dried in a SpeedVac and were applied onto a 12.5% polyacrylamide gel.

FIG. 14

FIG. 14 shows a MALDI/TOF spectrum of the G-CSF starting material (sample A33)

FIG. 15

FIG. 15 shows a MALDI/TOF spectrum of HES-modified G-CSF. (sample A32)

FIG. 16

FIG. 16 shows a MALDI/TOF spectrum of HES-modified G-CSF (sample A60)

FIG. 17

FIG. 17 shows the gel electrophoresis of the reaction mixtures of example 7.2(b). For gel electrophoresis a XCell Sure Lock Mini Cell (Invitrogen GmbH, Karlsruhe, D) and a Consort E143 power supply (CONSORTnv, Turnhout, B) were employed. A 12% Bis-Tris gel together with a MOPS SDS running buffer at reducing conditions (both Invitrogen GmbH, Karlsruhe, D) were used according to the manufactures instruction. The gel was stained with Roti-Blue (Carl Roth GmbH+Co.KG, Karlsruhe, D) according to the manufacturer's instruction.
Lane A: Protein marker Roti-Mark STANDARD (Carl Roth GmbH+Co.KG, Karlsruhe, D) Molecular weight marker from top to bottom: 200 KD, 119 KD, 66 KD, 43 KD, 29 KD, 20 KD, 14.3 KD
Lane B: Crude product after conjugation of hG-CSF with the HES derivative prepared in example 7.1(d)
Lane C: Crude product after conjugation of hG-CSF with the HES derivative prepared in example 7.1(b)
Lane D: Crude product after conjugation of hG-CSF with the HES derivative prepared in example 7.1(j)
Lane E: Reaction control: HES 50/07

FIG. 18

FIG. 18 shows the gel electrophoresis of the reaction mixtures of example 7.2(d). For gel electrophoresis a XCell Sure Lock Mini Cell (Invitrogen GmbH, Karlsruhe, D) and a Consort E143 power supply (CONSORTnv, Turnhout, B) were employed. A 12% Bis-Tris gel together with a MOPS SDS running buffer at reducing conditions (both Invitrogen GmbH, Karlsruhe, D) were used according to the manufactures instruction. The gel was stained with Roti-Blue (Carl Roth GmbH+Co.KG, Karlsruhe, D) according to the manufacturer's instruction.
Lane A: Protein marker Roti-Mark STANDARD (Carl Roth GmbH+Co.KG, Karlsruhe, D) Molecular weight marker from top to bottom: 200 KD, 119 KD, 66 KD, 43 KD, 29 KD, 20 KD, 14.3 KD.
Lane B: hG-CSF after buffer exchange as described in Example 7.2(c).
Lane C: Crude product after conjugation of hG-CSF with the HES derivative prepared as described in Example 7.1(f).
Lane D: Crude product after conjugation of hG-CSF with the HES derivative prepared as described in Example 7.1(h).

FIG. 19

FIG. 19 shows the HPGPC chromatogram with regard to the conjugation reaction product according to Example 7.3 (MALLS detector: upper chart; UV detector: lower chart). The following parameters were used in the HPGPC analysis:
Column: Superose 12 HR 10/30 300×10 mm I.D. (Pharmacia)
Eluent: 27.38 mM Na₂HPO₄; 12.62 mM NaH₂PO₄; 0.2 M NaCl; 0.005% NaN₃ in 1 l of demineralized water
Flux: 0.24 ml/h
Detector 1: MALLS detector
Detector 2: UV (280 nm)
Detector 3: RI (Refractive Index detector)

FIG. 20

FIG. 20 shows the results of the mitogenicity assay of example 7.4. The Y axis indicates number of NFS-60-Cells/ml and the X-axis the concentration in pg/ml.

FIG. 21

FIG. 21 shows the results of the in vivo assay of example 7.5.

### EXAMPLES

### Example 1

### Synthesis of Aldehyde Functionalized Hydroxyethyl Starch

### Example 1.1(a)

### Synthesis by Periodate Oxidation of Hydroxyethyl Starch Selectively Oxidized at its Reducing End and Incubation at 0° C.

100 mg of Oxo-HES10/0.4 (MW=10 kD, DS=0.4, prepared by Supramol Parenteral Colloids GmbH, Rosbach-Rodheim, D; according to DE 196 28 705 A1, were dissolved in 5 ml 20 mM sodium phosphate buffer, pH 7.2 and cooled to 0° C. 21.4 mg sodium periodate (Fluka, Sigma-Aldrich Chemie GmbH, Taufkirchen, D) were dissolved in 5 ml of the same buffer and cooled to 0° C. Both solutions were mixed and after incubation for 10 min at 0° C., 0.73 ml glycerol were added and the reaction mixture was incubated at 21° C. for 10 min. The reaction mixture was dialysed for 24 h against water (SnakeSkin dialysis tubing, 3.5 kD cut off, Perbio Sciences Deutschland GmbH, Bonn, D) and lyophilized.

### Example 1.1(b)

### Synthesis by Periodate Oxidation of Hydroxyethyl Starch Selectively Oxidized at its Reducing End and Incubation at 21° C.

100 mg of Oxo-HES10/0.4 (MW=10 kD, DS=0.4, prepared by Supramol Parenteral Colloids GmbH, Rosbach-Rodheim, D; according to DE 196 28 705 A1) were dissolved in 5 ml 20 mM sodium phosphate buffer, pH 7.2. 21.4 mg sodium periodate (Fluka, Sigma-Aldrich Chemie GmbH, Taufkirchen, D) were dissolved in 5 ml of the same buffer. Both solutions were mixed and after incubation for 10 min at 21° C. 0.73 ml glycerol were added and the reaction mixture was incubated at 21° C. for 10 min. The reaction mixture was dialysed for 24 h against water (SnakeSkin dialysis tubing, 3.5 kD cut off, Perbio Sciences Deutschland GmbH, Bonn, D) and lyophilized.

### Example 1.2(a)

### Synthesis of Aldehyde Functionalized Hydroxyethyl Starch by Periodate Oxidation of Hydroxyethyl Starch with Non-Oxidized Reducing End and Incubation at 0° C.

100 mg of HES10/0.4 (MW=10 kD, DS=0.4, Supramol Parenteral Colloids GmbH, Rosbach-Rodheim, D) were dissolved in 5 ml 20 mM sodium phosphate buffer, pH 7.2 and cooled to 0° C. 21.4 mg sodium periodate (Fluka, Sigma-Aldrich Chemie GmbH, Taufkirchen, D) were dissolved in 5 ml of the same buffer and cooled to 0° C. Both solutions were mixed and after incubation for 10 min at 0° C. 0.73 ml glycerol were added and the reaction mixture was incubated at 21° C. for 10 min. The reaction mixture was dialysed for 24 h against water (SnakeSkin dialysis tubing, 3.5 kD cut off, Perbio Sciences Deutschland GmbH, Bonn, D) and lyophilized.

### Example 1.2(b)

### Synthesis of Aldehyde Functionalized Hydroxyethyl Starch by Periodate Oxidation of Hydroxyethyl Starch with Non-Oxidized Reducing End and Incubation at 21° C.

100 mg of HES10/0.4 (MW=10 kD, DS=0.4, prepared by Supramol Parenteral Colloids GmbH, Rosbach-Rodheim, D) were dissolved in 5 ml 20 mM sodium phosphate buffer, pH 7.2. 21.4 mg sodium periodate (Fluka, Sigma-Aldrich Chemie GmbH, Taufkirchen, D) were dissolved in 5 ml of the same buffer. Both solutions were mixed and after incubation for 10 min at 21° C. 0.73 ml glycerol were added and the reaction mixture was incubated at 21° C. for 10 min. The reaction mixture was dialysed for 24 h against water (SnakeSkin dialysis tubing, 3.5 kD cut off, Perbio Sciences Deutschland GmbH, Bonn, D) and lyophilized.

### Example 1.3

### Synthesis of Aldehyde Functionalized Hydroxyethyl Starch from Amino Functionalized Hydroxyethyl Starch and Formylbenzoic Acid

Oxo-HES10/0.4 (MW=10 kD, DS=0.4) was prepared by Supramol Parenteral Colloids GmbH, Rosbach-Rodheim, D; according to DE 196 28 705 A1.

5.1 g (0.51 mmol) of oxo-HES10/0.4 were dissolved in 15 ml anhydrous dimethyl sulfoxide (DMSO, Fluka, Sigma-Aldrich Chemie GmbH, Taufkirchen, D)) and added dropwise under nitrogen to a solution of 5.1 ml (51 mmol) 1,4-diaminobutane in 10 ml anhydrous dimethyl sulfoxide and stirred at 40° C. for 19 h. The reaction mixture was added to a mixture of 80 ml ethanol and 80 ml acetone. The resulting precipitate was separated by centrifugation, washed with a mixture of 20 ml ethanol and 20 ml acetone and re-dissolved in 80 ml water. The solution was dialyzed for 4 days against water (SnakeSkin dialysis tubing, 3.5 kD cut off, Perbio Science Deutschland GmbH, Bonn, D) and subsequently lyophilized. The yield was 67% (3.4 g) amino-HES10/0.4.

150 mg 4-formylbenzoic acid and 230 mg 1-hydroxy-1H-benzotriazole (both Aldrich, Sigma-Aldrich Chemie GmbH, Taufkirchen, D) were dissolved in 10 ml N,N-dimethylformamide (Peptide synthesis grade, Biosolve, Valkenswaard, NL) and 204 μl N,N′-diisopropylcarbodiimide were added. After incubation at 21° C. for 30 min, 1 g of the amino-HES10/0.4 were added. After shaking for 19 h at 22° C., the reaction mixture was added to 84 mL of an ice-cold 1:1 mixture of acetone and ethanol (v/v). The precipitated product was collected by centrifugation at 4° C., re-dissolved in 50 m water, dialysed for 2 d against water (SnakeSkin dialysis tubing, 3.5 kD cut off, Perbio Sciences Deutschland GmbH, Bonn, D) and lyophilized.

### Example 1.4

### Synthesis of Aldehyde Functionalized Hydroxyethyl Starch from Hydroxyethyl Starch and Formylbenzoic Acid

Oxo-HES10/0.7 (MW=10 kD, DS=0.7) was prepared by Supramol Parenteral Colloids GmbH, Rosbach-Rodheim, D; according to DE 196 28 705 A1.

83 mg of 4-formylbenzoic acid and 180 mg 1-hydroxy-1H-benzotriazole (both Aldrich, Sigma-Aldrich Chemie GmbH, Taufkirchen, D) were dissolved in 5 mL N,N-dimethylformamide (DMF, Peptide synthesis grade, Biosolve, Valkenswaard, NL) and 78 μl N,N′-diisopropylcarbodiimide were added. After incubation at 21° C. for 30 min, 0.5 g of oxo-HES10/0.7 were added. After shaking for 19 h at 22° C., the reaction mixture was added to 37.5 ml of an ice-cold 1:1 mixture of acetone and ethanol (v/v). The precipitated product was collected by centrifugation at 4° C., re-dissolved in a mixture of 2.5 ml water and 2.5 ml DMF and precipitated again as described above. The reaction product was collected by centrifugation as described, re-dissolved in 10 ml water, dialysed for 2 d against water (SnakeSkin dialysis tubing, 3.5 kD cut off, Perbio Sciences Deutschland GmbH, Bonn, D) and lyophilized.

### Example 1.5

### Synthesis of Aldehyde Functionalized Hydroxyethyl Starch from Hydroxyethyl Starch and Formylbenzoic Acid

HES10/0.7 (MW=10 kD, DS=0.7) was prepared by Supramol Parenteral Colloids GmbH, Rosbach-Rodheim, D.

50 mg 4-formylbenzoic acid and 108 mg 1-hydroxy-1H-benzotriazole (both Aldrich, Sigma-Aldrich Chemie GmbH, Taufkirchen, D) were dissolved in 3 ml N,N-dimethylformamide (Peptide synthesis grade, Biosolve, Valkenswaard, NL) and 47 μl N,N′-diisopropylcarbodiimide were added. After incubation at 21° C. for 30 min, 0.3 g of HES10/0.7 were added. After shaking for 19 h at 22° C., the reaction mixture was added to 23 ml of an ice-cold 1:1 mixture of acetone and ethanol (v/v). The precipitated product was collected by centrifugation at 4° C., re-dissolved in a mixture of 1.5 ml water and 1.5 ml DMF and precipitated again as described above. The reaction product was collected by centrifugation as described, re-dissolved in 10 ml water, dialysed for 2 d against water (SnakeSkin dialysis tubing, 3.5 kD cut off, Perbio Sciences Deutschland GmbH, Bonn, D) and lyophilized.

### Example 1.6

### Synthesis of Aldehyde Functionalized Hydroxyethyl Starch from Amino Functionalized Hydroxyethyl Starch and Formylbenzoic Acid Pentafluorophenyl Ester

Oxo-HES10/0.7 (MW=10 kD, DS=0.7) was prepared by Supramol Parenteral Colloids GmbH, Rosbach-Rodheim, D; according to DE 196 28 705 A1.

6.0 g (0.6 mmol) of oxo-HES10/0.7 were dissolved in 20 ml anhydrous dimethyl sulfoxide (DMSO, Fluka, Sigma-Aldrich Chemie GmbH, Taufkirchen, D)) and added dropwise under nitrogen to a solution of 6 ml (60 mmol) 1,4-diaminobutane in 11 ml anhydrous dimethyl sulfoxide and stirred at 40° C. for 19 h. The reaction mixture was added to a mixture of 80 ml ethanol and 80 ml acetone. The resulting precipitate was separated by centrifugation, washed with a mixture of 20 ml ethanol and 20 ml acetone and re-dissolved in 80 ml water. The solution was dialyzed for 4 days against water (SnakeSkin dialysis tubing, 3.5 kD cut off, Perbio Science Deutschland GmbH, Bonn, D) and subsequently lyophilized. The yield was 52% (3.15 g) amino-HES10/0.7.

4-formylbenzoic acid pentafluorophenyl ester was synthesized as described in J. S. Lindsey at al., Tetrahedron 50 (1994) pp. 8941-68, especially p. 8956. 50 mg of amino-HES10/0.7 were dissolved in 0.5 ml N,N-dimethylformamide (Peptide synthesis grade, Biosolve, Valkenswaard, NL) and 15.3 mg 4-formylbenzoic acid pentafluorophenylester were added. After shaking for 22 h at 22° C., the reaction mixture was added to 3.5 ml of ice-cold 2-propanol. The precipitated product was collected by centrifugation at 4° C., washed with 4 ml ice-cold 2-propanol, re-dissolved in 50 ml water, dialysed for 2 d against water (SnakeSkin dialysis tubing, 3.5 kD cut off, Perbio Sciences Deutschland GmbH, Bonn, D) and lyophilized.

### Example 1.7

### Synthesis of Aldehyde Functionalized Hydroxyethyl Starch from Hydroxyethyl Starch and Formylbenzoic Acid Pentafluorophenyl Ester

Oxo-HES10/0.7 (MW=10 kD, DS=0.7) was prepared by Supramol Parenteral Colloids GmbH, Rosbach-Rodheim, D; according to DE 196 28 705 A1.

4-formylbenzoic acid pentafluorophenyl ester was synthesized as described in J. S. Lindsey at al., Tetrahedron 50 (1994) pp. 8941-68, especially p. 8956. 200 mg oxo-HES10/0.7 were dissolved in 2 ml N,N-dimethylformamide (Peptide synthesis grade, Biosolve, Valkenswaard, NL) and 61.2 mg 4-formylbenzoic acid pentafluorophenyl ester were added. After shaking for 22 h at 22° C., the reaction mixture was added to 15 mL of ice-cold 1:1 mixture of acetone and ethanol (v/v). The precipitated product was collected by centrifugation at 4° C., re-dissolved in a mixture of 1.4 ml water and 0.7 ml DMF and precipitated again as described above. The reaction product was collected by centrifugation as described, re-dissolved in 10 ml water, dialysed for 2 d against water (SnakeSkin dialysis tubing, 3.5 kD cut off, Perbio Sciences Deutschland GmbH, Bonn, D) and lyophilized.

### Example 1.8

### Synthesis of aldehyde functionalized hydroxyethyl starch from amino functionalized hydroxyethyl starch and 4-(4-formyl-3,5-dimethoxyphenoxy)butyric acid

Oxo-HES10/0.4 (MW=10 kD, DS=0.4) was prepared by Supramol Parenteral Colloids GmbH, Rosbach-Rodheim, D; according to DE 196 28 705 A1.

5.1 g (0.51 mmol) of oxo-HES10/0.4 were dissolved in 15 ml anhydrous dimethyl sulfoxide (DMSO, Fluka, Sigma-Aldrich Chemie GmbH, Taufkirchen, D)) and added dropwise under nitrogen to a solution of 5.1 ml (51 mmol) 1,4-diaminobutane in 10 ml anhydrous dimethyl sulfoxide and stirred at 40° C. for 19 h. The reaction mixture was added to a mixture of 80 ml ethanol and 80 ml acetone. The resulting precipitate was separated by centrifugation, washed with a mixture of 20 ml ethanol and 20 ml acetone and re-dissolved in 80 ml water. The solution was dialyzed for 4 days against water (SnakeSkin dialysis tubing, 3.5 kD cut off, Perbio Science Deutschland GmbH, Bonn, D) and subsequently lyophilized. The yield was 67% (3.4 g) amino-HES10/0.4.

80.5 mg 4-(4-formyl-3,5-dimethoxyphenoxy)butyric acid (Calbiochem-Novabiochem, Läufelfingen, CH) and 61 mg 1-hydroxy-1H-benzotriazole (Aldrich, Sigma-Aldrich Chemie GmbH, Taufkirchen, D) were dissolved in 3 ml N,N-dimethylformamide (Peptide synthesis grade, Biosolve, Valkenswaard, NL) and 45.4 μl N,N′-diisopropylcarbodiimide were added. After incubation at 21° C. for 30 min, 0.3 g of amino-HES10/0.4 were added. After shaking for 22 h at 22° C., the reaction mixture was added to 23 ml of ice-cold 1:1 mixture of acetone and ethanol (v/v). The precipitated product was collected by centrifugation at 4° C., re-dissolved in a mixture of 2 ml water and 1 ml DMF and precipitated again as described above. The reaction product was collected by centrifugation as described, re-dissolved in 10 ml water, dialysed for 1 d against water (SnakeSkin dialysis tubing, 3.5 kD cut off, Perbio Sciences Deutschland GmbH, Bonn, D) and lyophilized.

### Example 2

### Synthesis of G-CSF Conjugates by Reductive Amination

### Example 2.1(a)

### Synthesis of G-CSF-Conjugates by Reductive Amination with Hydroxyethyl Starch with Non-Oxidized Reducing End at pH=7.4

### Comparative Example

In Example 2.1, it was tried to use the synthesis method of WO 03/074087 (example 12, page 22-23) for the production of a HES-G-CSF conjugate.

To 3.33 μl of an aqueous solution of G-CSF (Neupogen® from Amgen, München, D, or Granocyte® from Aventis Pharma AG, Zürich, CH, respectively, 3 mg/ml) in 0.1 M sodium phosphate buffer with pH 7.4, 3.33 μl of a solution of HES10/0.4 (MW=10 kD, DS=0.4, Supramol Parenteral Colloids GmbH, Rosbach-Rodheim, D, 79 mg/ml) in the same buffer were added. To this mixture, 3.33 μl of a 60 mM solution of sodium cyanoborohydride in the same buffer was added, and the resulting mixture was incubated for 4 h at 22° C. Subsequently, another 3.33 μl of the freshly prepared 60 mM sodium cyanoborohydride solution were added. During the incubation time of 30 h, altogether 5 portions of 3.33 μl of a freshly prepared 60 mM sodium cyanoborohydride solution were added. The reaction mixture was analysed by gel electrophoresis. No reaction was observed.

### Example 2.1(b)

### Synthesis of G-CSF-Conjugates by Reductive Amination with Hydroxyethyl Starch with Non-Oxidized Reducing End at a pH of from 5.0 to 9.2

### Comparative Example

To 3.33 μL of an aqueous solution of G-CSF (G-CSF from Strathmann Biotec AG, Hamburg, D, 3 mg/mL) in a given buffer, 3.33 μl of a HES solution (300 mg/ml) in the same buffer were added. The mixture was cooled to 4° C., and 3.33 μl of a 60 mM solution of sodium cyanoborohydride in the same buffer at 4° C. were added, and the resulting mixture was incubated for 20 h at 4° C.

The following HES preparations and buffer were employed:

a) Buffer: 0.1 M sodium acetate buffer pH 5.0
HES10/0.4 (MW=10 kD, DS=0.4, Supramol Parenteral Colloids GmbH, Rosbach-Rodheim, D)
HES10/0.7 (MW=10 kD, DS=0.7, Supramol Parenteral Colloids GmbH, Rosbach-Rodheim, D)
HES50/0.4 (MW=50 kD, DS=0.4, Supramol Parenteral Colloids GmbH, Rosbach-Rodheim, D)
HES50/0.7 (MW=50 kD, DS=0.7, Supramol Parenteral Colloids GmbH, Rosbach-Rodheim, D)
   b) Buffer: 0.1 M sodium phosphate buffer pH 7.2
HES10/0.7 (MW=10 kD, DS=0.7, Supramol Parenteral Colloids GmbH, Rosbach-Rodheim, D)
   c) Buffer: 0.1 M sodium borate buffer pH 8.3
HES10/0.7 (MW=10 kD, DS=0.7, Supramol Parenteral Colloids GmbH, Rosbach-Rodheim, D).
   d) Buffer: 0.2 M potassium borate buffer pH 9.2
HES10/0.7 (MW=10 kD, DS=0.7, Supramol Parenteral Colloids GmbH, Rosbach-Rodheim, D)

Each reaction mixture was analysed by gel electrophoresis. No or negligible conjugation was observed (gel scans for reactions b) to d) not shown).

### Example 2.2

### Synthesis of G-CSF-Conjugates by Reductive Amination with Hydroxyethyl Starch with Oxidized Reducing End at a pH of 5.0 from 9.2

### Comparative Example

To 3.33 μL of an aqueous solution of G-CSF (G-CSF from Strathmann Biotec AG, Hamburg, D, 3 mg/ml) in a given buffer, 3.33 μl of a solution of oxo-HES (300 mg/ml) in the same buffer were added. The mixture was cooled to 4° C., and 3.33 μl of a 60 mM solution of sodium cyanoborohydride in the same buffer at 4° C. were added, and the mixture was incubated for 17 h at 4° C.

The following HES preparations and buffer were employed:

a) Buffer: 0.1 M sodium acetate buffer pH 5.0
oxo-HES10/0.7 (MW=10 kD, DS=0.7, Supramol Parenteral Colloids GmbH, Rosbach-Rodheim, D)
oxo-HES50/0.4 (MW=50 kD, DS=0.4, Supramol Parenteral Colloids GmbH, Rosbach-Rodheim, D)
oxo-HES50/0.7 (W=50 kD, DS=0.7, Supramol Parenteral Colloids GmbH, Rosbach-Rodheim, D)
   b) Buffer: 0.1 M sodium phosphate buffer pH 7.2
HES10/0.7 (MW=10 kD, DS=0.7, Supramol Parenteral Colloids GmbH, Rosbach-Rodheim, D)
   c) Buffer: 0.1 M sodium borate buffer pH 8.3
HES10/0.7 (MW=10 kD, DS=0.7, Supramol Parenteral Colloids GmbH, Rosbach-Rodheim, D)
   d) Buffer: 0.2 M potassium borate buffer pH 9.2
HES10/0.7 (MW=10 kD, DS=0.7, Supramol Parenteral Colloids GmbH, Rosbach-Rodheim, D)

Each reaction mixture was analysed by gel electrophoresis. No or negligible conjugation was observed (gel scans for reactions b) to d) not shown).

Oxidation of HES10/0.4 (MW=10 kD, DS=0.4) was carried out by Supramol Parenteral Colloids GmbH, Rosbach-Rodheim, D; according to DE 196 28 705 A1.

### Example 2.3

### Synthesis of G-CSF-Conjugates by Reductive Amination with Aldehyde Functionalized Hydroxyethyl Starch Synthesized by Periodate Oxidation

To 3.33 μl of an aqueous solution of G-CSF (Granocyte® from Aventis Pharma AG, Zürich, CH, and Neupogen® from Amgen, München, D, respectively, 3 mg/mL) in 0.1 M sodium acetate buffer pH 5.0, 3.33 μl of a solution of an aldehydro-HES (79 mg/mL) in the same buffer were added. To the mixture 3.33 μL of a 60 mM solution of sodium cyanoborohydride in the same buffer were added and the mixture was incubated for 25 h at 21° C. The reaction mixture was analysed by gel electrophoresis.

The following aldehyde functionalized HES conjugates were employed:
(i-N) prepared with Neupogen® according to Example 1.1(a) hereinabove;
(ii-N) prepared with Neupogen® according to Example 1.1(b) hereinabove;
(iii-N) prepared with Neupogen® according to Example 1.2(a) hereinabove;
(iv-N) prepared with Neupogen® according to Example 1.2(b) hereinabove;
(i-G) prepared with Granocyte® according to Example 1.1(a) hereinabove;
(ii-G) prepared with Granocyte® according to Example 1.1(b) hereinabove;
(iii-G) prepared with Granocyte® according to Example 1.2(a) hereinabove;
(iv-G) prepared with Granocyte® according to Example 1.2(b) hereinabove.

### Example 2.4

### Synthesis of G-CSF-Conjugates by Reductive Amination with Aldehyde Functionalized Hydroxyethyl Starch Synthesized by Conjugation of Hydroxyethyl Starch to a Formyl-Carboxylic Acid

To 3.33 μl of an aqueous solution of G-CSF (G-CSF from Strathmann Biotec AG, Hamburg, D, 3 mg/ml) in 0.1 M sodium acetate buffer pH 5.0, 3.33 μl of a solution of an aldehydro-HES (118.5 mg/mL) in the same buffer were added and cooled to 4° C. To the mixture 3.33 μl of a 60 mM solution of sodium cyanoborohydride in the same buffer at 4° C. were added and the mixture was incubated for 17 h at 4° C. The reaction mixture was analysed by gel electrophoresis.

The following aldehyde functionalized HES conjugates were employed:
(v) prepared according to Example 1.4 hereinabove;
(vi) prepared according to Example 1.5 hereinabove;
(vii) prepared according to Example 1.6 hereinabove;
(viii) prepared according to Example 1.7 hereinabove;
(ix) prepared according to Example 1.8 hereinabove.

### Example 2.5

### Synthesis of G-CSF-Conjugates by Reductive Amination with Aldehyde Functionalized Hydroxyethyl Starch Synthesized by Conjugation of Hydroxyethyl Starch to Formyl-Carboxylic Acid

To 2.5 ml of an aqueous solution of G-CSF (G-CSF from Strathmann Biotec AG, Hamburg, D, 2.27 mg/ml) in 0.1 M sodium acetate buffer pH 5.0, 136 mg aldehydro-HES10/0.4, prepared as described in Example 1.3 hereinabove, were added, and the solution was cooled to 0° C. To the mixture 2.5 ml of an ice-cold 40 mM solution of sodium cyanoborohydride in the same buffer were added and the mixture was incubated for 17 h at 4° C. The reaction mixture was analysed by gel electrophoresis.

### Example 3

### Synthesis of G-CSF Conjugates by SH Alkylation

Oxo-HES10/0.7 (MW=10 kD, DS=0.7) was prepared by Supramol Parenteral Colloids GmbH, Rosbach-Rodheim, D; according to DE 196 28 705 A1.

6.0 g (0.6 mmol) of oxo-HES10/0.7 were dissolved in 20 ml anhydrous dimethyl sulfoxide (DMSO, Fluka, Sigma-Aldrich Chemie GmbH, Taufkirchen, D)) and added dropwise under nitrogen to a solution of 6 ml (60 mmol) 1,4-diaminobutane in 11 ml anhydrous dimethyl sulfoxide and stirred at 40° C. for 19 h. The reaction mixture was added to a mixture of 80 ml ethanol and 80 ml acetone. The resulting precipitate was separated by centrifugation, washed with a mixture of 20 ml ethanol and 20 ml acetone and re-dissolved in 80 ml water. The solution was dialyzed for 4 days against water (SnakeSkin dialysis tubing, 3.5 kD cut off, Perbio Science Deutschland GmbH, Bonn, D) and subsequently lyophilized. The yield was 52% (3.15 g) amino-HES10/0.7.

To 132 μg amino-HES10/0.7, dissolved in 100 μl sodium phosphate buffer (0.1 M, 0.15 M NaCl, 50 mM EDTA, pH 7.2), 10 μl of a solution of 17.5 mg/ml N-alpha(maleimidoacetoxy)succinimide ester (AMAS) in dry DMSO (both Fluka, Sigma-Aldrich Chemie GmbH, Taufkirchen, D) were added, and the clear solution was incubated for 80 min at 25° C. and subsequently for 20 min at 40° C. The excess of AMAS was removed by centrifugal filtration with a VIVASPIN 0.5 ml concentrator, 5 KD MWCO (VIVASCIENCE, Hannover, D) at 13,000 rpm, washed 2 times for 30 min with 450 μl of the phosphate buffer and once with 450 μl of a buffer B. To the residual solution, 10 μg G-CSF (Neupogen® from Amgen, München, D, and Granocyte® from Aventis Pharma AG, Zürich, CH, respectively, 3 μg/μl in phosphate buffer) were added, and the mixture was incubated for 16 h at 25° C. The reaction mixture was analysed by gel electrophoresis after concentration in vacuo.

The following methods were chosen:
(x) G-CSF (Granocyte®) employing sodium phosphate buffer (0.1 M, 0.15 M NaCl, 50 mM EDTA, pH 7.2) as buffer B.
(xi) G-CSF (Neupogen®) employing sodium phosphate buffer (0.1 M, 0.15 M NaCl, 50 mM EDTA, pH 7.2) as buffer B.
(xii) G-CSF (Granocyte®) employing a 1:1 (v/v) mixture of sodium phosphate buffer (0.1 M, 0.15 M NaCl, 50 mM EDTA, pH 7.2) and 8 M urea, 1% SDS, pH 7.4 as buffer B.
(xiii) G-CSF (Neupogen®) employing a 1:1 (v/v) mixture of sodium phosphate buffer (0.1 M, 0.15 M NaCl, 50 mM EDTA, pH 7.2) and 8 M urea, 1% SDS, pH 7.4 as buffer B.
(xiv) G-CSF (Granocyte®) employing 8 M urea, 1% SDS, pH 7.4 as buffer B.
(xv) G-CSF (Neupogen®) employing 8 M urea, 1% SDS, pH 7.4 as buffer B.

### Example 4

### Synthesis of G-CSF Conjugates by Reaction of Hydroxyethyl Starch Having a Reactive Ester Group with G-CSF

### Example 4.1

Oxo-HES10/0.4 (MW=10,559 D, DS=0.4) was prepared by Supramol Parenteral Colloids GmbH, Rosbach-Rodheim, D; according to DE 196 28 705 A1. The degree of oxidation of oxo-HES was 95%

66 mg of oxo-HES10/0.4 were dissolved in 0.5 ml anhydrous DMF. To this solution, 3.4 mg of N,N′-disuccinimidyl carbonate were added, and the mixture was stirred for 2 h at room temperature. The resulting solution had a reactive HES concentration of 13 percent by weight.

A solution of G-CSF (Strathmann Biotec AG, Hamburg, D), having a concentration of about 0.5 mg G-CSF/ml, was concentrated to a concentration of 10 mg/ml by ultracentrifugation at a cut-off of 10 kD using a cooling centrifuge. To 0.5 ml of this concentrated G-CSF solution, 180 μL of a sodium bicarbonate solution were added. Subsequently, 3 portions (100 μl each) of the reactive HES solution were added dropwise to the protein solution, until, after about 30 min., the reaction had come to an end. Thus, the overall molar ratio of reactive HES:G-CSF was 20:1. Then, the pH of the mixture was adjusted to 4.0 using 0.1 N HCl.

A HPGPC analysis (High-Performance Gel Permeation Chromatography) gave a yield of about 70%. This result is shown in FIG. 9.

The mixture could be stored at 4° C. at a pH of 4.0 for 4 d and, according to HPGPC analyses, remained stable, i.e. unchanged.

Downgrading the content of the mixture regarding reaction by-products such as non-reacted oxo-HES and free N-hydroxysuccinimide as well as solvent using a 10 kD ultrafiltration membrane in a cooling centrifuge was possible without difficulties. The results of this downgrading experiment is shown in FIG. 10.

### Example 4.2

Oxo-HES10/0.4 (MW=10,559 D, DS=0.4) was prepared by Supramol Parenteral Colloids GmbH, Rosbach-Rodheim, D; according to DE 196 28 705 A1. The degree of oxidation of oxo-HES was 95%

400 mg of oxo-HES10/0.4 were dissolved in 1 ml anhydrous DMF. To this solution, 21 mg of N,N′-disuccinimidyl carbonate were added, and the mixture was stirred for 2 h at room temperature. The resulting solution had a reactive HES concentration of 40 percent by weight.

A solution of G-CSF (Strathmann Biotec AG, Hamburg, D), having a concentration of about 0.5 mg G-CSF/ml, was concentrated to a concentration of 10 mg/ml by ultracentrifugation at a cut-off of 10 kD using a cooling centrifuge.

To 0.5 ml of this concentrated G-CSF solution, 180 μl of a sodium bicarbonate solution were added. Subsequently, 3 portions (100 μl each) of the reactive HES solution were added dropwise to the protein solution, until, after about 30 min., the reaction had come to an end. Thus, the overall molar ratio of reactive HES G-CSF was about 50:1. Then, the pH of the mixture was adjusted to 4.0 using 0.1 N HCl.

A HPGPC analysis (High-Performance Gel Permeation Chromatography) gave a yield of more than 95%. No non-reacted G-CSF could be detected. This result is shown in FIG. 11.

The mixture was purified using ultrafiltration technology without difficulties. The results of these downgrading is shown in FIG. 12.

### Example 5

### 5.1. Purification

Purified G-CSF having essentially the same characteristics as the commercial product Neupogen® (Amgen) was obtained and one aliquot was kept unmodified as a control.

### 5.2 Synthesis of Conjugates of HES and G-CSF

Conjugates were synthesized essentially as described in Example 4.2, but with Oxo-HES50/0.7 (sample code A60), or as described in Example 2.5 (sample code A32), and used for further buffer exchange and purification.

### 5.3 Buffer Exchange of G-CSF and HES-Modified G-CSF Samples Before Purification by Anion-Exchange Chromatography

HES-modified G-CSF samples or unmodified G-CSF (as a control) (0.5-5 mg protein) were subjected to buffer exchange using Vivaspin 6 Concentrator units (10.000 MWCO PES, Vivascience, Cat. Nr. VS0602). Samples were concentrated to 0.5-0.7 ml and were diluted to 5 ml with 10 mM Na-phosphate buffer pH 7.2. Each sample was subjected 3 times to this concentration/buffer exchange cycle.

### 5.4 Anion Exchange Chromatography of G-CSF and HES-Modified Forms Thereof on a DEAE-Sepharose Column

G-CSF samples after HES-modification and, for comparison, samples of unmodified G-CSF were purified and analyzed by anion exchange chromatography at room temperature by using an ÄKTA explorer 10 system as described. Aliquots of G-CSF either before or after HESylation were dialyzed by ultrafiltration against said buffer A (10 mM Na-phosphate, pH 7.2) or were diluted with about 13 volumes of buffer A. The column containing 2 ml DEAE-Sepharose (DEAE-Sepharose CL-6B, Pharmacia Kat. Nr. 17-0710-01) was regenerated by applying 5.0 column volumes (CV) of 6.5 M guanidine/HCl, 5.0 CV buffer A, 5.0 CV of buffer C (1.5 M NaCl in 1.0 mM Na-phosphate, pH 7.2) and then 10 CV of buffer A. The samples (0.8-4.5 ml in 10 mM Na-phosphate buffer pH 7.2) were then injected by using a flow rate of 0.6 mL/min. Following washing of the sample loop with 10 ml (2 ml sample loop) or 20 ml (5 ml sample loop) buffer A, depending on the sample applied, the column was further washed with 0-22 CV of buffer A (flow rate 0.8 ml/min). Elution was performed by applying a linear gradient from 0-100% buffer B over 5 CV and an isocratic run with 2.5 CV of 100% buffer B using a flow rate of 0.6 ml/min. The column was re-equilibrated with 5 CV of buffer A and was regenerated as detailed above by using a flow rate of 1 ml/min.

If required, samples were concentrated using a Vivaspin concentrator and buffer exchange was performed as described above. Samples were stored at 0-8° C. in 10 mM Na-Acetat buffer pH 4.0 before or after sterile filtration using a 0.2 μm filtrations unit, Corning, Cat. No. 431215). The following samples were prepared for in-vitro bioassays and for further analytical analysis. Protein concentration was determined as described in section 6.1 below:
I. 0401-15/A33, 0.44 mg/ml, volume 500 μl G-CSF (E. coli)
II. 0402-03/A60, 0.35 mg/ml, volume=600 μl H-G-CSF (G-CSF HES modified, 10/0.4)
III. 0401-13/A32, 0.28 mg/ml, volume=900 μl G-CSF (E. coli) HES modified, 10/0.4
IV. 0401-28/A58; 0.60 mg/ml, volume=350 μl Neupogen
V. 0401-28/A57, 0.50 mg/ml, volume=400 μl Neulasta

### 5.5. Further Analysis of G-CSF Samples

Aliquots of the samples were analyzed for their protein content and for modifications.

### 5.5(a) G-CSF Protein Quantitation by RP-HPLC

G-CSF protein content of the samples was quantitated using the unmodified protein preparation (concentration: 0.453 mg/ml) as a standard.

A Dionex HPLC system consisting of a pump P 680 A HOG, degassing unit Degasys DG 1210, an autosampler and injector ASI-100, a sample loop 250 μl, a thermostatted column department TCC 100 along with a UV/Vis-Detektor UVD170U equipped with a Software Chromeleon Chromatography Management System was used. A precolumn CC 8/4 Nucleosil 120-5 C4, Macherey-Nagel, Cat. No. 721889, and a separation column 40 C-4 Nucleosil MPN, 5 μm, 125×4 mm RP-column (Macherey-Nagel, ordering No. 7200 45.40) were used. Solvent A was H₂O plus 0.06% (v/v) trifluoroacetic acid and solvent B was 90% acetonitrile in H₂O, containing 0.06% (v/v) trifluoroacetic acid; flow rate was: 1 ml/min. UV detection was at 214, 221, 260 and at 280 nm wavelength.

Samples of approximately 10-20 μg were injected into a RP-HPLC column. The following gradient was used:

| | | | |
|---|---|---|---|
| 0-5 | min: | 0-10% | B |
| 17 | min: | 10-45% | B |
| 35 | min: | 45-80% | B |
| 36 | min: | 80-100% | B |
| 38 | min: | 100% | B |
| 39 | min: | 10% | B |
| 45 | min: | 10% | B |

The resulting peak area at the elution position of the standard G-CSF preparation was used and compared to the reference standard by comparing the peak appearing at around 29 min at 280 nm wavelength.

### 5.5(b) Reduction+Carboxamidomethylation of G-CSF Protein

Aliquots from the G-CSF protein samples were reduced and carboxamidomethylated as described elsewhere (Guillermina Forno, Mariela Bollati Fogolin, Marcos Oggero, Ricardo Kratje, Marina Etcheverrigaray, Harald S. Conradt, Manfred Nimtz (2004) N- and O-linked carbohydrates and glycosylation site occupancy in recombinant human granulocyte-macrophage colony-stimulating factor secreted by a Chinese hamster ovary cell line; European J. Biochem, 273 (5), 907-919). Carboxamidomethylation leads to modified cystein residues. Endoproteinase Glu-C digestion of the carboxamidomethylated protein was performed in 25 mM NH₄HCO₃ containing 1 M urea at pH 7.8 and using an enzyme/substrate ratio of 0.2:10 for 18-24 hours.

### 5.5(c) Separation of Endo-Glu-C Peptides by RP-HPLC

The peptides generated by the Endo-Glu-C digestion were separated on a Dionex HPLC system consisting of a pump P 680 A HPG, degassing unit Degasys DG 1210, an autosampler and injector ASI-100, a sample loop 250 μl, a thermostatted column department TCC 100 along with a UV/Vis-Detektor UVD170U equipped with a Software Chromeleon Chromatography Management System was used. A precolumn CC 8/4 Nucleosil 120-5 C4, Macherey-Nagel, Cat. No. 721889, and a separation column 40 C-4 Nucleosil MPN, 5 μm, 125×4 mm RP-column (Macherey-Nagel, ordering No. 7200 45.40) were used. Solvent A was H₂O plus 0.06% (v/v) trifluoroacetic acid and solvent B was 90% acetonitrile in H₂O, containing 0.06% (v/v) trifluoroacetic acid; flow rate was: 1 ml/min. The following gradient was applied:

| | | | |
|---|---|---|---|
| 0-5 | min: | 10% | B |
| 17 | min: | 45% | B |
| 65 | min: | 100% | B |
| 67 | min: | 100% | B |
| 69 | min: | 10% | B |
| 75 | min: | 10% | B |

UV detection was at 214, 221, 260 and at 280 nm wavelength. Peptides generated by the Endo-Glu-C digestion were separated (data not shown).

### 5.5(d) Analysis of Proteolytic Peptides by Matrix-Assisted Laser Desorption/Ionization Time-of-Flight Mass Spectrometry (MALDI/TOF/TOF-MS)

Mass spectrometry was used to detect the intact N-terminus of G-CSF's in the different samples prepared. Samples (3-5 μg) resulting from Endoproteinase Glu-C digestions of reduced and carboxamidomethylated protein samples were used directly for MS-analysis (without the RP-HPLC of step 6.3) and purified using ZipTip pipette tips containing C18 reversed-phase material according to the manufacturer's instructions. After washing with 0.1% (v/v) formic acid, elution of peptides was performed with 10 μl 0.1% (v/v) formic acid in 60% (v/v) acetonitrile.

Proteolytic (Endo-Glu-C) peptide fragments were analyzed with a Bruker ULTRAFLEX time-of-flight (TOF/TOF) instrument in the linear positive ion mode using a matrix of 22.4 mg 3,5-dimethoxy-4-hydroxy-cinnamic acid in 460 μl acetonitrile and 600 μl 0.1% (v/v) trifluoroacetic acid in H₂O; (glyco)-peptides were measured using a matrix of 19 mg α-cyano-4-hydroxycinnamic acid in the same solvent mixture using the reflectron for enhanced resolution. Sample solutions of 1 μl and an approximate concentration of 1-10 pmoll·μl⁻¹ were mixed with equal amounts of the respective matrix. This mixture was spotted onto a stainless steel target and dried at room temperature before analysis. Spectra were recorded in the mass range 900-5000 dalton. The following Table correlates the expected masses with the respective G-CSF peptides.

TABLE
Theoretical (monoisotopic] masses of Endo-Glu-C peptides resulting from XM02

| Mass | Observed | aa | | |
|---|---|---|---|---|
| (Dalton) | in this study | position | artif. modification(s) | peptide sequence |
| 2132.11 | + | 1-20 | Cys_CAM: | MTPLGPASSLPQSFLLKCLE |
| | | | pos18 | |
| | | | m/z 2189.13 | |
| 1512.81 | + | 21-34 | — | QVRKIQGDGAALQE |
| 1534.74 | + | 35-47 | Cys_CAM: | KLCATYKLCHPEE |
| | | | pos 37, 43 | |
| | | | m/z 1648.78 | |
| 4942.63 | − | 48-94 | Cys_CAM: | LVLLGHSLGIPWAPLSSCPS |
| | | | Pos 65, 75 | QALQLAGCLSQLHSGLFLYQ |
| | | | m/z 5056.68 | GLLQALE |
| 502.25 | − | 95-99 | — | GISPE |
| 2835.37 | − | 100-124 | — | LGPTLDTLQLDVADFATTIW |
| | | | | QQMEE |
| 4026.08 | + | 125-163 | — | LGMAPALQPTQGAMPAFASA |
| | | | | FQRRAGGVLVASHLQSFLE |
| 1438.83 | + | 164-175 | — | VSYRVLRHLAQP |

Cystein residues were carboxamidomethylated; peptides marked as fat were detected in MALDI/TOF spectrum of the non modified G-CSF.

The N-terminal Endo-Glu-C peptide (MTPLGPASSLPQSFLLKCLE; m/z 2189.1) comprising position 1-20 of the protein was detected in MALDI/TOF-MS spectra of samples after proteolytic treatment of G-CSF with endoproteinase Glu-C as described above.

### 5.6. Results

### 5.6(a) Purification of G-CSF and HES Modified Variants

A32, A60 and non modified G-CSF were subjected to purification using a DEAE-Sepharose CL-6B column as described under A4.

In the case of the unmodified sample 0401-15/A33, no significant absorption at 280 nm was detected in the flow-through and the protein eluted at a concentration of 40-50% buffer B (0.16-0.20 M NaCl) in a volume of 6 ml, with a specific peak area of 660 mAU×ml×mg⁻¹ at 280 nm.

The sample 0401-14/A32 (derived from 0401-15/A33; HESylation with AldehydroHES 10/0.4) eluted over a large range of the gradient at a concentration of buffer B from 20-80% (0.08-0.32 M NaCl) in a volume of 12 ml. About 90% of the total peak area detected at 280 nm was found in the flow-through, containing about 50% of the total protein with an apparently slightly higher molecular mass when compared to the eluted protein, as detected by SDS-PAGE analysis as shown in FIG. 13 above.

The sample 0402-03/A60 (HESylated with HES10/0.4, following the overall procedure of Example 4.2) eluted in a volume of 10.5 ml at a similar concentration of 20-80% buffer B. In this case, about 35% of the total peak area detected at 280 nm was found in the flow-through, however, by SDS-PAGE analysis, no unbound protein was detected in this fraction. When compared to the specific peak area of sample 0401-15/A33, the protein content in the eluate of the sample 0402-03/A60 was 45% higher than the stated protein amount that was applied to the column.

Recovery of proteins was calculated based on the peak area (A280 nm) of the eluting fractions compared to the non modified G-CSF protein.

TABLE 1
Comparison of the peak areas at 280 nm detection

| | | Calculated | | Yield in eluate | |
|---|---|---|---|---|---|
| DEAE- | | protein | Eluate | compared to | |
| Sepharose | | amount for | Area [280 nm] | eluate from run | |
| Run no. | Description | injection | (mAU × ml) | DS01 A33 ** | |
| DS01 A33 | G-CSF | 0.5 mg | 330 | (0.50 | mg) |
| DS03 A32 | Hesylated | 4.0 mg | 1560 | 2.36 | mg |
| | HES 10/0.4 | | | | |
| DS04 A60 | Hesylated | 0.9 mg | 870 | 1.32 | mg |
| | HES 10/0.4 | | | | |

** RP-HPLC quantitation of the protein confirmed these results

### 5.6(b) Analysis of Proteins by Peptide Mapping and MALDI/TOF MS After Treatment with Endoproteinase Glu-C

The N-terminal peptide resulting from endoproteinase Glu-C digestion of both the carboxamidomethylated unmodified G-CSF (FIG. 14) and the market product Neupogen (data not shown), was clearly detected by MALDI/TOF-MS (MTPLGPASSLPQSFLLKC*LE, m/z 2189.1; cystein carboxamidomethylated). This signal was absent in samples subjected to HES-modification by reductive amination (FIG. 15) and in Neulasta (data not shown), indicating modification of this peptide. In the case of HES-modified G-CSF, wherein modification was carried out by activated ester chemistry, the N-terminal peptide was detected at a relative signal intensity comparable to that of the non modified starting material A32 (FIG. 16) indicating that HES modification of this derivatives has been achieved at different amino acid side chains.

N-terminal sequencing of HES modified G-CSF (sample A33 and the market product Neulasta) revealed a blocked N-terminus suggesting that in fact the N-terminal methionine residue of this protein derivative is modified by HES derivative. Since the signal corresponding to the peptide comprising amino acid residues pos. 35-47 (KLCATYKLCHPEE; both cysteine residues carboxamidomethylated m/z 1648.78) was not detected in sample A60, it is concluded that one or both lysine residues (at pos 35 and pos 41) might be modified by HES.

### REFERENCES

Guillermina Forno, Mariela Bollati Fogolin, Marcos Oggero, Ricardo Kratje, Marina Etcheverrigaray, Harald S. Conradt, Manfred Nimtz (2004) N- and O-linked carbohydrates and glycosylation site occupancy in recombinant human granulocyte-macrophage colony-stimulating factor secreted by a Chinese hamster ovary cell line Eur. J. Biochem, 271 (5), 907-919
Nimtz, M., Grabenhorst, E., Conradt H. S., Sanz, L. & Calvete, J. J. (1999) Structural characterization of the oligosaccharide chains of native and crystallized boar seminal plasma spermadhesin PSP-I and PSP-II glycoforms. Eur. J. Biochem. 265, 703-718.
Nimtz, M., Martin, W., Wray, V., Klöppel, K.-D., Agustin, J. & Conradt, H. S. (1993) Structures of sialylated oligosaccharides of human erythropoietin expressed in recombinant BHK-21 cells. Eur J. Biochem. 213, 39-56
Nimtz, M., Noll G., Pâques, E. & Conradt, H. S. (1990) Carbohydrate structures of human tissue plasminogen activator variant expressed in recombinant Chinese hamster ovary cells. FEBS Lett. 271, 14-18.
Schröter, S., Derr, P., Conradt, H. S., Nimtz, M., Hale, G. & Kirchhoff, C. (1999) Male-specific modification of human CD52. J. Biol. Chem. 274, 29862-29873
E. Grabenhorst and H. S. Conradt (1999) The Cytoplasmic, Transmembrane and the Stem Regions of Glycosyltransferases specify their in vivo functional sublocalization and stability in the Golgi
   J. Biol. Chem., 274, 36107-36116
E. Grabenhorst, A. Hoffmann, M. Nimtz, G. Zettlmeiβl and H. S. Conradt (1995) Construction of stable BHK-21 cells coexpressing human secretory glycoproteins and human Galβ1-4GlcNAc-R □2,6-sialyltransferase: □2,6-linked NeuAc is preferably attached to the Galβ1-4GlcNAcβ1-2Manβ1-3-branch of biantennary oligosaccharides from secreted recombinant-trace protein.
   Eur. J. Biochem., 232, 718-725

### Example 6

### In Vitro Results of the G-CSF-Conjugate Obtained in Examples 2.5 and 4.2 and Purified According to Example 5: Mitogenicity of G-CSF Variants for Mouse NFS-60 Cells

G-CSF is known for its specific effects on the proliferation, differentiation, an activation of hematopoietic cells of the neutrophilic granulocyte lineage. The mitogenic capacity of G-CSF variants was tested using mouse NFS-60 cells (N. Shirafuji et al., Exp. Hematol. 1989, 17, 116-119). Cells grown in RPMI medium with 10% fetal calf serum (Gibco INVITROGEN GmbH, Karlsruhe, D) containing 5-10% WEHI-3B (DSMZ, Braunschweig, D; cultivated as described by the DSMZ) conditioned medium as source of exogenous IL-3 were harvested by centrifugation, washed and aliquoted at 100,000 cells per well in a 24-well plate. Cells were allowed to adapt for 1 hour at 37° C. in RPMI medium without WEHI-3B conditioned media before G-CSF growth factors sample diluted in the same media were added. NFS-60 cells were exposed to purified G-CSF variants for 3 days at 37° C. and than the cells were electronically counted (Casy TT Cell Counter, Schärfe System, Reutlingen, D). The results are summarised in FIG. 12. As seen in FIG. 12, the different G-CSF variants (0.5-50 pg/ml) were able to stimulate an increase in the number of cells after 3 days compared to medium that did not contain added growth factors.

Unmodified control proteins G-CSF/A33 and G-CSF/A58 stimulated cells at a very similar extend (ED50=5-10 pg/ml) while G-CSF conjugates G-CSF/A60 G-CSF/A32 and G-CSF/A57 showed only a minor decrease in activity if compared to the unmodified version (ED50=10-25 pg/ml).

### (see FIG. 8)

### Example 7

### Synthesis of G-CSF-Conjugates

### Example 7.1

### Synthesis of the Aldehydro-HES Derivatives

### Example 7.1(a)

### Synthesis of AminoHES10/0.4

5.12 g of oxo HES10/0.4 (MW=10000 D, DS=0.4, Supramol Parenteral Colloids GmbH, Rosbach-Rodheim, D according to DE 196 28 705 A1) were heated over night at 80° C. in vacuo and dissolved under nitrogen in 25 mL dry dimethyl sulphoxide (Fluka, Sigma-Aldrich Chemie GmbH, Taufkirchen, D) and 5.13 mL of 1,4-diaminobutane were added. After stirring at 40° C. for 17 h the reaction mixture was added to 150 mL of an ice-cold 1:1 mixture of acetone and ethanol (v/v). The precipitated product was collected by centrifugation at 4° C., washed with 40 mL of an ice-cold 1:1 mixture of acetone and ethanol (v/v) and collected by centrifugation. The crude product was dissolved in 80 mL water, dialysed for 4 d against water (SnakeSkin dialysis tubing, 3.5 kD cut off, Perbio Sciences Deutschland GmbH, Bonn, D) and lyophilized. The yield of isolated product was 67%.

### Example 7.1(b)

### Synthesis of AldehydroHES10/0.4

105 mg 4-formylbenzoic acid and 135 mg 1-hydroxy-1H-benzotriazole (both Aldrich, Sigma-Aldrich Chemie. GmbH, Taufkirchen, D) were dissolved in 7 mL N,N-dimethylformamide (Peptide synthesis grade, Biosolve, Valkenswaard, NL) and 135 μL N,N′-diisopropylcarbodiimide (Fluka, Sigma-Aldrich Chemie GmbH, Taufkirchen, D) were added. After incubation at 21° C. for 30 min, 0.7 g of aminoHES10/0.4 (synthesised as described in 1.1) were added. After shaking for 18 h at 22° C., the reaction mixture was added to 42 mL of an ice-cold 1:1 mixture of acetone and ethanol (v/v). The precipitated product was collected by centrifugation at 4° C., re-dissolved in 5 mL DMF and precipitated with 42 mL ethanol/acetone as described above. After centrifugation, the collected precipitate was dissolved with water, dialysed for 1 d against water (SnakeSkin dialysis tubing, 3.5 kD cut off, Perbio Sciences Deutschland GmbH, Bonn, D) and lyophilized. The yield of isolated product was 95%.

### Example 7.1(c)

### Synthesis of AminoHES10/0.7

6.02 g of oxo-HES10/0.7 (MW=10000 D, DS=0.7, Supramol Parenteral Colloids GmbH, Rosbach-Rodheim, D, according to DE 196 28 705) were dissolved under nitrogen in 32 mL dry dimethyl sulphoxide (Fluka, Sigma-Aldrich Chemie GmbH, Taufkirchen, D) and 6.03 mL of 1,4-diaminobutane were added. After stirring at 40° C. for 17 h the reaction mixture was added to 150 mL of an ice-cold 1:1 mixture of acetone and ethanol (v/v). The precipitated product was collected by centrifugation at 4° C., washed with 40 mL of an ice-cold 1:1 mixture of acetone and ethanol (v/v) and collected by centrifugation. The crude product was dissolved in 80 mL water, dialysed for 4 d against water (SnakeSkin dialysis tubing, 3.5 kD cut off, Perbio Sciences Deutschland GmbH, Bonn, D) and lyophilized. The yield of isolated product was 52%.

### Example 7.1(d)

### Synthesis of AldehydroHES10/0.7

150 mg 4-formylbenzoic acid and 230 mg 1-hydroxy-1H-benzotriazole (both Aldrich, Sigma-Aldrich Chemie GmbH, Taufkirchen, D) were dissolved in 10 mL N,N-dimethylformamide (Peptide synthesis grade, Biosolve, Valkenswaard, NL) and 204 μL N,N′-diisopropylcarbodiimide (Fluka, Sigma-Aldrich Chemie GmbH, Taufkirchen, D) were added. After incubation at 21° C. for 30 min, 1 g of aminoHES10/0.7 (synthesised as described in 1.3) were added. After shaking for 19 h at 22° C., the reaction mixture was added to 84 mL of ice-cold 2-propanol. The precipitated product was collected by centrifugation at 4° C., re-dissolved in 50 mL water, dialysed for 2 d against water (SnakeSkin dialysis tubing, 3.5 kD cut off, Perbio Sciences Deutschland GmbH, Bonn, D) and lyophilized. The yield of isolated product was 83%.

### Example 7.1(e)

### Synthesis of AminoHES30/0.4

5 g of oxo-HES 30/0.4 (MW=30000 D, DS=0.4, Supramol Parenteral Colloids GmbH, Rosbach-Rodheim, D, using molar ratios of the ingredients according to DE 196 28 705 A1) were heated over night at 80° C. in vacuo and were then dissolved under nitrogen in 28 mL dry dimethyl sulphoxide (Fluka, Sigma-Aldrich Chemie GmbH, Taufkirchen, D) and 1.67 mL of 1,4-diaminobutane were added. After stirring at 40° C. for 17 h the reaction mixture was added to 175 mL of an ice-cold 1:1 mixture of acetone and ethanol (v/v). The precipitated product was collected by centrifugation at 4° C. The crude product was dissolved in 40 mL water, dialysed for 2 d against water (SnakeSkin dialysis tubing, 3.5 kD cut off, Perbio Sciences Deutschland GmbH, Bonn, D) and lyophilized. The yield of isolated product was not determined.

### Example 7.1(f)

### Synthesis of AldehydroHES30/0.4

130 mg 4-formylbenzoic acid and 153 mg 1-hydroxy-1H-benzotriazole (both Aldrich, Sigma-Aldrich Chemie GmbH, Taufkirchen, D) were dissolved in 36 mL N,N-dimethylformamide (Peptide synthesis grade, Biosolve, Valkenswaard, NL) and 110 μL N,N′-diisopropylcarbodiimide (Fluka, Sigma-Aldrich Chemie GmbH, Taufkirchen, D) were added. After incubation at 21° C. for 30 min, 2.61 g of aminoHES30/0.4 (synthesised as described in 1.5) were added. After shaking for 22.5 h at 22° C., the reaction mixture was added to 160 mL of an ice-cold 1:1 mixture of acetone and ethanol (v/v). The precipitated product was collected by centrifugation at 4° C. and washed with an ice-cold 1:1 mixture of acetone and ethanol (v/v). After centrifugation, the precipitate was dissolved in 30 mL water, dialysed for 1 d against water (SnakeSkin dialysis tubing, 3.5 kD cut off, Perbio Sciences Deutschland GmbH, Bonn, D) and lyophilized. The yield of isolated product was 81%.

### Example 7.1(g)

### Synthesis of AminoHES30/0.7

5 g of oxo-HES 3010.7 (MW=30000 D, DS=0.7, Supramol Parenteral Colloids GmbH, Rosbach-Rodheim, D, using molar ratios of the ingredient according to DE 196 28 705 A1) were heated over night at 80° C. in vacuo and were then dissolved under nitrogen in 28 mL dry dimethyl sulphoxide (Fluka, Sigma-Aldrich Chemie GmbH, Taufkirchen, D) and 1.67 mL of 1,4-diaminobutane were added. After stirring at 40° C. for 17 h the reaction mixture was added to 175 mL of an ice-cold 1:1 mixture of acetone and ethanol (v/v). The precipitated product was collected by centrifugation at 4° C. The crude product was dissolved in 40 mL water, dialysed for 2 d against water (SnakeSkin dialysis tubing, 3.5 kD cut off, Perbio Sciences Deutschland GmbH, Bonn, D) and lyophilized. The yield of isolated product was not determined.

### Example 7.1(h)

### Synthesis of AldehydroHES30/0.7

122 mg 4-formylbenzoic acid and 144 mg 1-hydroxy-1H-benzotriazole (both Aldrich, Sigma-Aldrich Chemie GmbH, Taufkirchen, D) were dissolved in 34 mL N,N-dimethylformamide (Peptide synthesis grade, Biosolve, Valkenswaard, NL) and 103 μL N,N′-diisopropylcarbodiimide (Fluka, Sigma-Aldrich Chemie GmbH, Taufkirchen, D) were added. After incubation at 21° C. for 30 min, 2.46 g of aminoHES30/0.7 (synthesised as described in 1.7) were added. After shaking for 22.5 h at 22° C., the reaction mixture was added to 160 mL of an ice-cold 1:1 mixture of acetone and ethanol (v/v). The precipitated product was collected by centrifugation at 4° C. and washed with an ice-cold 1:1 mixture of acetone and ethanol (v/v). After centrifugation, the precipitate was dissolved in 30 mL water, dialysed for 1 d against water (SnakeSkin dialysis tubing, 3.5 kD cut off, Perbio Sciences Deutschland GmbH, Bonn, D) and lyophilized. The yield of isolated product was 87%.

### Example 7.1(i)

### Synthesis of AminoHES50/0.7

6.09 g of oxo-HES 50/0.7 (MW=50000 D, DS=0.7, Supramol Parenteral Colloids GmbH, Rosbach-Rodheim, D, using molar rations of the ingredients according to DE 196 28 705 A1) were heated over night at 80° C. in vacuo and were then dissolved under nitrogen in 32 mL dry dimethyl sulphoxide (Fluka, Sigma-Aldrich Chemie GmbH, Taufkirchen, D) and 1.22 mL of 1,4-diaminobutane were added. After stirring at 40° C. for 17 h the reaction mixture was added to 150 mL of an ice-cold 1:1 mixture of acetone and ethanol (v/v). The precipitated product was collected by centrifugation at 4° C., washed with 40 mL of an ice-cold 1:1 mixture of acetone and ethanol (v/v) and collected by centrifugation. The crude product was dissolved in 80 mL water, dialysed for 4 d against water (SnakeSkin dialysis tubing, 3.5 kD cut off, Perbio Sciences Deutschland GmbH, Bonn, D) and lyophilized. The yield of isolated product was 82%.

### Example 7.1(j)

### Synthesis of AldehydroHES50/0.7

125 mg 4-formylbenzoic acid and 174 mg 1-hydroxy-1H-benzotriazole (both Aldrich, Sigma-Aldrich Chemie GmbH, Taufkirchen, D) were dissolved in 38 mL N,N-dimethylformamide (Peptide synthesis grade, Biosolve, Valkenswaard, NL) and 155 μL N,N′-diisopropylcarbodiimide (Fluka, Sigma-Aldrich Chemie GmbH, Taufkirchen, D) were added. After incubation at 21° C. for 30 min, 3.8 g of aminoHES50/0.7 (synthesised as described in 1.9) were added. After shaking for 19 h at 22° C., the reaction mixture was added to 160 mL of an ice-cold 1:1 mixture of acetone and ethanol (v/v). The precipitated product was collected by centrifugation at 4° C., re-dissolved in 20 mL N,N-dimethylformamide and precipitated with 80 mL of an ice-cold 1:1 mixture of acetone and ethanol (v/v) as described above. After centrifugation, the precipitate was dissolved in 50 mL water, dialysed for 2 d against water (SnakeSkin dialysis tubing, 3.5 kD cut off, Perbio Sciences Deutschland GmbH, Bonn, D) and lyophilized. The yield of isolated product was 77%.

### Example 7.2

### Synthesis of the HES-G-CSF Conjugates by Reductive Amination

### Example 7.2(a)

### Buffer Exchange A

33 mL of a 0.454 mg/mL solution of hG-CSF (XM02, BioGeneriX AG, Mannheim, D) in 10 mM sodium acetate, 50 mg/mL sorbitol and 0.004% Tween 80 at pH 4.0 were concentrated by diafiltration at 0° C. to 4 mL with a Vivaspin 15R concentrator (VS15RH11, 5 KD MWCO, Vivascience AG, Hannover, D) and re-diluted to 15 mL with a 0.1 M sodium acetate buffer at pH 5.0. This diafiltration was repeated twice. The final concentration in the last diafiltration step was 3 mg/mL.

### Example 7.2(b)

### Reaction of hG-CSF with AldehydroHES Derivatives of Examples 7.1(b), 7.1(d) and 7.1(j)

To 1.67 mL of a solution of hG-CSF after buffer exchange into 0.1 M sodium acetate buffer, pH 5.0 (as described in 7.2(a) above) 1.67 mL of a solution of the HES-derivative and 1.67 mL of a 60 mM solution of sodium cyanoborohydride, both in the same buffer, were added and the solution was incubated for 15.5 h at 4° C. All solutions were cooled to 0° C. before mixing.

The following final HES concentrations were employed:

39.4 mg/mL for the HES derivatives prepared according to example 7.1(b) and 7.1(d).
197 mg/mL for the HES derivative prepared according to example 7.1(j).
197 mg/mL HES50/0.7 (MW=50000 D, DS=0.7, Supramol Parenteral Colloids GmbH, Rosbach-Rodheim, D) as reaction control.

The reaction mixtures were analysed by gel electrophoresis (see FIG. 17)

### Example 7.2(c)

### Buffer Exchange B

20 mL of a 0.454 mg/mL solution of hG-CSF (XM02, BioGeneriX AG, Mannheim, D) in 10 mM sodium acetate, 50 mg/mL sorbitol and 0.004% Tween 80 at pH 4.0 was concentrated by diafiltration at 15° C. to 4 mL with a Vivaspin 15R concentrator (VS15RH11, 5 KD MWCO, Vivascience AG, Hannover, D) and re-diluted to 15 mL with a 0.1 M sodium acetate buffer at pH 5.0. This diafiltration was repeated twice. The final concentration in the last diafiltration step was 1.5 mg/mL.

### Example 7.2(d)

### Reaction of hG-CSF with AldehydroHES Derivatives of Examples 7.1(f) and 7.1(h)

To 3.3 mL of a solution of hG-CSF after buffer exchange into 0.1 M sodium acetate buffer, pH 5.0 (as described in example 7.2(c) above) 3.3 mL of a solution of 789 mg of the HES-derivative and 3.3 mL of a 60 mM solution of sodium cyanoborohydride, both in the same buffer, were added and the solution was incubated for 30 h at 4° C. All solutions were cooled to 0° C. before mixing.

After 17 h samples were removed for reaction control. The reaction mixtures were analysed by gel electrophoresis (see FIG. 18).

### Example 7.3

### Synthesis of HES-GCFS conjugates by N,N′-succinimidyl carbonate coupling

### Example 7.3(a)

### Synthesis of G-CSF Conjugates by Reaction of Hydroxyethyl Starch Having a Reactive Ester Group with G-CSF

400 mg of oxo-HES10/0.7 (prepared by Supramol Parenteral Colloids GmbH, Rosbach-Rodheim, D; according to DE 196 28 705 A1, degree of oxidation of oxo-HES was 95%) were dissolved in 1 ml anhydrous DMF. To this solution, 21 mg of N,N′-disuccinimidyl carbonate were added, and the mixture was stirred for 2 h at room temperature. The resulting solution had a reactive HES concentration of 40 percent by weight.

A solution of G-CSF (Strathmann Biotec AG, Hamburg, D), having a concentration of about 0.5 mg G-CSF/ml, was concentrated to a concentration of 10 mg/ml by ultracentrifugation at a cut-off of 100 kD using a cooling centrifuge.

To 0.5 ml of this concentrated G-CSF solution, 180 μl of a sodium bicarbonate solution were added. Subsequently, 3 portions (100 μl each) of the reactive HES solution were added dropwise to the protein solution, until; after about 30 min., the reaction had come to an end. Thus, the overall molar ratio of reactive HES:G-CSF was about 50:1. Then, the pH of the mixture was adjusted to 4.0 using 0.1 N HCl.

A HPGPC analysis (High-Performance Gel Permeation Chromatography) gave a yield of more than 95%. No non-reacted G-CSF could be detected. This result is shown in FIG. 19.

### Example 7.4

### In Vitro Assay

### Mitogenicity of G-CSF Variants for Mouse NFS-60 Cells

G-CSF is known for its specific effects on the proliferation, differentiation and activation of hematopoietic cells of, the neutrophilic granulocyte lineage. The mitogenic capacity of G-CSF variants was tested using mouse NFS-60 cells (N. Shirafuji et al., Exp. Hematol. 1989, 17, 116-119). Cells grown in RPMI medium with 10% fetal calf serum (Gibco INVITROGEN GmbH, Karlsruhe, D) containing 5-10% WEHI-3B (DSMZ, Braunschweig, D; cultivated as described by the DSMZ) conditioned medium as source of exogenous IL-3 were harvested by centrifugation, washed and aliquoted at 100,000 cells per well in a 24-well plate. Cells were allowed to adapt for 1 hour at 37° C. in RPMI medium without WEHI-3B conditioned media before G-CSF growth factor samples diluted in the same media were added. NFS-60 cells were exposed to purified G-CSF variants (purification according to examples 5.3, 5.4, protein quantification according to example 5.5(a)):

Neupogen®, Neulasta® both from Amgen,
“HES-GCFS10/0.4 conjugate” prepared in example 7.2(b),
“HES-GCFS10/0.7 conjugate” prepared in example 7.2(b),
“HES-GCFS30/0.4 conjugate” prepared in example 7.2(d),
“HES-GCFS30/0.7 conjugate” prepared in example 7.2(d),
“HES-GCFS50/0.7 conjugate” prepared in example 7.2(b),
“HES-GCFS10/0.7 conjugate (Supramol)” prepared according to example 7.3(a),
“Mock incubation” (=reaction control, 197 mg/ml HES50/0.7, MW 50000D, DS 7, Supramol Parenteral Colloids GmbH, Rosbach Rodheim, Germany),
for 3 days at 37° C. and then the cells were electronically counted (Casy TT Cell Counter, Schärfe System, Reutlingen, D). The results are summarised in Table 2 and FIG. 20. In all cases, the amounts of protein given in Table 2 and FIG. 20 represent the G-CSF content of the conjugates only and are based on the concentrations determined by GlycoThera. As can be seen in FIG. 20, all of the different G-CSF variants (2.5-250 pg/ml) were able to stimulate an increase in the number of cells after 3 days compared to a medium that did not contain added growth factors. All variants reached the same maximum stimulation level at a concentration of 250 pg/ml.

TABLE 2
Proliferation of mouse NFS-60 cells, induced by G-CSF variants

| | Concentration [pg/ml] | | | | | | | | | | | | |
|---|---|---|---|---|---|---|---|---|---|---|---|---|---|
| | 0 | 2.5 | 2.8 | 5 | 5.7 | 10 | 11.3 | 25 | 28.4 | 50 | 56.7 | 250 | 283.5 |
| Neupogen | 0.44 | 0.86 | | 1.20 | | 1.69 | | 2.33 | | 2.49 | | 2.41 | |
| HES-GCSF | 0.44 | 0.72 | | 0.93 | | 1.44 | | 2.14 | | 2.41 | | 2.41 | |
| 10/0.7 | | | | | | | | | | | | | |
| HES-GCSF | 0.44 | 0.72 | | 0.97 | | 1.40 | | 2.17 | | 2.67 | | 2.75 | |
| 10/0.4 | | | | | | | | | | | | | |
| HES-GCSF | 0.44 | 0.62 | | 0.70 | | 0.97 | | 1.68 | | 2.15 | | 2.32 | |
| 50/0.7 | | | | | | | | | | | | | |
| Mock- | 0.44 | 0.85 | | 1.31 | | 1.91 | | 2.38 | | 2.47 | | 2.41 | |
| incubation | | | | | | | | | | | | | |
| HES-GCSF | 0.44 | 0.82 | | 1.21 | | 1.62 | | 2.28 | | 2.50 | | 2.60 | |
| 30/0.4 | | | | | | | | | | | | | |
| HES-GCSF | 0.44 | 0.80 | | 1.09 | | 1.66 | | 2.20 | | 2.35 | | 2.44 | |
| 30/0.7 | | | | | | | | | | | | | |
| Neulasta | 0.44 | 0.63 | | 0.80 | | 1.12 | | 1.83 | | 2.25 | | 2.33 | |
| HES-GCSF | 0.44 | | 0.73 | | 1.13 | | 1.58 | | 2.24 | | 2.48 | | 2.46 |
| 10/0.7 | | | | | | | | | | | | | |
| (Supramol) | | | | | | | | | | | | | |

### Example 7.5

### In Vivo Biological Effects of hG-CSF Conjugates in Rats

Upon arrival, the rats [male CRL:CD® rats (7 weeks old), Charles River Deutschland GmbH, Sanghofer Weg 7, D-97633 Sulzfeld)] were randomly sorted into groups of 5. After 7 days acclimatization, rats in poor condition were excluded and replaced by spare animals. The weight of the rats upon arrival was 181-203 g.

Each group of five randomly selected rats was then intravenously administered 100 μg protein per kg body weight (injection speed 15 sec/dosis, vehicle: 5 ml PBS/kg bodyweight) of the following non-conjugated or conjugated G-CSF samples (purified according to examples 5.3, 5.4, protein quantification according to example 5.5(a)): Neupogen® and Neulasta®, both from Amgen,

“HES-GCSF10/0.4 conjugate” (10/0.4) prepared in example 7.2(b),
“HES-GCSF10/0.7 conjugate” (10/0.7) prepared in example 7.2(b),
“HES-GCSF30/0.4 conjugate” (30/6.4) prepared in example 7.2(d),
“HES-GCSF30/0.7 conjugate” (30/0.7) prepared in example 7.2(d),
“HES-GCSF50/0.7 conjugate” (50/0.7) prepared in example 7.2(b),
“HES-GCSF10/0.7 Supramol” (S10/0.7) prepared according to example 7.3(a)
“Mock incubation” (=reaction control, 197 mg/ml HES50/0.7, MW 50000D, DS 7, Supramol Parenteral Colloids GmbH, Rosbach Rodheim, Germany) and a vehicle control.

Blood samples from all animals of approx. 200 μl EDTA whole blood were taken from the retrobulbar venous plexus under light ether anaesthesia. On test day −5 blood was taken once in the morning from all animals after overnight fasting. On test days 1 to 8 blood was taken twice daily at an interval of 12 hours. The first blood sample on day 1 was taken prior to G-CSF/GCSF-conjugate administration.

White blood cell (WBC) counts were carried out using a Bayer ADVIA™ 120 (Fernwald, Germany). The results are shown in FIG. 21.

1. A method for preparing a conjugate comprising a protein and a polymer or a derivative thereof, wherein the polymer is a hydroxyalkyl starch (HAS) and the protein is a granulocyte colony stimulating factor (G-CSF), the method comprising reacting at least one functional group A of the polymer or the derivative thereof with at least one functional group Z of the protein and thereby forming a covalent linkage, wherein Z is selected from the group consisting of an amino group, a thiol group, an aldehyde group and a keto group, and
wherein, in case Z is an aldehyde group or a keto group, A comprises an amino group forming said linkage with Z, or
wherein, in case Z is an amino group, A is selected from the group consisting of a reactive carboxy group and an aldehyde group, a keto group or a hemiacetal group,
wherein, in case A is an aldehyde group, a keto group or a hemiacetal group, the method further comprises introducing A in the polymer to give a polymer derivative
by reacting the polymer with an at least bifunctional compound, one functional group of which reacts with the polymer and at least one other functional group of which is an aldehyde group, a keto group or a hemiacetal group, or is a functional group which is further chemically modified to give an aldehyde group, a keto group or a hemiacetal group, or
by oxidizing the polymer to give at least one aldehyde group, or
wherein, in case A is a reactive carboxy group, the method further comprises introducing A in the polymer to give a polymer derivative
by selectively oxidizing the polymer at its reducing end and activating the resulting carboxy group, or
by reacting the polymer at its non-oxidized reducing end with a carbonic diester, or
wherein, in case Z is a thiol group, A comprises
a maleimido group or
a halogenacetyl group
forming said linkage with Z.

2. The method as claimed in claim 1 wherein the hydroxyalkyl starch is hydroxyethyl starch.

3. The method as claimed in claim 2 wherein the hydroxyethyl starch has a molecular weight of from 2 to 200 kD.

4. The method as claimed in claim 1, wherein Z is an aldehyde group or a keto group.

5. The method as claimed in claim 4, wherein the aldehyde group or the keto group is located in a carbohydrate side chain of the protein and/or at the N-terminal group of the protein.

6. The method as claimed in claim 5, further comprising oxidizing the carbohydrate side chain of the protein and/or oxidizing the N-terminal group of the protein to give the aldehyde group or keto group.

7. The method as claimed in claim 6, wherein the oxidation reaction is carried out enzymatically or using a periodate, in each case, if necessary, after having removed a terminal sialic acid.

8. The method as claimed in claim 4, further comprising reacting the polymer at its non-oxidized reducing end with an at least bifunctional linking compound comprising a functional group capable of reacting with the non-oxidized reducing end of the polymer and a group A, prior to the reaction of the polymer derivative comprising A and the protein comprising Z.

9. The method as claimed in claim 4, wherein A is an aminooxy group or a hydrazido group.

10. The method as claimed in claim 8, wherein the at least bifunctional linking compound is a homobifunctional compound.

11. The method as claimed in claim 10, wherein the homobifunctional compound comprises two aminooxy groups.

12. The method as claimed in claim 11, wherein the homobifunctional compound is O-[2-(2-aminooxy-ethoxy)-ethyl]hydroxyl amine.

13. The method as claimed in claim 8, wherein the reaction of the polymer with the at least bifunctional linking compound is carried out in an aqueous medium.

14. The method as claimed in claim 11, wherein the reaction of the polymer with the at least bifunctional linking compound leads to an oxime linkage and/or an oxyamino linkage.

15. The method as claimed in claim 1, wherein Z is an amino group.

16. The method as claimed in claim 15, further comprising selectively oxidising the polymer at its reducing end and reacting the oxidised polymer with N,N′-disuccinimidyl carbonate at its oxidised reducing end to give a polymer derivative comprising the reactive carboxy group A.

17. The method as claimed in claim 15, further comprising reacting at least one hydroxy group of the polymer whose reducing end is not oxidised, with a carbonic diester to give the reactive carboxy group A.

18. The method as claimed in claim 17, wherein the carbonic diester is a symmetrical diester.

19. The method as claimed in claim 17, wherein the alcohol component of the ester is selected from the group consisting of N-hydroxy succinimide, sulfonate N-hydroxy succinimide, N-hydroxy benzotriazole, and nitro- or halogen-substituted phenols.

20. The method as claimed in claim 19, wherein the halogen-substituted phenol is selected from the group consisting of nitrophenol, dinitrophenol, trichlorophenol, trifluorophenol, pentachlorophenol, and pentafluorophenol.

21. The method as claimed in claim 17, wherein the reaction of the at least one hydroxy group of the polymer whose reducing end is not oxidised, with the carbonic diester to give a reactive ester group A is carried out in an anhydrous aprotic polar solvent.

22. The method as claimed in claim 21, wherein the solvent is dimethyl acetamide, dimethyl formamide or a mixture thereof.

23. The method as claimed in claim 15, wherein A is an aldehyde group, a keto group or a hemiacetal group, the method further comprising reacting the polymer with a functional group M of an at least bifunctional compound to give a polymer derivative, the at least bifunctional compound further comprising at least one other functional group Q which is the aldehyde group, keto group or hemiacetal group A.

24. The method as claimed in claim 23, wherein M comprises an amino group.

25. The method as claimed in claim 23, wherein A is an aldehyde group, keto group or hemiacetal group, the method further comprising reacting the polymer with a functional group M of an at least bifunctional compound to give a polymer derivative, the at least bifunctional compound further comprising at least one other functional group Q which is not an aldehyde group, keto group or hemiacetal group, the method further comprising reacting the functional group Q with at least one suitable compound to give the polymer derivative comprising the aldehyde group, keto group or hemiacetal group A.

26. The method as claimed in claim 24, wherein M and Q comprise an amino group.

27. The method as claimed in claim 25, wherein the at least one suitable compound comprises a carboxy group and an aldehyde group, keto group or hemiacetal group.

28. The method as claimed in claim 27, wherein the at least one suitable compound is formylbenzoic acid or 4-(4-formyl-3,5-dimethoxyphenoxy)butyric acid.

29. The method as claimed in claim 25, wherein M comprises an amino group and Q comprises a beta hydroxy amino group.

30. The method as claimed in claim 29, wherein the polymer is reacted at its oxidized reducing end with a functional group M of an at least bifunctional compound.

31. The method as claimed in claim 29, further comprising oxidizing the betahydroxyamino group to give the aldehyde group.

32. The method as claimed in claim 31, wherein the oxidation reaction is carried out using a periodate.

33. The method as claimed in claim 15, wherein the polymer is subjected to a ring-opening oxidation reaction using a periodate to give a polymer derivative having at least one aldehyde group A.

34. The method as claimed in claim 23, wherein the reaction of the polymer or the polymer derivative with the protein is a reductive amination.

35. The method as claimed in claim 34, wherein the reductive amination is carried out in the presence of NaCNBH₃.

36. The method as claimed in claim 34, wherein the reductive amination is carried out at a pH of 7 or less.

37. The method as claimed in claim 36, wherein the pH is 6 or less.

38. The method as claimed in claim 34, wherein the reductive amination is carried out at a temperature of from 0 to 25° C.

39. The method as claimed in claim 34, wherein the reductive amination is carried out in an aqueous medium.

40. The method as claimed in claim 1, wherein Z is a thiol group.

41. The method as claimed in claim 40, wherein A comprises a halogenacetyl group, the method further comprising reacting the polymer at its optionally oxidized reducing end with an at least bifunctional compound having at least two functional groups each comprising an amino group to give a polymer derivative having at least one functional group comprising an amino group, the method further comprising reacting the polymer derivative with a monohalogen-substituted acetic acid and/or a reactive monohalogen-substituted acetic acid derivative.

42. The method as claimed in claim 41, wherein the halogen is Br or I.

43. The method as claimed in claim 41, wherein the at least bifunctional compound is a diaminoalkane having from 2 to 10 carbon atoms.

44. The method as claimed in claim 41, wherein the at least bifunctional compound is adiaminopolyethylene glycol having from 1 to 5 alkylene units.

45. The method as claimed in any of claims 41, wherein the polymer is reacted with the at least bifunctional compound at its oxidized reducing end.

46. The method as claimed in claim 41, wherein the polymer derivative comprising the halogenacetyl group is reacted with the protein in the presence of a solvent comprising a mixture of dimethyl formamide and water.

47. The method as claimed in claim 40, wherein A comprises a maleimido group, the method further comprising reacting the polymer at its optionally oxidized reducing end with an at least bifunctional compound comprising a functional group U capable of reacting with the optionally oxidised reducing end, the at least bifunctional compound further comprising a functional group W capable of being chemically modified to give a maleimido group, the method further comprising chemically modifying the functional group W to give a maleimido group.

48. The method as claimed in claim 47, wherein U comprises an amino group.

49. The method as claimed in claim 47, wherein W comprises an amino group.

50. The method as claimed in claim 47, wherein the polymer derivative comprising W is reacted with an at least bifunctional compound comprising a functional group capable of being reacted with W and further comprising a maleimido group.

51. The method as claimed in claim 50, wherein the at least bifunctional compound is N-(alpha-maleimidoacetoxy)succinimide ester.

52. A conjugate obtainable by the method as claimed in claim 1.

53. The method as claimed in claim 1, wherein A is a reactive carboxy group, and wherein A was introduced in the polymer whose reducing end was not oxidized, by reacting at least one hydroxy group of the polymer with a carbonic diester, and wherein said conjugate comprises one polymer molecule and at least one protein molecule linked to the polymer via amide linkages.

54. A conjugate comprising a protein and a polymer or a derivative thereof, wherein the polymer is a hydroxyalkyl starch (HAS) and the protein is a granulocyte colony stimulating factor (G-CSF), having a structure according to the formula 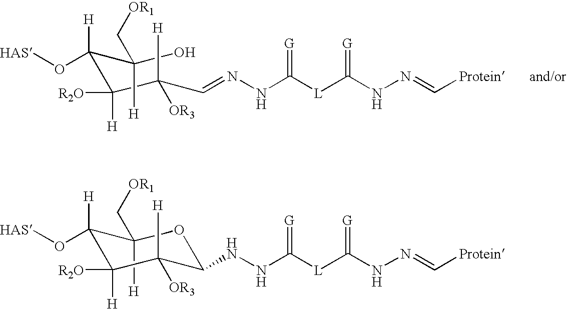
wherein R₁, R₂ and R₃ are independently hydrogen or a hydroxyalkyl group, a hydroxyaryl group, a hydroxyaralkyl group or a hydroxyalkaryl group having of from 2 to 10 carbon atoms, wherein G is selected from the group consisting of O and S, and wherein L is an optionally suitably substituted, linear, branched and/or cyclic hydrocarbon residue, optionally comprising at least one heteroatom having from 2 to 60 carbon atoms.

55. The conjugate as claimed in claim 54, wherein -L- is —(CH₂)ₙ— with n=2, 3, 4, 5, 6, 7, 8, 9, or 10.

56. A conjugate comprising a protein and a polymer or a derivative thereof, wherein the polymer is a hydroxyalkyl starch (HAS) and the protein is a granulocyte colony stimulating factor (G-CSF), having a structure according to the formula 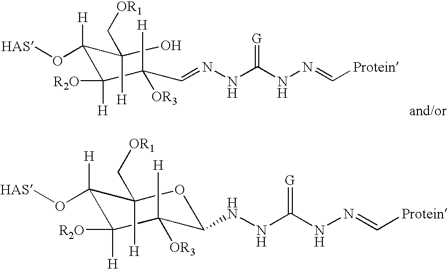
wherein R₁, R₂ and R₃ are independently hydrogen or a hydroxyalkyl group, a hydroxyaryl group, a hydroxyaralkyl group or a hydroxyalkaryl group having of from 2 to 10 carbon atoms, and wherein G is selected from the group consisting of O and S.

57. A conjugate comprising a protein and a polymer or a derivative thereof, wherein the polymer is a hydroxyalkyl starch (HAS) and the protein is a granulocyte colony stimulating factor (G-CSF), having a structure according to the formula 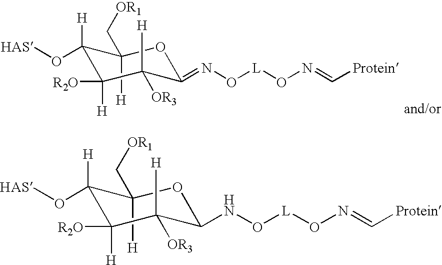
wherein R₁, R₂ and R₃ are independently hydrogen or a hydroxyalkyl group, a hydroxyaryl group, a hydroxyaralkyl group or a hydroxyalkaryl group having of from 1 to 10 carbon atoms, and wherein L is an optionally suitably substituted, linear, branched and/or cyclic hydrocarbon residue, optionally comprising at least one heteroatom having from 2 to 60 carbon atoms.

58. The conjugate as claimed in claim 57, wherein -L- is
—[(CRₐR_{b})ₘG]ₙ[CR_{c}R_{d}]ₒ—
wherein Rₐ, R_{b}, R_{c}, and R_{d} are independently hydrogen, alkyl, or aryl, wherein G is selected from the group consisting of O and S, and wherein
m 1, 2, 3 or 4, wherein the residues Rₐ and R_{b} may be the same or different in the m groups (CRₐR_{b});
n 0 to 20;
o 0 to 20, wherein the residues R_{c} and R_{d} may be the same or different in the o groups (CR_{c}R_{c}).

59. The conjugate as claimed in claim 57, wherein Rₐ, R_{b}, R_{c}, and R_{d} are hydrogen, m=2, n=1, and o=2.

60. A conjugate comprising a protein and a polymer or a derivative thereof, wherein the polymer is a hydroxyalkyl starch (HAS) and the protein is a granulocyte colony stimulating factor (G-CSF), having a structure according to the formula 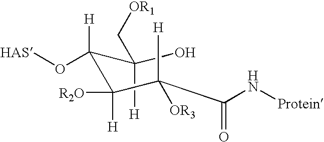
wherein R₁, R₂ and R₃ are independently hydrogen or a hydroxyalkyl group, a hydroxyaryl group, a hydroxyaralkyl group or a hydroxyalkaryl group having of from 2 to 10 carbon atoms.

61. A conjugate comprising a protein and a polymer or a derivative thereof, wherein the polymer is a hydroxyalkyl starch (HAS) and the protein is a granulocyte colony stimulating factor (G-CSF), having a structure according to the formula 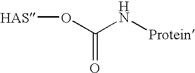
wherein R₁, R₂ and R₃ are independently hydrogen or a hydroxyalkyl group, a hydroxyaryl group, a hydroxyaralkyl group or a hydroxyalkaryl group having of from 2 to 10 carbon atoms, and wherein the linkage —O—(C═O)— was formed by a reaction of a carboxy group or a reactive carboxy group with a hydroxy group of the HAS molecule, and wherein HAS″ refers to the HAS molecule without said hydroxy group.

62. A conjugate, comprising a protein and a polymer or a derivative thereof, wherein the polymer is a hydroxyalkyl starch (HAS) and the protein is a granulocyte colony stimulating factor (G-CSF), having a structure according to the formula 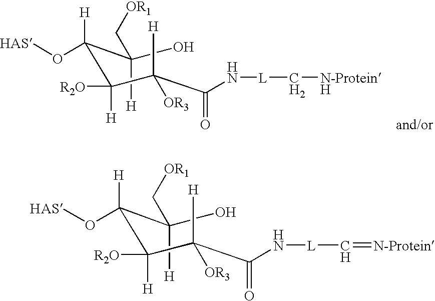
wherein R₁, R₂ and R₃ are independently hydrogen or a hydroxyalkyl group, a hydroxyaryl group, a hydroxyaralkyl group or a hydroxyalkaryl group having of from 2 to 10 carbon atoms, and wherein L is an optionally substituted, linear, branched and/or cyclic hydrocarbon residue, optionally comprising at least one heteroatom, having from 1 to 60 carbon atoms.

63. A conjugate, comprising a protein and a polymer or a derivative thereof, wherein the polymer is a hydroxyalkyl starch (HAS) and the protein is a granulocyte colony stimulating factor (G-CSF), having a structure according to the formula 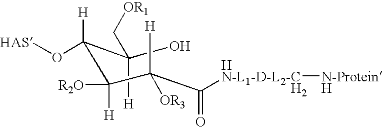
wherein R₁, R₂ and R₃ are independently hydrogen or a hydroxyalkyl group, a hydroxyaryl group, a hydroxyaralkyl group or a hydroxyalkaryl group having of from 2 to 10 carbon atoms, and wherein L₁ and L₂ are independently an optionally substituted, linear, branched and/or cyclic hydrocarbon residue, optionally comprising at least one heteroatom, comprising an alkyl, aryl, aralkyl heteroalkyl, and/or heteroaralkyl moiety, said residue having from 1 to 60 carbon atoms, and wherein D is a linkage formed by a suitable functional group F₂ linked to L1 and a suitable functional group F₃ linked to L₂.

64. The conjugate as claimed in claim 63, wherein L1 is —(CH₂)ₙ— with n=2, 3, 4, 5, 6, 7, 8, 9, or 10.

65. The conjugate as claimed in claim 63, wherein L₂ comprises an optionally suitably substituted aryl moiety.

66. The conjugate as claimed in claim 63, wherein is F₂ selected from the group consisting of
C—C-double bonds or C—C-triple bonds or aromatic C—C-bonds;
thiol groups or hydroxy groups;
alkyl sulfonic acid hydrazide, aryl sulfonic acid hydrazide;
1,2-dioles;
1,2 amino-thioalcohols;
azides;
1,2-aminoalcohols;
the amino group —NH₂ or derivatives of the amino groups comprising the structure unit —NH— such as aminoalkyl groups, aminoaryl groups, aminoaralkyl groups, or alkarlyamino groups;
the hydroxylamino group —O—NH₂, or derivatives of the hydroxylamino group comprising the structure unit —O—NH—, such as hydroxylalkylamino groups, hydroxylarylamino groups, hydroxylaralkylamino groups, or hydroxalalkarylamino groups;
alkoxyamino groups, aryloxyamino groups, aralkyloxyamino groups, or alkaryloxyamino groups, each comprising the structure unit —NH—O—;
residues having a carbonyl group, -Q-C(=G)-M, wherein G is O or S, and M is
—OH or —SH;
an alkoxy group, an aryloxy group, an aralkyloxy group, or an alkaryloxy group;
an alkylthio group, an arylthio group, an aralkylthio group, or an alkarylthio group;
an alkylcarbonyloxy group, an arylcarbonyloxy group, an aralkylcarbonyloxy group, an alkarylcarbonyloxy group;
activated esters such as esters of hydroxyl amines having imid structure such as N-hydroxysuccinimide or having a structure unit O—N where N is part of a heteroaryl compound or, with G=O and Q absent, such as aryloxy compounds with a substituted aryl residue such as pentafluorophenyl, paranitrophenyl or trichlorophenyl;
wherein Q is absent or NH or a heteroatom such as S or O;
—NH—NH₂, or —NH—NH—;
—NO₂;
nitril groups;
carbonyl groups such as the aldehyde group or the keto group;
carboxy group groups;
a —N═C═O group or a —N═C═S group;
vinyl halide groups such as vinyl iodide or vinyl bromide group or triflate;
—C═C—H;
—(C═NH₂Cl)—OAlkyl
groups —(C═O)—CH₂-Hal wherein Hal is Cl, Br, or I;
—CH═CH—SO₂—;
a disulfide group comprising the structure —S—S—;
the group 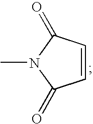
the group 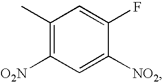
and wherein F₃ is a functional group capable of forming a chemical linkage with F₂.

67. A conjugate, comprising a protein and a polymer or a derivative thereof, wherein the polymer is a hydroxyalkyl starch (HAS) and the protein is a granulocyte colony stimulating factor (G-CSF), having a structure according to the formula 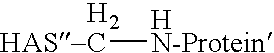
wherein the carbon atom of the moiety —CH₂—N₂— is derived from an aldehyde group which was introduced in the polymer by a ring-opening oxidation reaction, and wherein the nitrogen atom is derived from an amino group of the protein.

68. A conjugate, comprising a protein and a polymer or a derivative thereof, wherein the polymer is a hydroxyalkyl starch (HAS) and the protein is a granulocyte colony stimulating factor (G-CSF), having a structure according to the formula 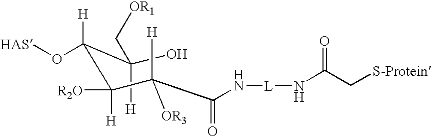
wherein R₁, R₂ and R₃ are independently hydrogen or a hydroxyalkyl group, a hydroxyaryl group, a hydroxyaralkyl group or a hydroxyalkaryl group having of from 2 to 10 carbon atoms, and wherein L is an optionally substituted, linear, branched and/or cyclic hydrocarbon residue, optionally comprising at least one heteroatom, comprising an alkyl, aryl, aralkyl heteroalkyl, and/or heteroaralkyl moiety, said residue having from 2 to 60 carbon atoms, and wherein the sulfur atom is derived from a cysteine residue or a disulfide group of the protein.

69. The conjugate as claimed in claim 68, wherein -L- is
—[(CRₐR_{b})ₘG]ₙ[CR_{c}R_{d}]ₒ—
wherein Rₐ, R_{b}, R_{c}, and R_{d} are independently hydrogen, alkyl, or aryl, wherein G is selected from the group consisting of O and S, and wherein
m 1, 2, 3 or 4, wherein the residues Rₐ and R_{b} may be the same or different in the m groups (CRₐR_{b});
n 1 to 20;
o 1 to 20, wherein the residues R_{c} and R_{d} may be the same or different in the o groups (CR_{c}R_{d});
or wherein
n 0, and
o 2 to 20, wherein the residues R_{c} and R_{d} may be the same or different in the o groups CR_{c}R_{d}.

70. A conjugate, comprising a protein and a polymer or a derivative thereof, wherein the polymer is a hydroxyalkyl starch (HAS) and the protein is a granulocyte colony stimulating factor (G-CSF), having a structure according to the formula 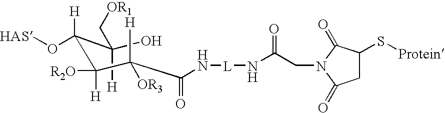
wherein R₁, R₂ and R₃ are independently hydrogen or a hydroxyalkyl group, a hydroxyaryl group, a hydroxyaralkyl group or a hydroxyalkaryl group having of from 2 to 10 carbon atoms, and wherein L is an optionally substituted, linear, branched and/or cyclic hydrocarbon residue, optionally comprising at least one heteroatom, comprising an alkyl, aryl, aralkyl heteroalkyl, and/or heteroaralkyl moiety, said residue having from 2 to 60 carbon atoms, and wherein the sulfur atom is derived from a cysteine residue or a disulfide group of the protein.

71. The conjugate as claimed in claim 70, wherein -L- is
—[(CRₐR_{b})ₘG]ₙ[CR_{c}R_{d}]ₒ—
wherein Rₐ, R_{b}, R_{c}, and R_{d} are independently hydrogen, alkyl, or aryl, wherein G is selected from the group consisting of O and S, and wherein
m 1, 2, 3 or 4, wherein the residues Rₐ and R_{b} may be the same or different in the m groups CRₐR_{b};
n 1 to 20;
o 1 to 20, wherein the residues R_{c} and R_{d} may be the same or different in the o groups CR_{c}R_{d};
or wherein
n 0, and
o 2 to 20, wherein the residues R_{c}, and R_{d} may be the same or different in the o groups CR_{c}R_{d}.

72. The conjugate as claimed in claim 54, wherein the hydroxyalkyl starch is hydroxyethyl starch.

73. The conjugate as claimed in claim 72 wherein the hydroxyethyl starch has a molecular weight of from 2 to 200 kD.

74. (canceled)

75. A pharmaceutical composition comprising in a therapeutically effective amount a conjugate as claimed in claim 54.

76. A pharmaceutical composition as claimed in claim 75, further comprising at least one pharmaceutically acceptable diluent, adjuvant, or carrier.

77. A method for treatment of a disorder characterized by a reduced hematopoietic or immune function, comprising administering to a subject in need thereof a therapeutically effective amount of the conjugate as claimed in claim 54.

78. The method as claimed in claim 77, wherein the disorder is a result of chemotherapy, radiation therapy, infectious disease, severe chronic neutropenia, or leukemia.
